SEQUENCE LISTING (25 sequences)

SEQ ID NO: 1 - DNA, 1323 bp, Mus musculus
atggctggtgtctcccctgctgtctttggctgcccagatgtcaccctgggaaggaacactgcagtccgtgaagtacaagaaaatgtcacatcagtggacagtttaacactggtctccagcaacactgactttgccttcagcctctacaggaagctggttttgaagaatccagatgaaaatgttgtcttctccccattcagcatctgcactgccttggccctcctgtccctgggagcaaagagcaacaccctgaaggaaatcctagaaggtctcaagttcaacctcacagagacccctgaaccagacatccaccagggctttaggtacttgctagaccttctaagtcagccagggaaccaggtacagatcagcacaggcagtgccctgtttattgaaaagcacctacagatcctggcagagttcaaggagaaggcaagggctctgtaccaggctgaggccttcacagcagatttccagcaacctctcaaggccacaaagctcatcaatgactatgtgagcaatcacacccaggggaagatcaaggaactcatctcaggcctgaaagagagcacgttgatggtgctggtgaactacatctactttaaaggcaaatggaagaacccctttgacccgaatgatacatttaagtccgagttctacttggatgagaagaggtctgtgattgtgtccatgatgaaaactggttacctgacgacaccctacttccgggatgaggagctgtcctgcactgtggtggagctgaagtacacaggaaatgccagtgccatgttcatcctccctgaccagggcaggatgcagcaggtggaagcaagcttacaaccagagaccctgaggaagtggaagaactctctgaagcccaggatgatacatgagctccgcctgcccaagttctccatctccaccgactatagcctggagcacatccttcctgagttgggcatcagggaagtcttctccacacacgctgacctgtctgcaatcacaggaactaaggatctgagagtctctcaggtggtccacaaggctgtgctggatgtggctgagaaaggcacagaggctgctgctgccacaggaatggcaggtgtcggatgttgtgcagtttttgactttctggaaatatttttcaacaggccattcctgatgattatctctgacacaaaagctcacattgccctctttatggcaaaagttacaaatccagagagatctacgaacttcccaaatggtgagggtgcttcttcccaacggctggaatctaagcgtttatgttttggtgatccgctatgcctgattggacagtga

SEQ ID NO: 2 - protein, 440 aa, Mus musculus
MAGVSPAVFGCPDVTLGRNTAVREVQENVTSVDSLTLVSSNTDFAFSLYRKLVLKNPDENVVFSPFSICTALALLSLGAKSNTLKEILEGLKFNLTETPEPDIHQGFRYLLDLLSQPGNQVQISTGSALFIEKHLQILAEFKEKARALYQAEAFTADFQQPLKATKLINDYVSNHTQGKIKELISGLKESTLMVLVNYIYFKGKWKNPFDPNDTFKSEFYLDEKRSVIVSMMKTGYLTTPYFRDEELSCTVVELKYTGNASAMFILPDQGRMQQVEASLQPETLRKWKNSLKPRMIHELRLPKFSISTDYSLEHILPELGIREVFSTHADLSAITGTKDLRVSQVVHKAVLDVAEKGTEAAAATGMAGVGCCAVFDFLEIFFNRPFLMIISDTKAHIALFMAKVTNPERSTNFPNGEGASSQRLESKRLCFGDPLCLIGQ

SEQ ID NO: 3 - protein, 379 aa, Homo sapiens
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFCMLMPEENFTADHPFLFFIRHNSSGSILFLGRFSSP

SEQ ID NO: 4 - protein, 415 aa, Homo sapiens
MEDLCVANTLFALNLFKHLAKASPTQNLFLSPWSISSTMAMVYMGSRGSTEDQMAKVLQFNEVGANAVTPMTPENFTSCGFMQQIQKGSYPDAILQAQAADKIHSSFRSLSSAINASTGNYLLESVNKLFGEKSASFREEYIRLCQKYYSSEPQAVDFLECAEEARKKINSWVKTQTKGKIPNLLPEGSVDGDTRMVLVNAVYFKGKWKTPFEKKLNGLYPFRVNSAQRTPVQMMYLREKLNIGYIEDLKAQILELPYAGDVSMFLLLPDEIADVSTGLELLESEITYDKLNKWTSKDKMAEDEVEVYIPQFKLEEHYELRSILRSMGMEDAFNKGRANFSGMSERNDLFLSEVFHQAMVDVNEEGTEAAAGTGGVMTGRTGHGGPQFVADHPFLFLIMHKITNCILFFGRFSSP

SEQ ID NO: 5 - protein, 390 aa, Homo sapiens
MNSLSEANTKFMFDLFQQFRKSKENNIFYSPISITSALGMVLLGAKDNTAQQIKKVLHFDQVTENTTGKAATYHVDRSGNVHHQFQKLLTEFNKSTDAYELKIANKLFGEKTYLFLQEYLDAIKKFYQTSVESVDFANAPEESRKKINSWVESQTNEKIKNLIPEGNIGSNTTLVLVNAIYFKGQWEKKFNKEDTKEEKFWPNKNTYKSIQMMRQYTSFHFASLEDVQAKVLEIPYKGKDLSMIVLLPNEIDGLQKLEEKLTAEKLMEWTSLQNMRETRVDLHLPRFKVEESYDLKDTLRTMGMVDIFNGDADLSGMTGSRGLVLSGVLHKAFVEVTEEGAEAAAATAVVGFGSSPTSTNEEFHCNHPFLFFIRQNKTNSILFYGRFSSP

SEQ ID NO: 6 - protein, 390 aa, Homo sapiens
MNSLSEANTKFMFDLFQQFRKSKENNIFYSPISITSALGMVLLGAKDNTAQQISKVLHFDQVTENTTEKAATYHVDRSGNVHHQFQKLLTEFNKSTDAYELKIANKLFGEKTYQFLQEYLDAIKKFYQTSVESTDFANAPEESRKKINSWVESQTNEKIKNLFPDGTIGNDTTLVLVNAIYFKGQWENKFKKENTKEEKFWPNKNTYKSVQMMRQYNSFNFALLEDVQAKVLEIPYKGKDLSMIVLLPNEIDGLQKLEEKLTAEKLMEWTSLQNMRETCVDLHLPRFKMEESYDLKDTLRTMGMVNIFNGDADLSGMTWSHGLSVSKVLHKAFVEVTEEGVEAAAATAVVVVELSSPSTNEEFCCNHPFLFFIRQNKTNSILFYGRFSSP

SEQ ID NO: 7 - protein, 376 aa, Homo sapiens
MDVLAEANGTFALNLLKTLGKDNSKNVFFSPMSMSCALAMVYMGAKGNTAAQMAQILSFNKSGGGGDIHQGFQSLLTEVNKTGTQYLLRVANRLFGEKSCDFLSSFRDSCQKFYQAEMEELDFISAVEKSRKHINTWVAEKTEGKIAELLSPGSVDPLTRLVLVNAVYFRGNWDGQFDKENTEERLFKVSKNEEKPVQMMFKQSTFKKTYIGEIFTQILVLPYVGKELNMIIMLPDETTDLRTVEKELTYEKFVEWTRLDMMDEEEVEVSLPRFKLEESYDMESVLRNLGMTDAFELGKADFSGMSQTDLSLSKVVHKSFVEVNEEGTEAAAATAAIMMMRCARFVPRFCADHPFLFFIQHRKTNGILFCGRFSSP

SEQ ID NO: 8 - protein, 374 aa, Homo sapiens
MDDLCEANGTFAISLFKILGEEDNSRNVFFSPMSISSALAMVFMGAKGSTAAQMSQALCLYKDGDIHRGFQSLLSEVNRTGTQYLLRTANRLFGEKTCDFLPDFKEYCQKFYQAELEELSFAEDTEECRKHINDWVAEKTEGKISEVLDAGTVDPLTKLVLVNAIYFKGKWNEQFDRKYTRGMLFKTNEEKKTVQMMFKEAKFKMGYADEVHTQVLELPYVEEELSMVILLPDDNTDLAVVEKALTYEKFKAWTNSEKLTKSKVQVFLPRLKLEESYDLEPFLRRLGMIDAFDEAKADFSGMSTEKNVPLSKVAHKCFVEVNEEGTEAAAATAVVRNSRCSRMEPRFCADHPFLFFIRRHKTNCILFCGRFSSP

SEQ ID NO: 9 - protein, 376 aa, Homo sapiens
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAECCMESGPRFCADHPFLFFIRHNRANSILFCGRFSSP

SEQ ID NO: 10 - protein, 8 aa, Artificial Sequence
  Description of Artificial Sequence  Synthetic Peptide
MAGVGCCA

SEQ ID NO: 11 - protein, 8 aa, Artificial Sequence
  Description of Artificial Sequence  Synthetic Peptide
FVVAECCM

SEQ ID NO: 12 - DNA, 22 bp, Artificial Sequence
  Description of Artificial Sequence  Synthetic Peptide
aaccagagaccctgaggaagtg

SEQ ID NO: 13 - DNA, 17 bp, Artificial Sequence
  Description of Artificial Sequence  Synthetic Peptide
aacttgggcaggcgcag

SEQ ID NO: 14 - DNA, 32 bp, Artificial Sequence
  Description of Artificial Sequence  Synthetic Peptide
aagaactctctgaagcccaggatgatacatga

SEQ ID NO: 15 - DNA, 24 bp, Artificial Sequence
  Description of Artificial Sequence  Synthetic Peptide
ccatcaaaccattccttctgtagc

SEQ ID NO: 16 - DNA, 23 bp, Artificial Sequence
  Description of Artificial Sequence  Synthetic Peptide
agcagagattacaggacattgcg

SEQ ID NO: 17 - DNA, 25 bp, Artificial Sequence
  Description of Artificial Sequence  Synthetic Peptide
caggagagcgtgcctaccccatctg

SEQ ID NO: 18 - DNA, 32 bp, Artificial Sequence
  Description of Artificial Sequence  Synthetic Peptide
agaattcgccaccatggctggtgtctcccctg

SEQ ID NO: 19 - DNA, 38 bp, Artificial Sequence
  Description of Artificial Sequence  Synthetic Peptide
tgtggatcctccctgtcaaatcaggcagcatagcggat

SEQ ID NO: 20 - DNA, 26 bp, Artificial Sequence
  Description of Artificial Sequence  Synthetic Peptide
gtgaattcatcactacttgtcatcgt

SEQ ID NO: 21 - protein, 18 aa, Artificial Sequence
  Description of Artificial Sequence  Synthetic Peptide
NPERSTNFPNGEGASSQR

SEQ ID NO: 22 - protein, 17 aa, Artificial Sequence
  Description of Artificial Sequence  Synthetic Peptide
SLQPETLRKWKNSLKPR

SEQ ID NO: 23 - protein, 9 aa, Artificial Sequence
  Description of Artificial Sequence  Synthetic Peptide
FQPQNGQFI

SEQ ID NO: 24 - protein, 9 aa, Artificial Sequence
  Description of Artificial Sequence  Synthetic Peptide
KAVYNFATM

SEQ ID NO: 25 - protein, 11 aa, Artificial Sequence
  Description of Artificial Sequence  Synthetic Peptide
SGVENPGGYCL

(19) United States
(12) Patent Application Publication
(10) Pub. No.: US 2009/0324700 A1
(43) Pub. Date: Dec. 31, 2009

(54) METHODS AND COMPOSITIONS FOR THE INHIBITION OF CATHEPSINS

(76) Inventor: PHILIP ASHTON-RICKARDT, Chicago, IL (US)

Correspondence Address: FULBRIGHT & JAWORSKI L.L.P., 600 CONGRESS AVE., SUITE 2400, AUSTIN, TX, 78701, US

(21) Appl. No.: 12/121,592

(22) Filed: May 15, 2008

Related U.S. Application Data
(63) Continuation of application No. 10/782,401, filed on Feb. 19, 2004, now abandoned.
(60) Provisional application No. 60/448,285, filed on Feb. 19, 2003.

(51) Int. Cl. A61K 9/127; A61K 39/00; A61K 39/12; A61K 39/21; C12N 5/06; A61K 38/08; A61K 38/07

(52) U.S. Cl. 424/450; 424/185.1; 424/186.1; 424/188.1; 424/199.1; 435/377; 514/18; 514/17; 514/16

(57) ABSTRACT

Methods and compositions for modulating cell death by contacting a cell with an Spi2A polypeptide or an Spi2A polypeptide equivalent are disclosed. In addition, methods of treating a subject by providing the subject a composition that includes an Spi2A polypeptide or an Spi2A polypeptide equivalent are disclosed. The Spi2A polypeptide and Spi2A polypeptide equivalent can be delivered to the subject using gene therapy techniques. The subject can be a patient with a disease associated with an abnormal rate of cell death, such as septic shock or myocardial infarction. Also disclosed are methods of preparing and storing donor granulocytes, involving contacting the donor granulocytes with an Spi2A polypeptide or an Spi2A polypeptide equivalent.

This application claims the benefit of U.S. Provisional Application No. 60/448,285, filed on Feb. 19, 2003, which is incorporated by reference in its entirety.

The government owns rights in the present invention pursuant to grant number AM45108 from the National Institutes of Health.

### BACKGROUND OF THE INVENTION

1. Field of the Invention

The present invention relates generally to the fields of molecular biology, cell biology, and pharmacology. More particularly, it concerns methods and compositions for modulating cell death using a serine protease inhibitor 2A (Spi2A) polypeptide or a Spi2A polypeptide equivalent.

2. Description of Related Art

A wide variety of factors are involved in the control of survival of a cell. Some of these factors have been shown to be initiated within specific cellular organelles. For example, the mitochondrion is involved in the caspase-mediated apoptotic pathway of cell death. Ligation of ‘death receptors,’ such as tumor necrosis factor receptor 1 (TNF-R1), causes the release of mitochondrial proteins into the cytoplasm of a cell. The release of mitochondrial proteins into the cell triggers the caspase protease cascade, which in turn results in apoptosis (Budihardjo et al, 1999).

In addition to the mitochondrion, the lysosome also plays a role in modulating cell death (Ferri and Kroemer, 2001). Cathepsins, which are cysteine proteases, are located within lysosomes. There are eleven human cathespins (B, H, L, S, C, K, O, F, V, X and W) that are now known at the sequential level (reviewed in Turk et al., 2002). TNF-R1 can trigger cell death independently of caspases by causing lysosomes to release cathepsin B into the cytoplasm. The released cathepsin B acts as a dominant executioner protease (Foghsgaard et al., 2001).

Thus, there are two pathways of apoptotic cell death. In the caspase-independent (lysosomal) pathway, cell death is mediated by lysosomal release of cathepsins. In the caspase-dependent pathway, cell death is mediated by the caspase protease cascade.

NF-κB completely blocks the TNF-α pathway leading to apoptosis through the activation of protective genes (Beg and Baltimore, 1996). This implies that NF-κB inhibits both the caspase and lysosomal pathways of cell death. Nevertheless, no single pharmacological agent has been identified which can inhibit both pathways of cell death.

Serine protease inhibitor 2A (Spi2A) was originally described in the teratocarcinoma cell line EB22 (Inglis et al., 1991). Murine Spi2A has some features of the intracellular serpins although it is most closely related to human antichymotrypsin (Hampson et al., 1997). Curiously, the original cDNA was truncated at the 5′ end as a result of an alternative splicing event. It was subsequently shown that this serpin was part of a multigene cluster of at least nine serpins on murine chromosome 12 at a locus syntenic with human chromosome 14q32.1 (Inglis and Hill, 1991). The human locus contains the genes encoding antitrypsin, antichymotrypsin, protein C inhibitor and cortisol binding globulin (CBG). After its original description, Spi2A was identified as a gene expressed in the pluripotent hemopoietic cell line FDCP-Mix A4, which was dramatically down-regulated upon differentiation. (Hampson et al., 1997). Similarly, when granulocyte macrophage-colony forming cells (GM-CFC) were isolated from murine bone marrow and induced to differentiate, down regulation of expression could be shown. When FDCP-Mix A4 cells were stably transfected with Spi2A, they showed delayed differentiation and increased clonogenic potential (Hampson et al., 1997). Northern blot studies showed Spi2A message in lymphoid tissues and expression was markedly upregulated in primary splenocyte cultures upon T cell activation.

Inhibitors of both the caspase-dependent and caspase-independent pathways of cell death can provide a novel means of inhibiting cell death since the need to target both pathways with different agents would be overcome. In addition, these agents could be applied in the treatment of diseases and conditions associated with cell death. For example, these agents can be applied to prevent cell death associated with inflammatory diseases such as sepsis (Bochud and Calandra, 2003), hepatitis and liver cirrhosis (viral and chemical induced) (Crawford, 1999). In addition these agents can be used to treat disease caused by ischemia-induced cell death, such as myocardial infarction. (Itoh et al., 1995; Kajstura et al., 1996). These agents can also be used to prevent the apoptotic cell death that commonly occurs in donor granulocytes during the process of preparation of the granulocytes for subsequent transfusion to a recipient (Brach et al., 1992).

In the absence of caspase activity, one possible way in which cathepsin B released into the cytoplasm promotes cell death is through activation of Bid, leading to mitochondrial dysfunction and the production of damaging reactive oxygen species (ROS) (Ferri and Kroemer, 2001). The loss of lysosome integrity and the release of cathepsins and other digestive enzymes is a critical event in the induction of not only apoptosis but also coagulative necrosis (Ferri and Kroemer, 2001; Wyllie et al., 1981). Therefore, agents that can inhibit both pathways of cell death can also provide a novel means of protection against cell death and dysfunction that is related to necrosis, lysosomal instability, and ROS.

Agents that inhibit both pathways of cell death can also be applied as therapeutic agents in the treatment of diseases associated with abnormal lysosomal cysteine protease activity. When secreted, lysosomal cysteine proteases can be very harmful for their environment, resulting in pathological conditions. Cysteine proteases have been observed to be involved in a number of diseases (see, generally, Turk et al., 2002) such as rheumatoid arthritis and osteoarffiritis (Nort et al., 1984; Mort et al., 1998; Baici et al, 1988; Baici et al., 1995), Alzheimer disease (Cataldo and Nixon, 1990), multiple sclerosis (Bever and Garver, 1995) and muscular dystrophy (Takeda et al., 1992, Kominami et al., 1987). In many of these diseases, lysosomal enzymes were found to be present in the extracellular/extralysosomal environment in the proforms, which are substantially more stable than the mature enzymes. There is also evidence that lysosomal cysteine proteases are also involved in neuronal apoptosis (Nixon and Cataldo, 1993).

Cysteine proteases, in particular cathepsin B, have also been shown to be associated with malignancy (Poole et al., 1980; Sloane et al., 1981; Turk et al., 2002). Other studies have shown that cathepsins B, H and L are involved in cancer progression either by direct degradation of extracellular matrix or by activation of other proteases, such as urokinase-type plasminogen activator (reviewed in Turk et al., 2002). This involvement could be accomplished by increases in secretion, mRNA and protein levels and activity.

Therefore, the identification of modulators of both the caspase-dependent and caspase-independent mechanisms could be applied in new forms of treatment in diseases and conditions that are associated with cell death due apoptosis, necrosis, lysosomal instability, ROS, and other related mechanisms. Agents that are inhibitors of both pathways of cell death can also be used to prevent apoptosis that commonly occurs in donor granulocytes following harvesting and preparation for administration to a recipient. In addition, these agents can also be used in treating patients with conditions associated with abnormal cysteine protease activity, such as cancer.

### SUMMARY OF THE INVENTION

The inventor has discovered that Spi2A inhibits both the caspase pathway and caspase-independent pathway of cell death. In particular, it has been discovered that NF-κB complexes inhibit the cathepsin B pathway of cell death, and Spi2A is a mediator of this inhibition. It has been shown that the inhibition of the cathepsin B pathway of cell death is the result of activation of TNF-R1. TNF-R1 has been shown to induce the NFκB-dependent, up-regulation of Spi2A, a potent inhibitor of cysteine cathepsins. As described for other NF-κB target genes, the expression of Spi2A antagonizes the caspase-dependent pathway of apoptosis (Baldwin, 2001). However, since lysosomal cathepsin B can induce cell death without caspase activation, Spi2A also affords protection against caspase-independent cell death (Bomer and Monney, 1999). Therefore, a novel mechanism by which NF-κB blocks the lysosomal pathway of cell death has been identified. In view of these findings, Spi2A and Spi2A equivalents can be used as novel agents to modulate cell death in a target cell and can be used in new forms of treatment of diseases and conditions associated with cell death, lysosomal instability, and abnormal cysteine protease activity.

Certain embodiments of the present invention are generally concerned with methods of modulating cell death in a cell, which is achieved by contacting the target cell with an Spi2A polypeptide or an Spi2A polypeptide equivalent. As used herein, “Spi2A” will refer to murine Spi2A, and is further discussed in the specification below. An Spi2A polypeptide pertains to a polypeptide based on the sequence of murine Spi2A. A polypeptide of any length is contemplated by the present invention, including a polypeptide based on the full amino acid sequence of Spi2A.

“Spi2A polypeptide equivalent,” discussed in detail in the specification below, includes any Spi2A polypeptide in which some, or most, of the amino acids may be substituted so long as the polypeptide retains substantially similar activity in the context of the uses set forth herein.

For example, a Spi2A polypeptide equivalent includes a polypeptide from Serpin B1, Serpin B2, Serpin B3, Serpin B4, Serpin B6, Serpin B8, or Serpin B9. In certain particular embodiments of the present invention, a Spi2A polypeptide equivalent is a Serpin B9 polypeptide.

Other examples of Spi2A polypeptide equivalents that are anticipated to have an acceptable level of equivalent biological activity of Spi2A includes polypeptides having the amino acid sequence MAGVGCCA (SEQ ID NO:10) or polypeptides having the amino acid sequence FVVAECCM (SEQ ID NO: 11). These amino acid sequences are part of Spi2A and P19, respectively. The Spi2A polypeptide equivalents may include all or part of these amino acid sequences. For example, the Spi2A polypeptide equivalent may include 8, 7, 6, 5, or 4 consecutive amino acids in forward or reverse orientation from either of these amino acid sequences. Any number of additional amino acid residues may be located at the C-terminal or N-terminal of the polypeptide.

In further embodiments, the Spi2A polypeptide or Spi2A polypeptide equivalent includes an amino acid sequence designed to facilitate incorporation of the polypeptide into the intracellular compartment of the cell. Although a person of ordinary skill in the art would understand that the Spi2A polypeptide or Spi2A polypeptide equivalent can be fused to any amino acid sequence known to facilitate internalization into the intracellular compartment, a specific embodiment involves use of a polypeptide encoding an amino acid TAT sequence from HIV. In another embodiment, the Spi2A polypeptide or Spi2A polypeptide equivalent is fused to a polypeptide encoding an Antp amino acid sequence. Still another embodiment involves fusion of an Spi2A polypeptide or Spi2A polypeptide equivalent to a polypeptide encoding a VP22 amino acid sequence from HSV.

The present invention contemplates embodiments that require use of Spi2A polypeptides and Spi2A equivalent polypeptides to modulate cell death wherein the cell death is related to any known mechanism of cell death.

In certain embodiments of the invention, the method for modulating cell death is further defined as a method for modulating apoptosis. In some embodiments, the method for modulating apoptosis is further defined as a method for modulating cell death of a T lymphocyte. Modulation of death of T lymphocytes can be applied in embodiments of the invention that are directed to methods of facilitating the differentiation of a lymphocyte into a memory T lymphocyte.

In some embodiments of the present invention, the Spi2A polypeptide or Spi2A polypeptide equivalent is comprised in a vaccine. The vaccine, for example, may be directed against a target cell in a subject, such as a tumor cell or a cell that is infected by a pathogen. For example, the tumor cell may be a cell from a breast cancer, lung cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, or leukemia. The virus can be any virus known to those of ordinary skill in the art. For example, in some embodiments the virus is HIV, HSV, or ADV. The vaccine may include additional agents that are useful in the treatment or prevention of tumors or infections by pathogens.

The apoptosis may be apoptosis that occurs as a result of increased lysosomal permeability with the cell. Increase in lysosomal permeability can result in release of lysosomal proteases. Thus, embodiments of the present invention pertain to methods of modulating cell death that is further defined as cell death due to release of at least one lysosomal protease in the cell. Although any lysosomal protease is contemplated by the present invention, in preferred embodiments the lysosomal protease is a cysteine protease. For example, the cysteine protease can be cathepsin B, cathepsin H, cathepsin L, cathepsin S, cathepsin K, cathepsin O, cathepsin F, cathepsin V, cathepsin X, or cathepsin W. The present invention also pertains to methods of modulating cell death due to autophagic cell death, TNF-α mediated cell death, cell death due to reactive oxygen species (ROS), and cell death due to necrosis.

Although one of ordinary skill in the art would understand that any cell is contemplated by the present invention, in preferred embodiments the cell is located in a subject. More specifically, the subject can be a human. The human may or may not be a patient with an underlying disease. Although any disease is contemplated by the present invention, in certain specific embodiments the disease is a disease associated with an abnormal rate of cell death. For example, the patient can have vascular disease. The vascular disease may be occlusive vascular disease or cardiovascular disease. The cardiovascular disease can be a myocardial infarction. More specifically, the myocardial infarction can be an acute myocardial infarction.

The patient can also have an infection. In a particular embodiment, the infection results in septic shock. The infectious agents may be gram negative or gram positive bacteria or a fungus. The infectious agent causing sepsis may also be a biological weapon such as Bacillus anthracis (leading to cutaneous, inhalation or intestinal anthrax) or Yersinia pestis (leading to bubonic, septicemic or pneumonic plague).

The disease can also be a disease associated with cell death due to necrosis, reactive oxygen species, or lysosomal instability. These include fulminating hepatic failure caused by hepatitis A, B, C, D, E or G virus, anti-tuberculosis drugs such as rifamycin or isoniazid, anti-depressant monoamine oxidase inhibitor drugs, industrial chemicals such as carbon tetrachloride, or alcohol. The disease may be an inflammatory disease such as hepatitis or liver cirrhosis caused by hepatitis A, B, C, D, E or G virus, anti-tuberculosis drugs such as rifamycin or isoniazid, anti-depressant monoamine oxidase inhibitor drugs, industrial chemicals such as carbon tetrachloride, or alcohol. The inflammatory disease may also be rheumatoid arthritis, or osteoarthritis. The disease can also be emphysema or osteoporosis.

In another example, the disease or condition may be one that is associated with abnormal cysteine protease activity. For example, the disease can be a bone disease, neurodegenerative disease, Alzheimer disease, viral disease such as HIV, multiple sclerosis, muscular dystrophy, or arthritis including rheumatoid arthritis and osteoarthritis. Because immune disorders have been associated with abnormal cysteine protease activity, the patient can also have an immune disorder. The immune disorder can be an autoimmune disorder or a disorder associated with abnormal antigen presentation. As discussed above, abnormal cysteine protease activity has been associated with cancer.

Therefore, in a certain embodiment the subject is a patient with cancer. The patient with cancer can be a cancer patient undergoing secondary anti-hyperplastic therapy. Examples of such secondary anti-hyperplastic therapy include chemotherapy, radiotherapy, immunotherapy, phototherapy, cryotherapy, toxin therapy, hormonal therapy or surgery.

In still further embodiments of the present invention, the Spi2A polypeptide or said Spi2A polypeptide equivalent is included in an expression cassette that further includes a promoter, active in the cell, operably linked to a polynucleotide encoding an Spi2A polypeptide or an Spi2A polypeptide equivalent. In a particular embodiment, the expression cassette includes a promoter, active in the cell, operably linked to a polynucleotide encoding an Spi2A polypeptide. In another particular embodiment, the expression cassette includes a promoter, active in the cell, operably linked to a polynucleotide encoding an Spi2A polypeptide equivalent. The polynucleotide encoding the Spi2A polypeptide or the Spi2A polypeptide equivalent may be comprised in a vaccine.

Although any Spi2A polypeptide equivalent is contemplated, in certain embodiments the Spi2A polypeptide equivalent one of the previously discussed human equivalents. The expression cassette can be carried in a viral vector. Although one of skill in the art would understand that any viral vector is contemplated by the invention, examples of a viral vector include an adenoviral vector, a retroviral vector, an adeno-associated viral vector, a vaccinia viral vector, or a pox viral vector. The expression cassette can also be carried in a nonviral vector, such as a liposome. Although use of any promoter capable of expression in the cell is contemplated by the present invention, the promoter can be a constitutive promoter, an inducible promoter or a tissue-specific promoter. In certain embodiments, the expression cassette further includes an origin of replication, a polyadenylation signal, or a selectable marker gene.

In still further embodiments of the invention, the Spi2A polypeptide or Spi2A polypeptide equivalent is obtained from media of cultured cells and applied to the surface of the cell. The cultured cells may or may not include an expression cassette. The expression cassette can include any of the characteristics that have been previously described.

Other embodiments of the invention pertain to methods of treating a subject that includes (1) providing a composition that includes an Spi2A polypeptide or an Spi2A polypeptide equivalent a pharmaceutical preparation suitable for delivery to said subject; and (2) administering the composition to the subject. In particular embodiments, the composition includes an Spi2A polypeptide. In other particular embodiments, the composition includes an Spi2A polypeptide equivalent, such as any of the previously described human Spi2A equivalents.

The method of treatment can be further defined as a method of modulating cell death in a subject. The method of modulating cell death can be a method of modulating cell death by any of the mechanisms of cell death previously described in this specification.

In still further embodiments, the method of treatment is defined as method of treating a disease or condition in a subject. A preferred subject is a human. The human can be a patient with any disease. In specific embodiments, the disease or condition is associated with cell death or abnormal cysteine protease activity. Examples of these diseases have been previously described. In a specific embodiment, the disease is septic shock. In another specific embodiment, the disease is myocardial infarction. The myocardial infarction can be an acute myocardial infarction.

In some embodiments, the method of treatment is further defined as a method of facilitating the differentiation of memory T lymphocytes wherein the memory T lymphocytes are directed against diseased cells in the subject. In some embodiments, the Spi2A polypeptide or Spi2A polypeptide equivalent is comprised in a vaccine. The diseased cell may be a tumor cell or a cell that is infected by a pathogen. For example, the tumor cell may be a cell from a breast cancer, lung cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, or leukemia. The pathogen may be a virus, such as HIV, HSV, or ADV. Vaccines are discussed in greater detail in the specification below.

In certain embodiments the composition is delivered systemically. Other examples of methods of delivery include intravascular delivery, and local delivery to a lesion such as a tumor.

Further embodiments pertain to methods of treating a subject involving administering an Spi2A polypeptide or an Spi2A polypeptide that further includes an amino acid sequence that preferentially targets the protein/polypeptide for intracellular entry. The sequence that targets the Spi2A polypeptide or the Spi2A equivalent polypeptide for intracellular entry can include any of the amino acids previously described or any such sequence known to one of skill in the art.

The composition can include an expression cassette similar to that which has been noted above. One of skill in the art would understand that a variety of experimental techniques are available to practice the claimed invention using expression cassettes, some of which are discussed in greater detail below.

Further embodiments of the invention include methods of preparing donor granulocytes for delivery to a subject in need of a granulocyte donation, including: (1) obtaining donor granulocytes from a suitable donor; (2) isolating the donor granulocytes; (3) contacting the donor granulocytes with a composition comprising an Spi2A polypeptide or an Spi2A polypeptide equivalent and a pharmaceutical preparation suitable for delivery to the donor granulocytes; and (4) administering the donor granulocytes to a subject in need of the donor granulocytes. In a particular embodiment, the composition includes an Spi2A polypeptide. In other particular embodiment, the composition includes an Spi2A equivalent polypeptide, such as the human Spi2A polypeptide equivalents previously described.

The method of preparing donor granulocytes can be further defined as a method of preparation that results in reduction of apoptosis of the donor granulocytes. The method of preparation can also result in reduction of granulocyte necrosis, reduction of lysosomal instability and reduction of cell death due to ROS. The recipient of the donor granulocytes can be any subject. However, in certain embodiments the subject is a subject with a disorder involving granulocytes. For instance, the subject can be a subject with neutropenia. The neutropenia may be neutropenia that is the result of chemotherapy, radiation therapy, myelosuppressive drug treatment leukemia, aplastic anemia, or idiopathic neutropenia. The neutropenia may or may not be associated with sepsis or septic shock. In other embodiments, the subject is a subject with a qualitative abnormality of neutrophils. For instance, the qualitative abnormality of neutrophils can be chronic granulomatous disease.

In certain embodiments of the invention, the donor granulocytes are collected from donors who may have been treated with G-CSF to boost granulocytes numbers. In certain other embodiments, the granulocytes are purified by leukapheresis.

In certain embodiments of the invention, the composition that is contacted with the donor granulocytes includes an expression cassette comprising a promoter, active in cells of the subject, operably linked to a polynucleotide encoding a Spi2A polypeptide or an Spi2A polypeptide equivalent. As previously noted, experimental techniques using expression cassettes are well-known to those of skill in the art. Some of these experimental techniques are discussed below. In a particular embodiment, the composition includes an expression cassette comprising a promoter, active in cells of the subject, operably linked to a polynucleotide encoding an Spi2A polypeptide. In other embodiments, the composition includes an expression cassette comprising a promoter, active in cells of the subject, operably linked to a polynucleotide encoding an Spi2A polypeptide equivalent such as any of the previously described human Spi2A polypeptide equivalents. In further embodiments, the Spi2A polypeptide or the Spi2A polypeptide equivalent includes an amino acid sequence such as one of the previously described amino acid sequences that are known to facilitate intracellular delivery of the protein or polypeptide sequence.

Certain other embodiments of the invention provide for methods of preparing donor granulocytes for storage, including (a) obtaining donor granulocytes from a suitable donor; (b) isolating the donor granulocytes; (c) contacting the donor granulocytes with a composition comprising an Spi2A polypeptide or an Spi2A polypeptide equivalent and a pharmaceutical preparation suitable for delivery to the donor granulocytes; and storing the donor granulocytes. In other embodiments, the method of preparating the granulocytes for storage further involves treatment of the donor with C-GSF prior to obtaining granulocytes from the donor. In still other embodiments, the method of preparating the granulocytes for storage further involves purifying the granulocytes by leukapheresis following isolation of the granulocytes.

The composition can include an Spi2A polypeptide or an Spi2A polypeptide equivalent.

For example, the Spi2A polypeptide equivalent can be a polypeptide from Serpin B1, Serpin B2, Serpin B3, Serpin B4, Serpin B6, Serpin B8, or Serpin B9. In particular embodiments, the method of preparing the donor granulocytes for storage results in reduction of apoptosis of the donor granulocytes. In still other embodiments, the Spi2A polypeptide or Spi2A polypeptide equivalent can include a polypeptide encoding an amino acid TAT sequence from HIV, a polypeptide encoding an Antp amino acid sequence, or a polypeptide encoding a VP22 amino acid sequence from HSV.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C: NF-κB antagonizes the lysosomal pathway of cell death. FIG. 1A: Percentage survival of RelA^{-/-} MEFs treatment with TNF-α (0.5 ng/ml) and CHX (0.1 μg/ml) in the presence (TNF+CA-074 Me) or absence (TNF) of CA-074 Me (30 μM). The recovery of cells was compared with those incubated with CHX alone (100% recovery) to determine the percentage of recovery. FIG. 1B: Percentage survival of RelA^{-/-} MEFs transduced by retrovirus encoding GFP alone or Rel A. The recovery of cells after 16 h was compared with those incubated with CHX alone (0.1 μg/ml) to determine the percentage of recovery (100% recovery). FIG. 1C: Cathepsin B activity in crude cytoplasmic extracts from RelA^{-/-} MEFs transduced by retrovirus encoding GFP alone or RelA after treatment with TNF-α (0.2 ng/ml) and CHX (0.1 μg/ml). This experiment is representative of two independent experiments.

FIGS. 2A-C. Induction of Spi2A by NF-κB protects from TNF-α-mediated death. FIG. 2A: Northern blots of mRNA from MEFs treated with TNF-α (0.2 ngml⁻¹) and CHX (0.1 μg/ml). FIG. 2B: Percentage survival of RelA^{-/-} MEFs transduced by retrovirus encoding GFP alone or Spi2A. The recovery of cells after 16 h was compared with those incubated with CHX alone (100% recovery) to determine the percentage of recovery. FIG. 2C: Western blot detection of Spi2A from GFP and Spi2A clones of RelA^{-/-} MEF cells and correlation with survival after treatment with TNF-α (1 ng/ml) and CHX (0.1 μg/ml).

FIGS. 3A-B. Spi2A is required for the protection of wild-type MEFs from TNF-α-induced death. FIG. 3A: Quantitation of endogenous Spi2A mRNA levels by real-time PCR in cloned RelA^{+/+} MEFs transduced by retrovirus encoding GFP alone or anti-sense Spi2A (Spi2A-A) 4 h after treatment with TNF-α (10 ng/ml) and CHX (10 μg/ml). FIG. 3B: Percentage survival of GFP clones and Spi2A-A clones of RelA^{+/+} MEFs 16 h after treatment with TNF-α and CHX (10 μg/ml).

FIG. 4. Percentage survival of GFP and SpiA-A clones of RelA^{+/+} MEFS 24 h after treatment with TNF-α (100 ng/ml).

FIGS. 5A-D. Spi2A inhibits apoptosis induced by TNF-α FIG. 5A: Western blots showing the proteolytic activation of effector molecules from RelA^{-/-} MEFs-GFP (clone 11) or Spi2A (clone 4)—after treatment with TNF-α (0.2 ng/ml) and CHX (0.1 μg/ml). Filled arrows indicate inactive pro-form and open arrows indicate active form of each protein. RelA^{-/-} MEFs-GFP (clone 11) or Spi2A (clone 4)—were treated with TNF-α and CHX as above and the following measured: FIG. 5B: caspase activity; FIG. 5C: mitochondrial depolarization; and FIG. 5D: ROS.

FIGS. 6A-B. The protease specificity of Spi2A. FIG. 6A: SDS-PAGE showing Spi2A (lane P-53 kD) purified from lysates (lane L) of RelA^{-/-} MEFs transduced with retrovirus encoding Spi2A-3×FLAG. FIG. 6B: Inhibition of proteases by Spi2A. The activity of protease after pre-incubation with Spi2A was compared with activity from protease incubated alone (0% inhibition) and was ±SEM from 3-4 independent experiments with assays performed in duplicate.

FIGS. 7A-C. Spi2A antagonizes the lysosomal pathway of cell death. FIG. 7A: Cathepsin B activity in crude cytoplasmic extracts from cloned RelA^{-/-} MEFs transduced by retrovirus encoding GFP alone or Spi2A after treatment with TNF-α and CHX as described before. FIG. 7B: Percentage survival of GFP and Spi2A clones of Rel A^{-/-} MEFs 2 h after treatment with sphingosine. FIG. 7C: Cathepsin B activity in crude cytoplasmic extracts from cloned RelA^{+/+} MEFs transduced by retrovirus encoding GFP alone or anti-sense Spi2A (Spi2A-A) after treatment with TNF-α. (10 ng/ml) and CHX (10 μg/ml).

FIG. 8. Spi2A offers partial protection of lysosomal de-acidification in RelA^{+/+} MEFs. RelA^{-/-} MEFs were transduced by retrovirus encoding GFP alone or Spi2A as indicated by the percentage AO-low cells after treatment with TNF-α (0.2 ng/ml) and CHX (0.1 μg/ml). The percentage of intact lysosomes was determined by staining with AO as has been described previously (Zhao et al., 2000). Briefly, cells were incubated with AO (5 μg/ml), washed and collected for flow cytometric assessment of uptake into intact lysosomes as indicated by red fluorescence (FL3 channel).

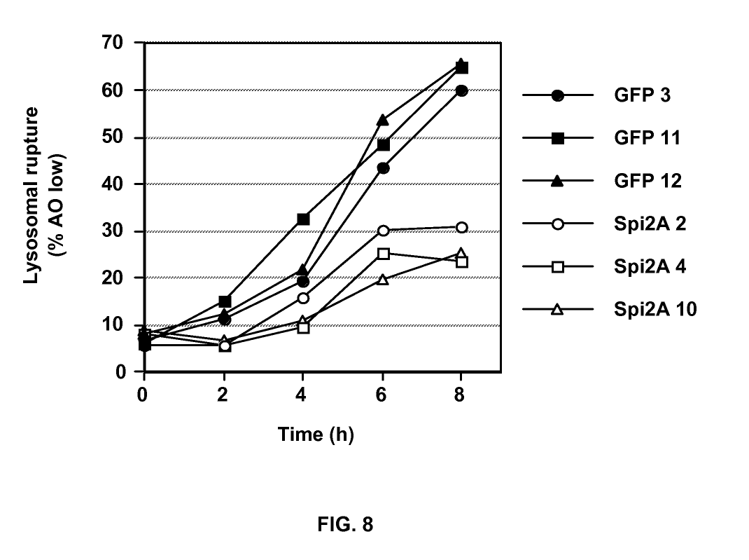

FIGS. 9A-B. Spi2A protects NIH3T3 cells from caspase-independent death induced by TNF-α. FIG. 9A: Percentage survival of NIH3T3 cells after treatment with Z-VAD.fmk (50 μM) alone or TNF-α (10 ng/ml) alone or both. The recovery of cells after 16 h was compared with those incubated alone (100% recovery) to determine the percentage of recovery. FIG. 9B: Percentage survival of clones of NIH3T3 cells transduced with retrovirus encoding GFP alone (GFP) or Spi2A (Spi2A cells) after treatment with TNF-α+Z-VAD.fmk (50 μM).

FIGS. 10A-B. Spi2A is a physiological inhibitor of caspase-independent death. FIG. 10A: Quantitation of endogenous Spi2A mRNA levels by real-time PCR in cloned NIH3T3 cells transduced by retrovirus encoding GFP alone (GFP clones) or anti-sense Spi2A (Spi2A-A clones) 4 h after treatment with either Z-VAD.fmk (50 μM) or Z-VAD.fmk+TNF-α (10 ng/ml). FIG. 10B: Percentage survival of cloned GFP or Spi2A-A NIH3T3 cells after treatment with TNF-α+Z-VAD.fmk (50 μM).

FIGS. 11A-B. Spi2A inhibits mitochondrial PCD in the absence of caspase activity. Cloned GFP or Spi2A-A NIH3T3 cells were treated with TNF-α (10 μg/ml)+Z-VAD.fmk (50 μL then FIG. 11A: mitochondrial depolarization, and FIG. 11B: ROS production measured over time.

FIGS. 12A-B: Spi2A inhibits the lysosomal pathway of death in the absence of caspase activity. FIG. 12A: Cathepsin B activity in crude cytoplasmic extracts from cloned GFP or Spi2A-A NIH3T3 cells before (time=00 h) or after (time=8 h) treatment with TNF-α+Z-VAD.fmk (50 μM). FIG. 12B: Spi2A protects NIH3T3 cells from death due to reactive oxygen species. NIH3T3 fibroblasts from independent clones harboring control retrovirus (GFP clones #, 18, 12 and 2) or one expressing Spi2A (Spi2A clones# 6, 4 and 2) were incubated with Naphazarin—a known initiator of Reactive Oxygen Species (ROS). After 16 hours, the percentage of live cells was determined by flow cytometry as described in Liu et al. (2003). A significantly increased survival of cells from all three clones expressing Spi2A compared to GFP controls was observed.

FIGS. 13A-B. Gene expression in CD8 cell populations. FIG. 13A: Splenocytes were isolated from uninfected C57BL/6 mice (naïve) or either 8d (effector) or ≧80 d (memory) after infection with LCMV. NaYve (CD44^{low}CD8⁺) cells were directly isolated from splenocytes and purified by FACS using antibodies. Splenocytes isolated from infected mice were first enriched for T cells then stained with H-2 D^{b} tetramers loaded with all three immunodominant LCMV peptides and anti-CD8a mAb. The percentages of each population prior to and after FACS are indicated. FIG. 13B: Real-time PCR analysis was performed on cDNA generated from purified CD8 cells. Data are reported as a ratio of the amount of expression of the candidate gene compared to that of the housekeeping cyclophilin A control gene. For a given candidate gene, the black and white histograms represent RNA ratios from cells purified from two independent experiments. Gene name abbreviations are: Granzyme B (Grn B), Fas Ligand (FasL), C—C chemokine receptor 5 (CCR5), Lipopolysaccharide-induced Tumor necrosis factor activation factor (LITAF), Serineprotease inhibitor 2A (Spi2A), C—C chemokine ligand 9 (CCL9), Presenilin 2 (PS2), Major histocompatibility complex I-Aab (MHC II). Brackets connecting paired histograms indicate statistically significant differences in gene expression between CD8 populations (***p<0.001, **p<0.01, *p<0.05).

FIGS. 14A-B. Modulation of Spi2A expression in bone-marrow chimeras. CD8-deficient C57BL/6 mice were re-constituted with bone-marrow progenitors transduced with recombinant MIGR1 (GFP, Spi2A or Spi2A-A mice). Chimeras with a high level of transduced leucocytes (>40% PBLs GFP⁺) were infected with LCMV. FIG. 14A: FACS purification of GFP⁺ CD8⁺ spleen cells 8d after the infection of GFP-mice with LCMV. FIG. 14B: The relative level of Spi2A mRNA from FACS purified CD8 cells. The bar indicates the mean level of Spi2A mRNA from multiple individual mice. Real-time PCR was used to determine the relative level of sense Spi2A mRNA. The mean levels of Spi2A mRNA in CD8 cells from Spi2A and Spi2A-A mice were significantly higher and lower compared to CD8 cells from GFP mice respectively.

FIG. 15. Kinetics of anti-LCMV CD8 cell expansion and contraction in bone-marrow chimeras. Wild-type C57BL/6 bone-marrow was transduced with control GFP retrovirus and adoptively transferred into lethally irradiated (1200 rads) C57BL/6 CD8-deficient mice (1.5-2.0×10⁶ cells/mouse). After either 8 or 16 weeks, the level of reconstitution was determined by measuring the percentage of CD8 cells in PBLs. After 8 weeks, chimeras were reconstituted to about 50% of the level of age-matched wild-type C57BL/6 control mice and after 16 weeks chimeras were fully reconstituted (100% of control level). Chimeras (8 week or 16 week) and control wild-type C57BL/6 mice were infected with LCMV Armstrong (2×10⁵ pfu/mouse) and the level of anti-LCMV CD8 cells was determined in PBLs staining with a H-2 D^{b}-tetramer cocktail and then FACS. The kinetics of anti-LCMV CD8 cell expansion and contraction in wild-type C57BL/6 mice was the same as has been observed by others (Murali-Krishna et al., 1998; Murali-Krishna et al., 1999). That is to say, a peak level of about 18% LCMV-specific CD8 cells after 8 days, a contraction phase that lasted until about day 30, and a residual level of about 2% LCMV-specific CD8 cells, which was about 11% of the peak level, were observed. Fully reconstituted week 16 bone-marrow chimeras exhibited the same kinetics of anti-LCMV CD8 cell expansion and contraction as wild-type C57BL/6 mice. Partially reconstituted week 8 bone-marrow chimeras exhibited altered kinetics of anti-LCMV CD8 cell expansion and contraction, with a delayed (day 14) but higher peak level and a prolonged contraction phase. The residual level of 2% LCMV-specific CD8 cells was about 4% of the peak level. Importantly, all week 8 chimeras (GFP, Spi2A, Spi2A-A) that were analyzed in a given infection were generated from the same number of bone-marrow precursors at the same time and so are matched for the degree of CD8 cell reconstitution and therefore show similar kinetics of anti-LCMV CD8 cell expansion and contraction. Week 8 rather than week 16 chimeras were chosen for infection because it allowed for performance of more experiments in a shorter period of time.

FIGS. 16A-D. Spi2A determines the level of antigen-specific CD8 cells after infection with LCMV. Bone-marrow chimeras (GFP, Spi2A or Spi2A-A) were infected with LCMV and the level of virus specific CD8 cells was determined by staining PBLs with tetramers and anti-CD8a mAbs then FACS. FIG. 16A: GFP-positive cells (transduced with retrovirus) were detected by FACS and the percentage of anti-LCMV CD8 (tetramer⁺ CD8⁺) cells of the GFP-positive population was determined by the mean±SEM from 5-6 mice at each time point. FIG. 16B: Residual level of anti-LCMV CD8 cells was determined as the percentage of the level after 98 days of the maximum level after 14 days from FIG. 16A. The residual level of anti-LCMV CD8 cells was significantly higher in Spi2A and lower in Spi2A-A mice compared to GFP controls. All of these data are representative of one of two independent experiments. FIG. 16C: Percentage of tetramer⁺ CD8⁺ of total CD8⁺ cells within the GFP-positive population of PBLs from the experiment described in part A. FIG. 16D: Percentage of anti-LCMV CD8 cells of the GFP-negative (not transduced with retrovirus) population from the experiment described in FIG. 16A.

FIG. 17. Spi2A affects the level of LCMV-specific CD8 cells. Bone-marrow chimeras (GFP, Spi2A or Spi2A-A) were infected with LCMV and the level of virus specific CD8 cells was determined by staining PBLs with tetramers and anti-CD8a mAbs then FACS. GFP-positive cells (transduced with retrovirus) were detected by FACS and the percentage of anti-LCMV CD8 (tetramer⁺ CD8⁺) cells of the GFP-positive population was determined by the mean±SEM from 5-6 mice at each time point. A decrease in the level of anti-LCMV CD8 cells at the peak response on day 14 and thereafter was observed in Spi2A-A mice compared to GFP controls. However, Spi2A mice exhibited an elevated level of anti-LCMV CD8 cells during the contraction and memory phases.

FIG. 18. Spi2A affects the contraction phase of anti-LCMV CD8 cells. The level of anti-LCMV CD8 cells present 56 days after infection was expressed as a percentage of the maximum level on day 14 to determine the residual level (data from FIG. 17). Compared to GFP controls, a significantly (p<0.001) higher residual level in Spi2A mice and a significantly lower level in Spi2A-A mice (p<0.01) were observed. These findings are similar to those in FIG. 16D, but from another independent experiment.

FIG. 19. The effect of Spi2A on the levels of memory and recall CD8 cells after infection with LCMV. Bone-marrow chimeras (GFP, Spi2A and Spi2A-A mice) were infected with LCMV and ex vivo IFN-γ production assays were performed to detect memory CD8 cells 101 d after primary infection with LCMV (memory). In another experiment, 60d after primary infection with LCMV, mice were re-infected and after 5 d the level of secondary effectors determined (recall). The percentages of IFN-γ⁺ CD8⁺ cells in the GFP-positive (+) and negative (−) populations are indicated. IFN-γ⁺ CD8⁺ cells could not be detected in spleen cells from un-infected C57BL/6 mice.

FIGS. 20A-F. Spi2A determines the level of anti-LCMV memory and recall effector CD8 cells. Bone-marrow chimeras (GFP, Spi2A and Spi2A-A mice) were infected with LCMV as described in FIG. 19. FIG. 20A: The mean percentage of IFN-γ⁺ CD8⁺ memory cells (bar) was significantly higher in Spi2A mice (n=6) and lower in Spi2A-A mice (n=5) than in GFP control mice (n=6). FIG. 20B: Percentage of IFN-γ⁺ CD8⁺ of CD8⁺ cells within the GFP-positive splenocytes from the experiment described in part A. FIG. 20C: The percentage of IFN-γ⁺ CD8⁺ memory cells of GFP-negative splenocytes from FIG. 20A showed no significant differences among the three chimera groups. FIG. 20D: The mean percentage of IFN-γ⁺ CD8⁺ recall effector cells was significantly higher in Spi2A mice (n=4) and lower in Spi2A-A mice (n=4) than in GFP control mice (n=4). These data are representative of one of two independent experiments. FIG. 20E: Percentage of IFN-γ⁺ CD8⁺ of CD8⁺ cells within the GFP-positive splenocytes from the experiment described in FIG. 20C. FIG. 20F: The percentage of IFN-γ⁺ CD8⁺ recall cells of GFP-negative splenocytes from FIG. 20D shows no significant differences among the three chimera groups.

### DESCRIPTION OF ILLUSTRATIVE EMBODIMENTS

The present invention seeks to exploit the inventors's discovery by providing for methods and compositions for simultaneously inhibiting both the caspase pathway and caspase-independent pathway of cell death using Spi2A polypeptides and mimetics of Spi2A polypeptides. These methods and compositions can be used in a wide variety of therapeutic contexts. For example, inhibition of cell death using Spi2A polypeptides or Spi2A polypeptide equivalents can be used in the treatment of diseases associated with cell death, such as septic shock and myocardial infarction. In another example, Spi2A polypeptides or Spi2A polypeptide equivalents can be used to inhibit apoptosis in donor granulocytes that are in preparation for delivery to a recipient.

### A. SPI2A

1. Spi2A Polypeptides and Fusion Proteins

The present invention pertains to use of Spi2A polypeptides or Spi2A polypeptide equivalents in various contexts. For example, various embodiments of the present invention pertain to methods for modulating cell death comprising contacting a cell with an Spi2A polypeptide or a Spi2A polypeptide equivalent. Other embodiments pertain to methods of treating a subject which include administering to the subject a composition that further includes an Spi2A polypeptide or a Spi2A polypeptide equivalent. Further embodiments of the present invention relate to methods of preparing donor granulocytes for delivery to a subject, involving contacting the donor granulocytes with a composition that includes an Spi2A polypeptide or a Spi2A polypeptide equivalent.

Throughout this application, the term “Spi2A polypeptide” is intended to refer to a murine Spi2A polypeptide. The full-length amino acid sequence of murine Spi2A is provided herein, and is designated SEQ ID NO:2.

The Spi2A polypeptide is a consecutive amino acid segment of SEQ ID NO:2 that is of any length, including the full length sequence of SEQ ID NO:2. For example, the Spi2A polypeptide can be a polypeptide that includes 4, 5, 10, 15, 20, 25, 30, 50, 100, 200, 300, 400, 500, 1000 or any number of consecutive amino acids of SEQ ID NO:2. One of ordinary skill in the art would understand how to generate a Spi2A polypeptide in view of the disclosure of SEQ ID NO:2 using any of a number of experimental methods well-known to those of skill in the art.

It is well understood by the skilled artisan that, inherent in the definition of a “Spi2A polypeptide equivalent,” is the concept that there is a limit to the number of changes that may be made within a defined portion of the molecule and still result in a molecule with an acceptable level of equivalent biological activity, e.g., ability of Spi2A to modulate cell death. “Spi2A polypeptide equivalent” is thus defined herein as any Spi2A polypeptide in which some, or most, of the amino acids may be substituted so long as the polypeptide retains substantially similar activity in the context of the uses set forth herein.

An amino acid sequence of any length is contemplated within the definition of Spi2A polypeptide equivalent, so long as the polypeptide retains an acceptable level of equivalent biological activity. For example, a Spi2A polypeptide equivalent that is anticipated to have an acceptable level of equivalent biological activity of Spi2A includes polypeptides having the amino acid sequence MAGVGCCA (SEQ ID NO:10) or polypeptides having the amino acid sequence FVVAECCM (SEQ ID NO:11). These amino acid sequences are part of Spi2A and PI9, respectively. The Spi2A polypeptide equivalents may include all or part of these amino acid sequences. For example, the Spi2A polypeptide equivalent may include 8, 7, 6, 5, or 4 consecutive amino acids from either of these amino acid sequences. The orientation of these consecutive amino acids in the Spi2A polypeptide equivalent may be forward or reverse. Further, Spi2A polypeptide equivalents includes polypeptides containing these amino acid sequences that have additional amino acids at either the C-terminal or N-terminal end. For example, the Spi2A polypeptide equivalent may include a total of greater than 1000, 500-1000, 400-499, 300-399, 200-299, 100-199, 80-99, 60-79, 50-59, 40-49, 30-39, 20-29, 10-19, 9, 8, 7, 6, 5, or 4 amino acid residues, as long as there remains an acceptable level of equivalent biological activity of Spi2A.

Of course, a plurality of distinct proteins/polypeptides/peptides with different substitutions may easily be made and used in accordance with the invention. Additionally, in the context of the invention, an Spi2A polypeptide equivalent can be a Spi2A homologue polypeptide from any species or organism, including, but not limited to, a human polypeptide. One of ordinary skill in the art will understand that many Spi2A polypeptide equivalents would likely exist and can be identified using commonly available techniques. Particular examples of Spi2A equivalents in human include serpin B1 (M/NEI; GenBank accession number AAC31394; herein SEQ ID NO:3), serpin B2 (PAI-2; GenBank accession number NP 002566; herein SEQ ID NO:4), serpin B3 (SCCA-1; GenBank accession number AAA86317; herein SEQ ID NO:5), serpin B4 (SCAA 2; GenBank accession number XP 209106; herein SEQ ID NO:6), serpin B6 (PI6; GenBank accession number NP 004559; herein SEQ ID NO:7), serpin B8 (PI8; GenBank accession number NP 002631; herein SEQ ID NO:8), and serpin B9 (PI9; GenBank accession number AAH02538; herein SEQ ID NO:9). Of course, any Spi2A homologue polypeptide may be substituted in some, even most, amino acids and still be an “Spi2A polypeptide equivalent,” so long as the polypeptide retains substantially similar activity in the context of the uses set forth herein.

These human ammo acid sequences have an amino acid identity of about 40% with murine Spi2A (SEQ ID NO:2), and a chemical identity (presence of identical or chemically similar amino acids) of about 60-70%, indicating that they are biologically equivalent polypeptides to Spi2A. Therefore, these human polypeptides are Spi2A equivalent polypeptides because only certain amino acids are substituted when compared to Spi2A.

The present invention may utilize Spi2A polypeptides or Spi2A polypeptide equivalents purified from a natural source or from recombinantly-produced material. Those of ordinary skill in the art would know how to produce these polypeptides from recombinantly-produced material. This material may use the 20 common amino acids in naturally synthesized proteins, or one or more modified or unusual amino acids. Generally, “purified” will refer to an Spi2A composition that has been subjected to fractionation to remove various other proteins, polypeptides, or peptides, and which composition substantially retains its activity. Purification may be substantial, in which the Spi2A polypeptide or equivalent is the predominant species, or to homogeneity, which purification level would permit accurate degradative sequencing.

Amino acid sequence mutants of Spi2A also are encompassed by the present invention, and are included within the definition of “Spi2A polypeptide equivalent.”

Amino acid sequence mutants of the polypeptide can be substitutional mutants or insertional mutants. Insertional mutants typically involve the addition of material at a non-terminal point in the peptide. This may include the insertion of a few residues; an immunoreactive epitope; or simply a single residue. The added material may be modified, such as by methylation, acetylation, and the like. Alternatively, additional residues may be added to the N-terminal or C-terminal ends of the peptide.

Amino acid substitutions are generally based on the relative similarity of the amino acid side-chain substituents, or example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

Amino acid substitutions are generally based on the relative similarity of the amino acid side-chain substituents, or example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated by reference herein). It is known that certain amino acids may be substituted for other amino acids having a similar hydropathic index or score and still retain a similar biological activity. In making changes based upon the hydropathic index, the substitution of amino acids whose hydropathic indices are within +2 is preferred, those which are within +1 are particularly preferred, and those within +0.5 are even more particularly preferred.

It is understood that an amino acid can be substituted for another having a similar hydrophilicity value and still obtain a biologically equivalent protein. As detailed in U.S. Pat. No. 4,554,101, the following hydrophilicity values have been assigned to amino acid residues: arginine (+3.0); lysine (+3.0); aspartate (+3.0+1); glutamate (+3.0+1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within +2 is preferred, those which are within +1 are particularly preferred, and those within +0.5 are even more particularly preferred.

Certain embodiments of the present invention utilize fusion proteins that are preferentially translocated through biological membranes. In particular, the Spi2A polypeptide, functional Spi2A equivalent, or mutant Spi2A may be fused to a particular protein, polypeptide, or peptide sequence that promotes facilitated intracellular delivery of the fusion protein into the targeted cell. Although any fusion protein with the property of facilitated intracellular delivery is contemplated by the present invention, specific examples include fusion proteins utilizing the HIV TAT sequence (Nagahara et al., 1998), the third helix of the Antennapedia homeodomain (Antp) (Derossi et al., 1994), and the HSV-1 structural protein VP22 (Elliott and O'Hare, 1997).

2. Spi2A-Encoding Polynucleotides

Various aspects of the present invention require polynucleotides encoding an Spi2A polypeptide or an Spi2A polypeptide equivalent. For example, various embodiments include methods for modulating cell death that involve contacting the cell with an expression cassette that includes a promoter that is active in the cell, operably linked to a polynucleotide encoding either an Spi2A polypeptide or an Spi2A polypeptide equivalent. In other embodiments, the invention pertains to methods for treating a subject that include administering to the subject a composition that includes an expression cassette operably inked to a polynucleotide encoding either an Spi2A polypeptide or an Spi2A polypeptide equivalent. In still other embodiments, the invention includes methods of preparing donor granulocytes for delivery to a subject that involve contacting the donor granulocytes with an expression cassette that includes a promoter that is active in the granulocytes, operably linked to a polynucleotide encoding either an Spi2A polypeptide or an Spi2A polypeptide equivalent.

The polynucleotide encoding the full length amino acid sequence of murine Spi2A is provided herein as SEQ ID NO: 1. The polynucleotides according to the present invention may encode an entire Spi2A sequence (for example, the amino acid sequence of SEQ ID NO:2), a functional Spi2A protein domain, an Spi2A polypeptide, or an Spi2A polypeptide equivalent. The polynucleotides may be derived from genomic DNA, i.e., cloned directly from the genome of a particular organism.

In other embodiments, however, the polynucleotides may be complementary DNA (cDNA). cDNA is DNA prepared using messenger RNA (mRNA) as a template. Thus, a cDNA does not contain any interrupted coding sequences and usually contains almost exclusively the coding region(s) for the corresponding protein. In other embodiments, the polynucleotide may be produced synthetically.

It may be advantageous to combine portions of the genomic DNA with cDNA or synthetic sequences to generate specific constructs. For example, where an intron is desired in the ultimate construct, a genomic clone will need to be used. Introns may be derived from other genes in addition to Spi2A. The cDNA or a synthesized polynucleotide may provide more convenient restriction sites for the remaining portion of the construct and, therefore, would be used for the rest of the sequence.

The present invention is not limited to SEQ ID NO:1 (i.e., the polynucleotide encoding murine Spi2A), but includes polynucleotides encoding any Spi2A polypeptide equivalent (discussed above). These polynucleotides encoding Spi2A polypeptide equivalents may be naturally-occurring homologous polynucleotide sequences from other organisms. For example, polynucleotides encoding Spi2A polypeptide equivalents include those polynucleotides encoding the human amino acid functional equivalent sequences previously described (i.e., SEQ ID NO. 3-SEQ ID NO. 9). These sequences are provided by way of example, and are not meant to be a summary of all available Spi2A polypeptide equivalents. A person of ordinary skill in the art would understand that commonly available experimental techniques can be used to identify or synthesize polynucleotides encoding other Spi2A polypeptide equivalents. The present invention also encompasses chemically synthesized mutants of these sequences.

Another kind of sequence variant results from codon variation. Because there are several codons for most of the 20 normal amino acids, many different DNAs can encode the Spi2A. Reference to the following table will allow such variants to be identified.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

Allowing for the degeneracy of the genetic code, sequences that have between about 50% and about 75%, or between about 76% and about 99% of nucleotides that are identical to the nucleotides disclosed herein will be preferred. Sequences that are within the scope of “a polynucleotide encoding a Spi2A polypeptide” or “functional equivalent Spi2A polypeptide” are those that are capable of base-pairing with a polynucleotide segment set forth above under intracellular conditions.

As stated above, the Spi2A encoding sequences may be full length genomic or cDNA copies, or large fragments thereof. The present invention also may employ shorter oligonucleotides of Spi2A. Sequences of 12 bases long should occur only once in the human genome and, therefore, suffice to specify a unique target sequence. Although shorter oligomers are easier to make and increase in vivo accessibility, numerous other factors are involved in determining the specificity of base-pairing. Both binding affinity and sequence specificity of an oligonucleotide to its complementary target increases with increasing length. It is contemplated that oligonucleotides of 8, 9, 10, 11, 12, 13, 14, 15, 16, 17, 18, 19 or 20 base pairs will be used, for example, in the preparation of Spi2A mutants and in PCR reactions.

In certain embodiments, one may wish to employ constructs which include other elements, for example, those which include C-5 propyne pyrimidines. Oligonucleotides which contain C-5 propyne analogues of uridine and cytidine have been shown to bind RNA with high affinity (Wagner et al., 1993).

### B. TARGETED DISEASES AND CONDITIONS

The present invention contemplates methods of treating a subject that includes administering to the subject a composition that includes an Spi2A polypeptide or an Spi2A polypeptide equivalent in a pharmaceutical preparation suitable for delivery to the subject. The subject can be a patient with a disease wherein cell death plays a prominent role in the pathophysiology. The cell death can be by any mechanism. For example, cell death can be the result of apoptosis, necrosis, lysosomal instability, ROS, and abnormal cysteine protease activity.

In a preferred embodiment the Spi2A polypeptides and Spi2A polypeptide equivalents are used to prevent cell death due to apoptosis or necrosis. Necrosis and apoptosis are morphologically distinct forms of cell death that underlie the pathogenesis of all disease. Apoptosis occurs through the activation of an intrinsic cell suicide program to remove seriously damaged, potentially dangerous, infected and unwanted cells. However, an inappropriately activated program can lead to a number of pathological conditions, such as cancer, neurodegenerative disorders, A/DS, autoimmune disorders and viral infections (Turk et al., 2002; Steller, 1995). Necrosis is caused by any noxious stimuli that results in irreversible disruption of cellular homeostatic mechanisms (Kerr et al., 1972). The morphological changes that are associated with necrosis result from the progressive degradative action of enzymes on the lethally injured cells. In terms of pathology, the critical difference between necrosis and apoptosis is that the former does not require the active participation of the cell in it's own demise. In many diseases, tissue injury is caused by both apoptosis and necrosis. Thus, Spi2A polypeptides and Spi2A polypeptide equivalents are used to prevent cell death in a wide range of diseases. In addition, Spi2A and Spi2A equivalents can be used to prevent apoptosis and necrosis of ex vivo normal cells. For instance, these agents can be used to prevent cell death of donor granulocytes during the process of preparation of the granulocytes for transfusion and storage.

Any disease or condition wherein there is an excessive rate of cell death is contemplated. Examples include myocardial infarction (MI), septic shock and liver disease.

1. Myocardial Infarction

Acute MI is caused by coagultive necrosis of myocardiocytes following severe ischemia. Patients with acute MI will be treated by intravenous injection or direct cardiac injection with TAT-Spi2A polypeptides or TAT-Spi2A polypeptide equivalents at the same time they receive thrombolytic therapy to alleviate myocardial ischemia (White and Van der Werf, 1998). This will be optimally within 24 hours after the patient presents so as to protect from coagultaive necrosis and reduce infarct size. The treatment of chronic MI by TAT-Spi2A polypeptides or TAT-Spi2A polypeptide equivalents will follow the same protocol. The response to the agent will be monitored by measuring the reduction in ischemic necrosis of the myocardium. Thus, the serum levels will be monitored by a lowering of myocyte proteins: creatine kinase, troponin I and troponin T (Schoen, 1999). A reduction in infarct size will be verified by at least one of the following echocardiology, radioisotype studies, nuclear magnetic resonance and perfusion scintography.

Standard treatment for MI is to alleviate ischemic coagulative necrosis by restoring blood flow to the myocardium (reperfusion). This causes additional injury through the production of ROS that cause necrosis (Kloner et al., 1998). Administration of TAT-Spi2A polypeptides or TAT-Spi2A polypeptide equivalents as described above may also be used to treat reperfusion injury of myocardial tissue.

2. Septic Shock

Sepsis is caused by the response of inflammatory leukocytes, notably macrophages, to systemic infection with bacteria or fingi. Systemic production of the pro-inflamatory cytokines TNF-α, IL-1 and IL-6 by macrophages give rise to sepsis and septic shock. A critical event is the injury of blood vessels caused by the necrotic and apoptotic death of endothelial cells by TNF-α. This leads to excessive coagulation in blood vessels and a restriction of blood flow to vital organs. If untreated severe sepsis causes cardiovascular collapse and systemic hypoperfusion (septic shock) which leads to the shut down of vital organs and death of the patient. Certain embodiments of the invention pertain to systemic application of TAT-Spi2A polypeptides and TAT-Spi2A polypeptide in the treatment of patients with severe sepsis. For example, the criteria for selecting patients and protocol of administration may be as described for use of the sepsis drug, Xigris (Sollet and Garber, 2002; Laterre and Heiselman, 2002). It is anticipated that this would prevent coagulation of blood in vessels servicing vital organs by protecting endothelial cells from death would prevent ischemic necrosis in organs with impaired blood flow. Response to the agent can be monitored by a resoration in normal blood pressure and diminished patient morbidity.

3. Liver Disease

Hepatic failure and cirrhosis are caused by massive hepatocyte necrosis and apoptosis. There are several causes for hepatocyte necrosis which include fulminant viral hepatitis (with hepatitis A, B, C, D, E and G virus), drugs, chemicals and alcohol (Crawford, 1999). Embodiments of the invention pertain to treatment of hepatic failure and cirrhosis by the administration of Spi2A polypeptides and Spi2A polypeptide equivalents by intravenous injection. The goal of treatment is to reduce hepatocyte necrosis and apoptosis and prevent hepatic failure and cirrhosis. The effect of the agent will be measured by the lowering of serum levels of hepatocyte proteins such as transaminases and a reduction in patient jaundice.

4. Diseases Associated with Abnormal Lysosomal Cysteine Protease Activity

In view of the inventor's discovery that Spi2A polypeptides and Spi2A polypeptide equivalents can be used to inhibit the human cysteine cathespins B, L, V, K and H, the invention can be applied in the treatment of any disease or condition associated with abnormal cysteine protease activity. As previously discussed in this specification, a number of diseases are associated with lysosomal cysteine proteases. Examples include cathepsin K in oestoclasts causing osteoporosis and cathespins K, L and S in inflammatory cells causing emphysema (Turk et al., 2002). Treatment of these conditions would be achieved by the intravenous application of Spi2A polypeptides and Spi2A polypeptide equivalents. Therefore, treatment of any disease associated with lysosomal cysteine proteases is contemplated by the present invention.

5. Method of Preparing Donor Granulocytes

A method of preparing donor granulocytes for delivery to a subject in need of granulocyte donation is also contemplated by the present invention. Current methods of preparing donor granulocytes for transfusion are known to be associated with granulocytes death due to apoptosis (Brach et al. 1992). The present invention is directed at alleviating the apoptotic cell death associated with the preparation and storage of donor granulocytes for transfusion (Leavy et al., 2000). It is anticipated that preservation during storage after 48 hours or more will improve granulocyte function and the clinical efficacy of granulocyte therapy for infection with bacteria and fungi. In particular, the method involves obtaining the donor granulocytes, isolating the donor granulocytes, and then contacting the donor granulocytes with a composition that includes a Spi2A polypeptide or Spi2A equivalent prior to administering the donor granulocytes to a subject in need of the donor granulocytes. The subject in need can be a subject with any disease or condition known to be treated with donor granulocytes. Examples of such diseases and conditions include neutropenia (due to chemotherapy, radiotherapy, myelosuppressive drugs leukemia, idiopathic neutropenia or aplastic anemia (Hubel et al., 2001), neonatal sepsis, and diseases associated with a qualitative abnormality of neutrophils such as chronic granulomatous disease. In particular the invention will be of particular usefulness in the treatment of neutropenia due to dose-intensive chemotherapy, which is amenable to transfusion therapy but not other therapies (Liles et al., 1995).

### C. EXPRESSION CASSETTES

1. Overview

Certain embodiments of the invention pertain to methods utilizing compositions that include an expression cassette. In particular, the methods for modulating cell death in a cell may involve contacting a cell with an Spi2A polypeptide or an Spi2A polypeptide equivalent that further includes an expression cassette. The methods of treating a subject may involve administering to the subject a composition of an Spi2A polypeptide or polypeptide equivalent that includes an expression cassette. In addition, the methods of preparing donor granulocytes for donation to a subject in need may include contacting the donor granulocytes with a composition of an Spi2A polypeptide and an Spi2A polypeptide equivalent that includes an expression cassette. One of skill in the art would understand the techniques relating to use of expression cassettes to deliver polynucleotide sequences to cells or subjects. Particular aspects of these techniques are summarized in this specification. This brief summary is in no way designed to be an exhaustive overview of all available experimental techniques related to expression cassettes since one of skill in the art would already be familiar with these techniques.

Throughout this application, the term “expression cassette” is meant to include any type of genetic construct containing a nucleic acid coding for a gene product in which part or all of the nucleic acid encoding sequence is capable of being transcribed. The transcript may be translated into a protein or polypeptide, but it need not be. Thus, in certain embodiments, expression includes both transcription of a gene and translation of a mRNA into a polypeptide.

In order for the expression cassette to effect expression of a polypeptide, the polynucleotide encoding the polynucleotide will be under the transcriptional control of a promoter. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrase “operatively linked” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence. One of skill in the art would understand how to use a promoter or enhancer to promote expression of an Spi2A polypeptide or Spi2A polypeptide equivalent.

In certain embodiments of the invention, the delivery of an expression cassette in a cell may be identified in vitro or in vivo by including a marker in the expression vector. The marker would result in an identifiable change to the transfected cell permitting easy identification of expression. The selectable marker employed is not believed to be important, so long as it is capable of being expressed along with the polynucleotide of the expression cassette. Examples of selectable markers are well known to one of skill in the art.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). One of skill in the art would be familiar with use of IRES in expression cassettes.

Expression cassettes can include a multiple cloning site (MCS), which is a nucleic acid region that contains multiple restriction enzyme sites, any of which can be used in conjunction with standard recombinant technology to digest the vector. See Carbonelli et. al. (1999); Levenson et al. (1998); Cocea (1997). “Restriction enzyme digestion” refers to catalytic cleavage of a nucleic acid molecule with an enzyme that functions only at specific locations in a nucleic acid molecule. Techniques involving restriction enzymes and ligation reactions are well known to those of skill in the art of recombinant technology.

In expression, one will typically include a polyadenylation signal to effect proper polyadenylation of the transcript. The nature of the polyadenylation signal is not believed to be crucial to the successful practice of the invention, and/or any such sequence may be employed. One of skill in the art would understand how to use these signals to effect proper polyadenylation of the transcript.

In certain embodiments of the present invention, the expression cassette comprises a virus or engineered construct derived from a viral genome. The ability of certain viruses to enter cells via receptor-mediated endocytosis and, in some cases, integrate into the host cell chromosomes, have made them attractive candidates for gene transfer in to mammalian cells. However, because it has been demonstrated that direct uptake of naked DNA, as well as receptor-mediated uptake of DNA complexes, is possible, expression vectors need not be viral but, instead, may be any plasmid, cosmid or phage construct that is capable of supporting expression of encoded genes in mammalian cells, such as pUC or Bluescript™ plasmid series. One of ordinary skill in the art would be familiar with use of viruses as tools to promote expression of the polypeptide.

In certain embodiments of the invention, a treated cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

### D. GENE TRANSFER

1. Viral Vectors

In certain embodiments, the methods and compositions of the invention utilize expression cassettes which includes the Spi2A polypeptide or Spi2A polypeptide equivalent in an expression cassette is carried in a vector. One of ordinary skill in the art would understand use of vectors since these experimental methods are well-known in the art. In particular, techniques using “viral vectors” are well-known in the art. A viral vector is meant to include those constructs containing viral sequences sufficient to (a) support packaging of the expression cassette and (b) to ultimately express a recombinant gene construct that has been cloned therein.

One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors.

Adenoviruses are currently the most commonly used vector for gene transfer in clinical settings. Among the advantages of these viruses is that they are efficient at gene delivery to both nondividing an dividing cells and can be produced in large quantities. The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus et al., 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. A person of ordinary skill in the art would be familiar with experimental methods using adenoviral vectors.

The adenovirus vector may be replication defective, or at least conditionally defective, and the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus growth and manipulation is known to those of skill in the art, and exhibits broad host range in vitro and in vivo. This group of viruses can be obtained in high titers, e.g., 109-1011 plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. A person of ordinary skill in the art would be familiar with well-known techniques that are available to construct a retroviral vector.

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin, et al., 1984; Laughlin, et al., 1986; Lebkowski, et al., 1988; McLaughlin, et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

AAV is a dependent parvovirus in that it requires confection with another virus (either adenovirus or a member of the herpes virus family) to undergo a productive infection in cultured cells (Nuzyczka, 1992). In the absence of confection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et. al., 1991; incorporated herein by reference). A person of ordinary skill in the art would be familiar with techniques available to generate vectors using AAV virus.

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995). A person of ordinary skill in the art would be familiar with well-known techniques for use of HSV as vectors.

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

Other viral vectors may be employed as constructs in the present invention. For example, vectors derived from viruses such as poxvirus may be employed. A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been suggested that VEE may be an extremely useful vector for immunizations (Caley et al., 1997). It is contemplated in the present invention, that VEE virus may be useful in targeting dendritic cells.

A polynucleotide may be housed within a viral vector that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro Roux et al., 1989).

2. Nonviral Vectors

Several non-viral methods for the transfer of expression vectors into cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and Lipofectamine-DNA complex, cell sonication (Fechheimer et al., 1987), gene bombardment using high velocity microprojectiles (Yang et al., 1990), polycations (Bousssif et al., 1995) and receptor-mediated transfection (Wu and Wu, 1987; Wu and Wu, 1988). Some of these techniques may be successfully adapted for in vivo or ex vivo use. A person of ordinary skill in the art would be familiar with the techniques pertaining to use of nonviral vectors, and would understand that other types of nonviral vectors than those disclosed herein are contemplated by the present invention.

In a further embodiment of the invention, the expression cassette may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL). One of ordinary skill in the art would be familiar with techniques utilizing liposomes and lipid formulations.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use subcutaneous, intradermal, intratumoral, or intracranial injection to avoid the toxicity and stability problems associated with cationic lipids in the circulation. The interaction of liposomes and plasma proteins is responsible for the disparity between the efficiency of in vitro (Felgner et al., 1987) and in vivo gene transfer (Zhu et al., 1993; Solodin et al., 1995; Thierry et al., 1995; Tsukarnoto et al., 1995; Aksentijevich et al., 1996).

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Liposomal encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

### E. VACCINES

As used herein, a “vaccine” is an antigenic composition capable of inducing an immune response to the antigen in a cell, tissue or animal (e.g., a human). As used herein, an “antigenic composition” may comprise an antigen (e.g., a peptide or polypeptide), a nucleic acid encoding an antigen (e.g., an antigen expression vector), or a cell expressing or presenting an antigen. In particular embodiments of the present invention, the antigenic composition comprises or encodes all or part of a Spi2A polypeptide or a Spi2A polypeptide equivalent. The antigenic composition may be part of a mixture that comprises one or more additional immunostimulatory agents or nucleic acids encoding such one or more agents. Immunostimulatory agents include, but are not limited to an additional antigen, an immunomodulator, an antigen presenting cell or an adjuvant. In other embodiments, one or more of the additional agent(s) is covalently bonded to the antigen or an immunostimulatory agent, in any combination.

In certain embodiments, an antigenic composition or immunologically functional equivalent, may be used as an effective vaccine in inducing a humoral and/or cell-mediated immune response against a tumor or viral disease in an subject. The vaccines of the present invention can be applied in either the prevention of cancer or viral infection in the subject, or treatment of a cancer or viral disease in the subject. Any type of tumor is contemplated for treatment or prevention using the vaccines of the present invention. For example, the cancer may be breast cancer, lung cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, or leukemia. Similarly, any type of viral disease is contemplated for treatment by the vaccines of the present invention. For example, the viral disease may be HIV, HSV, ADV, or any other viral disease known to those of ordinary skill in the art.

In certain embodiments, the immune response is a long-term immune response involving the development of memory T lymphocytes. Spi2A has been shown to promote the development of long-term immunity in a subject (see Example 3 below). The present invention contemplates one or more antigenic compositions or vaccines for use in both active and passive immunization embodiments.

A vaccine of the present invention may vary in its composition of proteinaceous, nucleic acid and/or cellular components. In a non-limiting example, a nucleic encoding a Spi2A polypeptide or Spi2A polypeptide equivalent might also be formulated with a proteinaceous adjuvant. Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

1. Proteinaceous Antigens

It is understood that an antigenic composition of the present invention may be made by a method that is well known in the art, including but not limited to chemical synthesis by solid phase synthesis and purification away from the other products of the chemical reactions by HPLC, or production by the expression of a nucleic acid sequence (e.g., a DNA sequence) encoding a Spi2A polypeptide or Spi2A polypeptide equivalent in an in vitro translation system or in a living cell. Preferably the antigenic composition is isolated and extensively dialyzed to remove one or more undesired small molecular weight molecules and/or lyophilized for more ready formulation into a desired vehicle. It is further understood that additional amino acids, mutations, chemical modification and such like, if any, that are made in a vaccine component will preferably not substantially interfere with the antibody recognition of the epitopic sequence.

2. Genetic Vaccine Antigens

In certain embodiments, an immune response may be promoted by transfecting or inoculating an animal with a nucleic acid encoding one or more Spi2A polypeptides or one or more Spi2A polypeptide equivalents. One or more cells comprised within a target animal then expresses the sequences encoded by the nucleic acid after administration of the nucleic acid to the animal. Thus, the vaccine may comprise “genetic vaccine” useful for immunization protocols. A vaccine may also be in the form, for example, of a nucleic acid (e.g., a cDNA or an RNA) encoding all or part of the peptide or polypeptide sequence of an antigen. Expression in vivo by the nucleic acid may be, for example, by a plasmid type vector, a viral vector, or a viral/plasmid construct vector.

In preferred aspects, the nucleic acid comprises a coding region that encodes a Spi2A polypeptide or a Spi2A polypeptide equivalent. Of course, the nucleic acid may comprise and/or encode additional sequences, including but not limited to those comprising one or more immunomodulators, adjuvants, or therapeutic agents that can be applied in the treatment of cancer or viral disease. One of ordinary skill in the art would be familiar with techniques for preparation of these nucleic acids for use in the vaccines of the present invention.

3. Cellular Vaccine Antigens

In another embodiment, a cell expressing the antigen may comprise the vaccine. The cell may be isolated from a culture, tissue, organ or organism and administered to an animal as a cellular vaccine. Thus, the present invention contemplates a “cellular vaccine.” The cell may be transfected with a nucleic acid encoding a Spi2A polypeptide or Spi2A polypeptide equivalent. Of course, the cell may also express one or more additional vaccine components, such as immunomodulators, adjuvants, or therapeutic agents that can be applied in the treatment of cancer or an infection, such as a viral infection. A vaccine may comprise all or part of the cell.

### F. PHARMACEUTICAL PREPARATIONS

Pharmaceutical preparations of Spi2A polypeptides and Spi2A polypeptide equivalents for administration to a subject are contemplated by the present invention. In addition, pharmaceutical preparations of Spi2A polypeptides and Spi2A polypeptide equivalents for use in preparing donor granulocytes for administration to a subject in need of donor granulocytes are contemplated by the present invention.

1. Formulations

Any type of pharmaceutical preparation of the Spi2A polypeptide or Spi2A polypeptide equivalent is contemplated by the current invention. One of skill in art would be familiar with the wide range of types of pharmaceutical preparations that are available, and would be familiar with skills needed to generate these pharmaceutical preparations.

In certain embodiments of the present invention, the pharmaceutical preparation will be an aqueous composition. Aqueous compositions of the present invention comprise an effective amount an of Spi2A polypeptide, or an Spi2A polypeptide equivalent, and the like, dissolved or dispersed in a pharmaceutically acceptable carrier or aqueous medium. Aqueous compositions of gene therapy vectors expressing any of the foregoing are also contemplated. The phrases “pharmaceutical preparation suitable for delivery” or “pharmacologically effective” of “pharmaceutically acceptable” refer to molecular entities and compositions that do not produce an adverse, allergic or other untoward reaction when administered to an animal, or a human, as appropriate.

As used herein, “pharmaceutical preparation” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for administration by any known route, such as parenteral administration. The preparation of an aqueous composition containing an active agent of the invention disclosed herein as a component or active ingredient will be known to those of skill in the art in light of the present disclosure.

An agent or substance of the present invention can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. A person of ordinary skill in the art would be familiar with techniques for generation of salt forms. The carrier can also be a solvent or dispersion medium containing, for example, water, ethanol, polyol (for example, glycerol, propylene glycol, and liquid polyethylene glycol, and the like), suitable mixtures thereof, and vegetable oils.

The present invention contemplates Spi2A polypeptides and Spi2A polypeptide equivalents that will be in pharmaceutical preparations that are sterile solutions for intravascular injection or for application by any other route. A person of ordinary skill in the art would be familiar with techniques for generating sterile solutions for injection or application by any other route. Sterile injectable solutions are prepared by incorporating the active compounds in the required amount in the appropriate solvent with various of the other ingredients familiar to a person of skill in the art.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure.

The active agents disclosed herein may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes. One may also use nasal solutions or sprays, aerosols or inhalants in the present invention.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. A person of ordinary skill in the art would be familiar with well-known techniques for preparation of oral formulations. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The use of liposomes and/or nanoparticles is also contemplated for the introduction of the modulator of cell death or gene therapy vectors into host cells. The formation and use of liposomes is generally known to those of skill in the art.

2. Dosage

An effective amount of the therapeutic or preventive agent is determined based on the intended goal, for example inhibition of cell death. The quantity to be administered, both according to number of treatments and dose, depends on the subject to be treated, the state of the subject and the protection desired. Precise amounts of the therapeutic composition also depend on the judgment of the practitioner and are peculiar to each individual.

In certain embodiments, it may be desirable to provide a continuous supply of the therapeutic compositions to the patient. For example, following myocardial infarction a continuous intravascular administration of the therapeutic agent may be administered for a defined period of time. For topical administrations, repeated application would be employed. For various approaches, delayed release formulations could be used that provide limited but constant amounts of the therapeutic agent over an extended period of time. Continuous perfusion of the region of interest (such as the heart, following myocardial infarction) may be preferred. This could be accomplished by catheterization, followed by continuous administration of the therapeutic agent. The administration could be post-operative, such as following coronary artery bypass grafting. To treat diseases such as severe sepsis, MI and liver failure the inventor will administer TAT-Spi2A polypeptide or TAT-Spi2A polypeptide equivalents by intravenous injection. It is anticipated that the diseased organs will be perfused with the agent and upon uptake into the cytoplasm of cells via the TAT peptide Spi2A will protect cell from apoptotic or necrotic death. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the doses are administered.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative, uptake efficiencies. Formulation as a pharmaceutically acceptable composition is discussed above.

3. Tracers to Monitor Gene Expression Following Administration

Certain embodiments of the present invention employ delivery of the Spi2A polypeptide or Spi2A polypeptide to the target area of interest using expression cassettes. For instance, the target area of interest can be a tumor. Because destruction of microscopic foci of cells such as cancer cells cannot be observed, it is important to determine whether the target site has been effectively contacted with the expression cassette. This may be accomplished by identifying cells in which the expression construct is actively producing the desired polypeptide product. It is important, however, to be able to distinguish between the exogenous polypeptide and that present in tumor and nontumor cells in the treatment area. Tagging of the exogenous polypeptide with a tracer element would provide definitive evidence for expression of that molecule and not an endogenous version thereof. Thus, the methods and compositions of the claimed invention may involve tagging of the polypeptide encoded by the expression cassette with a tracer element. A person of ordinary skill in the art would be familiar with these methods of tagging the encoded polypeptide.

4. Secondary Treatment

Certain embodiments of the claimed invention provide for a method of modulating cell death in a subject with cancer. Other embodiments provide for methods of treating a subject with cancer. Treatment of any type of cancer is contemplated by the present invention. Examples of such cancers include breast cancer, lung cancer, prostate cancer, ovarian cancer, brain cancer, liver cancer, prostate cancer, cervical cancer, colon cancer, renal cancer, skin cancer, liver cancer, prostate cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, and leukemia.

A wide variety of cancer therapies, known to one of skill in the art, may be used in combination with the compositions of the claimed invention. Examples of some of the existing cancer therapies and chemotherapeutic agents include radiation therapy, chemotherapy, surgical therapy, immunotherapy, and gene therapy. Examples of other cancer therapies include phototherapy, cryotherapy, toxin therapy, or hormonal therapy.

One of skill in the art would know that this list is not exhaustive of the types of treatment modalities available for cancer and other hyperplastic lesions.

One of skill in the art will recognize the presence and development of other anticancer therapies which can be used in conjugation with the compositions comprising expression cassettes and will further recognize that the use of the secondary therapy of the claimed invention will not be restricted to the agents described below.

In order to increase the effectiveness of a an expression construct encoding a polypeptide that modulates cell death, it may be desirable to combine these compositions with other agents effective in the treatment of malignancies. These compositions would be provided in a combined amount effective to kill or inhibit proliferation of the cell. This process may involve contacting the cells with the expression construct and the agent(s) or second factor(s) at the same time. This may be achieved by contacting the cell with a single composition or pharmacological formulation that includes both agents, or by contacting the cell with two-distinct compositions or formulations, at the same time, wherein one composition includes the expression construct and the other includes the second agent.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### NF-κB Protects from the Lysosomal Pathway of Cell Death

### Materials and Methods

Spi2A mRNA expression. Total RNA (4 μg) was extracted from MEFs after treatment with TNF-α (0.2 ng/ml) (R&D) and cyclohexamide (CHX) (0.1 μg/ml) using Trizol Reagent according to manufacturer's instructions (Invitrogen) and Northern blots prepared using standard procedures (Sambrook et al., 2001). Blots were probed with a [α-³²P] dCTP-hexamer labelled cDNA probe encoding Spi2A (Hampson et al., 1997). Blots were stripped and re-probed with similarly labelled control probes encoding IκBα or GAPDH (De Smaele et al., 2001).

In Spi2A-antisense experiments, the level of Spi2A mRNA was quantitated by real-time PCR using primers and probes specific for Spi2A [forward primer 5′-AAC CAG AGA CCC TGA GGA AGT G-3 (hereinafter SEQ ID NO:12), reverse primer 5′-AAC TTG GGC AGG CGC AG-3′ (hereinafter SEQ ID NO:13), probe 5′-AAG AAC TCT CTG AAG CCC AGG ATG ATA CAT GA-3 (hereinafter SEQ ID NO:14) (Inglis et al., 1991) and the cyclophilin A house keeping control gene (Medhurst et al., 2000) [forward primer 5′-CCA TCA AAC CAT TCC TTC TGT AGC-3′ (hereinafter SEQ ID NO:15), reverse primer 5′-AGC AGA GAT TAC AGG ACA TTG CG-3′ (hereinafter SEQ ID NO:16), probe 5′-CAG GAG AGC GTG CCT ACC CCA TCT G-3 (hereinafter SEQ ID NO:17) (Megabases, Inc). Probes were labelled with the fluorescent reporter dye FAM. Four hours after treatment with CHX (10 μg/ml) and TNF-α (10 ng/ml), RNA was extracted from RelA^{+/+} MEFs using Trizol Reagent (Invitrogen), and then cDNA was generated using Superscript First-Strand Synthesis System for RT-PCR (Invitrogen). Real time PCR reactions were carried out using TaqMan Universal PCR Master Mix according to manufacturer's recommended protocol (PE Applied Biosystems) and analysed on an ABI Prism 7700 Sequence Detection System (PE Applied Biosystems). Data were captured and analyzed using Sequence Detector software (PE Applied Biosystems). The slope of the standard curve describes the efficiency of the real time PCR, which allowed us to ensure that the real time PCR reactions consistently ran at >90% efficiency. The relative concentration of Spi2A RNA was calculated by dividing the concentration of Spi2A RNA by that of the cyclophilin A control gene (Hasel and Sutcliffe, 1990).

Retroviral transduction of MEFs. The cDNA for human RelA (p65) was sub-cloned into the Hpa 1 restriction site of the MIGR1 retroviral vector in the forward orientation (Franzoso et al., 1996; Zhang and Ren, 1998). The Spi2A open reading frame (ORF) was amplified by PCR from cDNA prepared from purified T cells using a forward primer (5′-AGA ATT CGC CAC CAT GGC TGG TGT CT CCC CTG-3; hereinafter SEQ ID NO:18) and reverse primer (5′-TGT GGA TCC TCC CTG TCA AAT CAG GCA GCA TAG CGG AT-3; hereinafter SEQ ID NO:19). These primers introduced 5′ Bam HI and 3 EcoRI restriction sites and mutated the stop codon of Spi2A ORF to facilitate the production of an in frame fusion protein between Spi2A and C-terminal 3×FLAG (22 amino acids) after cloning into the 3×FLAG-CMV-14 expression vector (Sigma-Aldrich). Using the same forward primer and a reverse primer specific for 3×FLAG DNA that introduced an EcoRI restriction site (5′-GTG AAT TCA TCA CTA CTT GTC ATC GT-3; hereinafter SEQ ID NO:20), the Spi2A-3×FLAG ORF was amplified by PCR then sub-cloned into the Eco R1 site of the MIGR1 retroviral vector in the forward or reverse orientations. The MIGR1 retroviral vector directed the expression of RelA, Spi2A-3×FLAG or Spi2A-3×FLAG antisense mRNA as a biscistronic mRNA encoding GFP.

Retrovirus was produced as described previously (Burns et al., 1993). Briefly, the cells of the 293 GP packaging line (4×10⁶) were transiently transfected with MIGR1-Spi2A-3×FLAG DNA (6 μg) and DNA encoding Vesticular Stomatis Virus (VSV) glycoprotein (6 μg) using Lipofectamine PLUS reagent according to manufacturer's instructions (Invitrogen). After 48 and 72 h supernatant containing retrovirus was harvested, filtered and stored at −80° C. until needed. MEFs (1-2×10⁶) were seeded in 6-well plates and transduced with 4 mls of retroviral supernatant containing polybrene (8 μg/ml) by centrifugation (1000 g) for 1 h at room temperature, followed by incubation at 37° C. for 24 h. After 48 h, the transduction efficiency was determined by measuring the percentage of GFP-positive MEFs by FACS, which was routinely 96-98%. Transduced MEFs that were in the top 5% of GFP expression were purified by FACS and cloned.

Fluorescence microscopy. Rel A^{-/-} MEFs were transduced with retrovirus encoding Spi2A-3×FLAG or empty vector and plated in a Chamber-Slide (Lab-Tek, Nalge Nunc) overnight at 10,000 cell per chamber in 10% FCS containing DMEM. Immunofluorescence localization for Spi2A was performed using the anti-FLAG antibody (Sigma). Briefly, the cells were washed three-times with chilled PBS, fixed in 4% paraformaldehyde (PFA)-PBS for 15 minutes at room temperature (RT), permeabilized using 0.5% Triton-X-100 (15 min. at RT) followed by 5 washes with chilled PBS. The slides were blocked with 2% normal mouse serum (NMS) in PBS (45 min at RT) followed by incubation with the biotinylated anti-FLAG antibody (10 μg/ml, 90 min at RT). After washing off the unbound antibody, the slides were incubated with Streptavidin (SA)-Alexa 546 (1 μg/ml, 60 min at RT, Molecular Probes) followed by washes with chilled PBS. Finally the cells were mounted in Vectashield mounting medium (Vector labs) containing DAPI as the nuclear stain. The cells were observed and imaged on a Leica DMIRE2 inverted microscope outfitted with a Photometrics CoolSNAP HQ digital camera (Roper Scientific). Fluorescence images of each of the fluorophores were acquired sequentially using the following Chroma filter cubes; DAPI-cube #31000 (Ex. 340-380 nm, Em. 435-485 nm), and FLAG-cube #41004 (Ex. 535-585, Em. 610-680 nm). Images were acquired and then overlaid using Meta Imaging Series software version 4.6.5 (Universal Imaging Corporation). To determine whether FLAG was distributed throughout the cytoplasm (rather than being bound to the plasma membrane), Z-series of individual cells were captured and deconvolved using MetaMorph. A 3D model was reconstructed from the Z-series, then sliced, and rotated (again, using MetaMorph) to obtain a side view.

Protein expression. Anti-serum specific to Spi2A peptides [peptide 1 (amino acids 406-423) NH₂—(C) NPERSTNFPNGEGASSQR—COOH (hereinafter SEQ ID NO:21); peptide 2 (amino acids 278-294) NH₂—(C) SLQPETLRKWKNSLKPR—COOH (hereinafter SEQ ID NO:22) was raised in rabbits using standard procedures (Coligan et al., 1995). Briefly, two rabbits were immunized with each peptide conjugated to KLH then over a period of 3 months boosted twice with immunogen. Anti-Spi2A antibodies were affinity purified on columns of immunizing peptide and eluted in 3M KSCN then dialysed against PBS.

Detergent extracts from RelA^{-/-} MEFs transduced with control or retrovirus encoding Spi2A were resolved by SDS-PAGE then immunoblotted (25 μg per lane) and probed with either peptide 1 or peptide 2-specific anti-Spi2A antibodies (10 μg/ml), using standard protocols (Coligan et al., 1995). Spi2A was detected after probing with goat-anti-rabbit IgG conjugated to horseradish peroxidase (HRP) (Sigma-Aldrich) at 2 μg/ml and chemilluminescence (ECL-kit, Amersham). Antibodies purified from both rabbits immunized with either peptide 1 or 2 detected Spi2A as a 52 kD protein in extracts from Spi2A cells but not GFP cells. To control for equal loading, blots were stripped and re-probed for actin (42 kD) with anti-actin monoclonal antibody clone ACTN05, (RDI Research Diagnostics, Inc) at 0.5 μg/ml and anti-mouse IgG-HRP (Sigma-Aldrich) at 2 μg/ml.

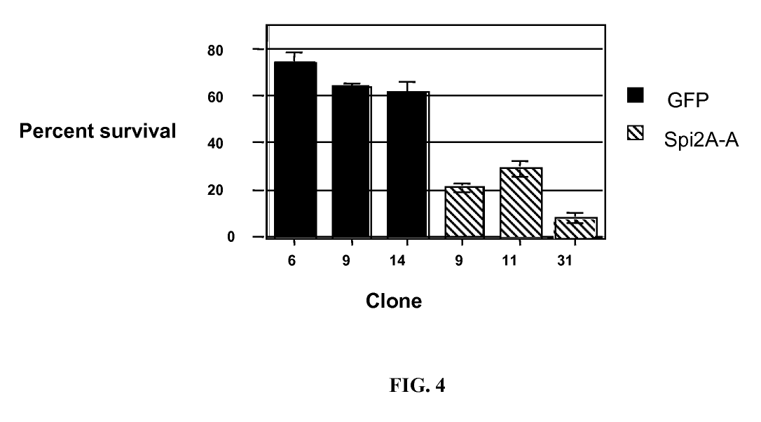

Survival Assays. Although RelA^{-/-} MEFs are markedly more sensitive to TNF-α compared to RelA^{+/+} MEFs, low levels of CHX (0.1 μg/ml) were used in the survival assays. This was to suppress any protective activity of constitutively active non-RelA NF-κB molecules that are present in RelA^{-/-} MEFs (FIG. 4). Thus, RelA^{-/-} MEFs were treated with CHX and TNF-α (R&D) and the number of live GFP-positive adherent cells was counted by flow cytometry after 16 h, if not indicated otherwise. Live cells were defined as those that excluded propidium iodide (PI-negative) and had the appropriate size, as defined by forward and side scatter characteristics (Coligan et al., 1995). For RelA^{+/+} MEFs, TNF-α-cytotoxicity was determined after 16 h with CHX (10 μg/ml), if not indicated otherwise. Cathepsin B activity was inhibited by a 1 h pre-treatment of MEFs with CA-074 Me (30 μM) (Peptide Institute). Complete inhibition of cathepsin B activity was verified by enzyme assay. RelA^{-/-} MEFs were treated with (10-50 μM) sphingosine (Calbiochem) and the number of live GFP-positive adherent cells was counted by flow cytometry after 2 h.

Death effector assays. Death effector pathways were induced in RelA⁴ MEFs by treatment TNF-α (0.2 ng/ml) and CHX (0.1 μg/ml). Assays for executioner proteases (caspases and cathepsin B) were performed on crude cytoplasmic extracts (Stegh et al., 2000). Briefly, MEFs (10⁶) were lysed in 10 mM Tris-Cl, pH 7.5, 100 mM NaCl, 1 mM EDTA, 0.01% Triton X-100 (50 μl) for 30 min on ice then centrifuged at 15,000 g for 30 min at 4° C. and the supernatant recovered. Protein concentration was determined by Lowry assay (DC-protein assay kit, Biorad). Western immunoblots were performed on crude cytosolic extracts (50 μg per lane) using standard protocols and probed with the following antibodies: goat anti-mouse Bid antiserum (1 μg/ml; R&D systems), rabbit anti-human caspase 9 antiserum (2 μg/ml; Cell Signaling Technology), rabbit anti-human caspase 3 antiserum (2 μg/ml; Cell Signaling Technology), mouse anti-human caspase 8 monoclonal antibody clone 12F5 (1 μg/ml; Axxora). The following secondary antibodies were used: anti-goat IgG HRP (0.5 μg/ml; Santa Cruz Technology), anti-rabbit IgG HRP (0.5 μg/ml; Amersham), anti-mouse IgG HRP (0.5 μg/ml; Santa Cruz Technology). Specific proteins were visualized using chemilluminesence (ECL-kit, Amersham).

Colorimetric assays for capases were performed in reaction buffer (50 mM HEPES, pH 7.4, 100 mM NaCl, 10 mM DTT, 1 mM EDTA, 10% glycerol, 0.1% CHAPS) at 37° C. on crude cytoplasmic extracts using p-Nitoaniline (pNA)-labeled substrates (Calbiochem) specific for caspases 3 and 7 (Ac-DEVD-pNA), caspase 8 (Ac-IETD-pNA), caspase 9 (Ac-LEHD-pNA), each at 0.2 mM. Specific activity was determined by subtracting the apparent activity detected after 1 h pre-incubation of extract with the pan-caspase inhibitor Z-VAD.fmk at 50 μM (ICN Biomedicals Inc), then normalizing for the amount of protein. Assays for cathepsin B activity in crude cytoplasmic extracts was performed in reaction buffer (100 mM KHPO₄pH 6.1, 2 mM DTT, 1 mM EDTA) at 37° C. using the Z-RR-pNA substrate, which is specific for cathespin B but not other cysteine cathespins from the lysosome (Barrett and Kirschke, 1981), at 0.4 mM (Calbiochem). Specific activity was determined by subtracting the apparent activity detected after 30 min pre-incubation of extract at 37° C. with the cathespin B inhibitor CA-074 Me at 30 μM (Peptide Institute) then normalizing for the amount of protein.

Mitochondrial membrane potential and ROS production were measured using the fluorescent dyes JC-1 (3 μg/ml) and dihydroethidium (HE) (5 μM)(Molecular Probes) respectively, and flow cytometry according to the manufacturer's instructions.

Protease specificity of Spi2A. RelA^{-/-} MEFs were transduced with retrovirus encoding Spi2A with a C-terminal 3×FLAG epitope tag and Spi2A-3×FLAG purified using a method described previously (Cooley et al., 1998). Briefly, cells (3×10⁹) were lysed and Spi2A-3×FLAG (75 μg) purified by batch Q-Fast Flow ion-exchange chromatography (Pharmacia Biotech) after elution at 160-220 mM NaCl followed by anti-FLAG antibody columns, performed according to manufacturer's instructions (Sigma-Aldrich). Spi2A-3×FLAG was dialysed into PBS and stored as aliquots at −80° C. until needed.

Proteases were purchases from the manufacturers (Calbiochem or Athens Research and Technology) except granzymes A and B which were purified as described (Hanna et al., 1993) and cathespin V and K, which were purified as described (Bromme et al., 1999; Linnevers et al., 1997). Proteases (20 nM) were incubated in the appropriate assay buffer with Spi2A at 200 nM (at least 10-fold excess of inhibitor to maintain pseudo-first order conditions) for 1 h at 37° C. Control samples included only the enzyme, without the inhibitor. At the end of 1 h protease activity was assayed. For serine proteases the following substrates (Calbiochem™) were used at 1 mM (Al-Khunaizi et al., 2002; Cooley et al., 2001): human cathepsin G-Suc-AAPF-pNA; human elastase-MeOSuc-AAPV-pNA in assay buffer (20 mM Tris-HCl, pH 7.4, 500 mM NaCl, 0.1% PEG); human granzyme B-IETD-pNA; human granzyme A-BLT-pNA. For cysteine cathepsins, the following substrates (Molecular Probes) were used at 5 μM: human cathepsin B, L, K and V, (Z-FR)₂-R110; human cathepsin H, (Z-PR)₂-R110 in assay buffer (50 mM NaAc pH 5.4, 4 mM DTT, 1 mM EDTA)(Al-Khunaizi et al., 2002). Substrate hydrolysis was measured in a fluorescence microtiter plate reader (Spectramax Gemini XS, Molecular Devices). Percentage inhibition was calculated from the residual enzyme activity compared to no Spi2A controls. Incubation with alkaline phosphatase tagged with C-terminal 3×FLAG (Sigma-Aldrich) under the same conditions had no effect on protease activity.

Transduction of Rel A^{-/-} MEFs with Retrovirus Encoding Rel A Results in the expression of Rel A. Over expression of members of the Rel family of transcription factors are known to inhibit cell division (Bash et al., 1997). Therefore, to avoid over expression of Rel A at levels greater than wild-type, RelA^{-/-} MEFs were analyzed only 24 h after transduction. Rel A was detected in Rel A^{-/-} MEFs 24 h after transduction with retrovirus encoding Rel A by probing immunoblots (50 μg per lane) with rabbit anti-Rel A (1 μg/ml) (Sressgen Biotechnologies).

Spi2A does not affect NF-κB activation. The possibility that Spi2A protects from TNF-α-induced apoptosis by directly activating NF-κB was examined. Therefore, using electrophoretic mobility shift assays (EMSAs), studies were conducted to determine whether Spi2A itself promotes NF-κB activation in nuclei from Rel A^{-/-} MEFs transduced with Spi2A or whether the abrogation of Spi2A mRNA up-regulation by Spi2A antisense message inhibits NF-κB activation in Rel A^{+/+} MEFs. In Rel A^{-/-} MEFs, super shift analysis of nuclear fractions with specific antibodies revealed the presence of p50/p50 homodimers and heterodimers of p50 and Rel-related proteins other than Rel A. As has been noted previously, the p50/Rel heterodimers but not the p50/p50 homodimers were induced by TNF-α (Franzoso et al., 1993; Franzoso et al., 1992). Importantly, the expression of Spi2A did not affect the expression of these NF-κB complexes. In Rel A^{+/+} MEFs, super-shift analysis confirmed the induction of p50/Rel A by TNF-α. The inhibition of Spi2A mRNA induction in Spi2A-A cells did not abrogate the induction of NF-κB by TNF-α. It was concluded that Spi2A does not directly affect the activation of NF-κB transcription factors following stimulation with TNF-α. Thus, the anti-apoptotic activity of Spi2A is unlikely to be mediated through the modulation of NF-κB activation.

Kinetics of cathepsin B inhibition by Spi2A. The second-order association rate constant for the inhibition of cathepsin B by Spi2A was measured by following the continuous hydrolysis of the cathepsin B substrate, (Z-FR) 2-R110, in presence and in absence of a 10-fold excess of the inhibitor (Pseudo-first order conditions). Briefly, 20 nM cathepsin B was added to 5 μM substrate, in presence or in absence of 200 or 400 nM (10- or 20-fold excess respectively) of Spi2A in activation buffer. Reactions were performed in 96-well microtitre plates. The final reaction volume was 200 μl. The rate of substrate hydrolysis was continuously monitored for 3 min. The dead time for the measurement (the time between the addition of the enzyme to the substrate (with or without inhibitor) and the first spectrophotometric measurement was ˜30 s.

The inhibitory reaction was too fast to be measured using our conventional spectrophotometer. The enzyme was completely inhibited, within the dead time of the measurement. This indicated that the second-order association rate constant approached diffusion limited rates (>10⁶M⁻¹ s⁻¹) TNF-α disrupts the pH of lysosomes. Lysosome internal pH was measured by staining with the weak basic, lysosomotropic dye—acridine orange (AO)— and flow cytometry (Zhao et al., 2000). Increase in lysosomal pH results a corresponding decrease in AO red fluorescence and the appearance of AO-low cells. It was shown that TNF-R1 cross-linking results in the appearance of AO-low cells in Rel A^{-/-} MEFs transduced with control retrovirus (GFP) (Supplementary FIG. 4). Rel A^{-/-} MEFs transduced with Spi2A exhibited the appearance of less AO-low cells. The appearance of AO-low cells can be interpreted as lysosomal rupture, and so it is possible that Spi2A in some way protects lysosomes from damage induced by TNF-α.

### Results

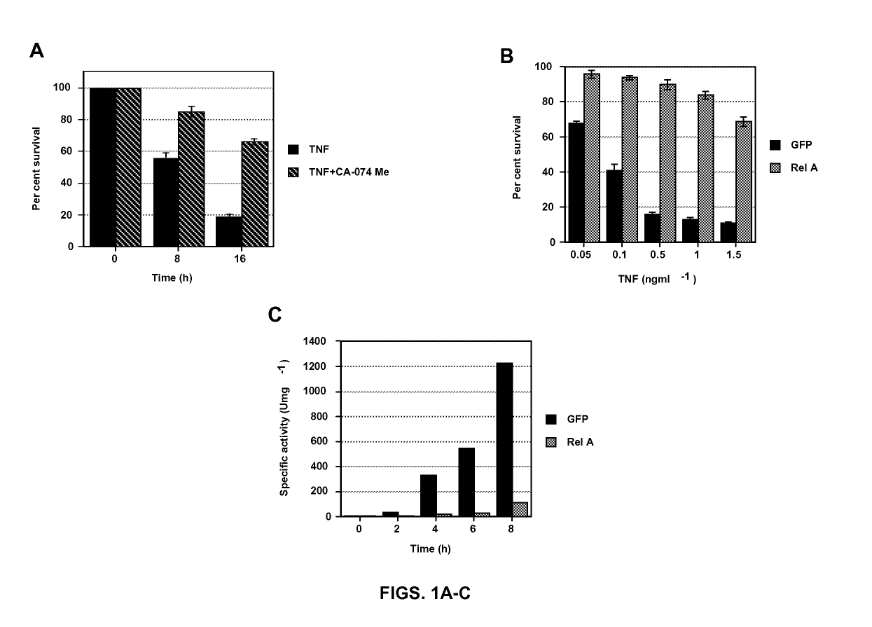

NF-κB antagonizes the lysosomal pathway of cell death. NF-κB protects cells from TNF-α-mediated death through the up-regulation of protective genes, which inhibit the apoptotic cascade at several different points. A role for cathepsin B has been demonstrated in the TNF-R-induced death of several types of tumor cells using specific inhibitors of cathepsin B, such as CA-074 Me (Foghsgaard et al., 2001). The complete inhibition of cathepsin B activity by CA-074 Me (30 μM) protected RelA^{-/-} MEFs from TNF-α-induced death (FIG. 1A). Therefore, cathepsin B activity contributes to the susceptibility of RelA^{-/-} MEFs to TNF-α-induced apoptosis.

Studies in primary and tumor cells have demonstrated that activation of TNF-R1 results in the release of cathespin B from the lysosome into the cytoplasm where it triggers apoptosis (Foghsgaard et al., 2001; Guicciardi et al., 2000; Wemeburg et al., 2002). Using RelA^{-/-} MEFs, the effect of RelA complementation on the induction of cytosolic cathespin B activity after TNF-α treatment was examined. RelA^{-/-} MEFs were transduced with retrovirus encoding RelA on a polycistronic mRNA encoding GFP (Zhang and Ren, 1998). As has been shown before, expression of RelA in RelA^{-/-} MEFs restored NF-κB function and gave complete protection from TNF-α-cytotoxicity (FIG. 1B) (Beg and Baltimore, 1996). The influence of NF-κB/RelA on the induction of cathespin B activity in the cytosol after treatment with TNF-α was next examined. An increase in cathepsin B activity of cytosolic extracts from control RelA^{-/-} MEFs as early as two hours after treatment with TNF-α was observed, which then increased with time (FIG. 1C). In contrast, transduction with RelA extinguished cathepsin B activity in the cytoplasm of RelA^{-/-} MEFs for as long as eight hours after treatment with TNF-α (FIG. 1C). Thus, NF-κB may up-regulate genes that inhibit cathepsin B activity in the cytosol.

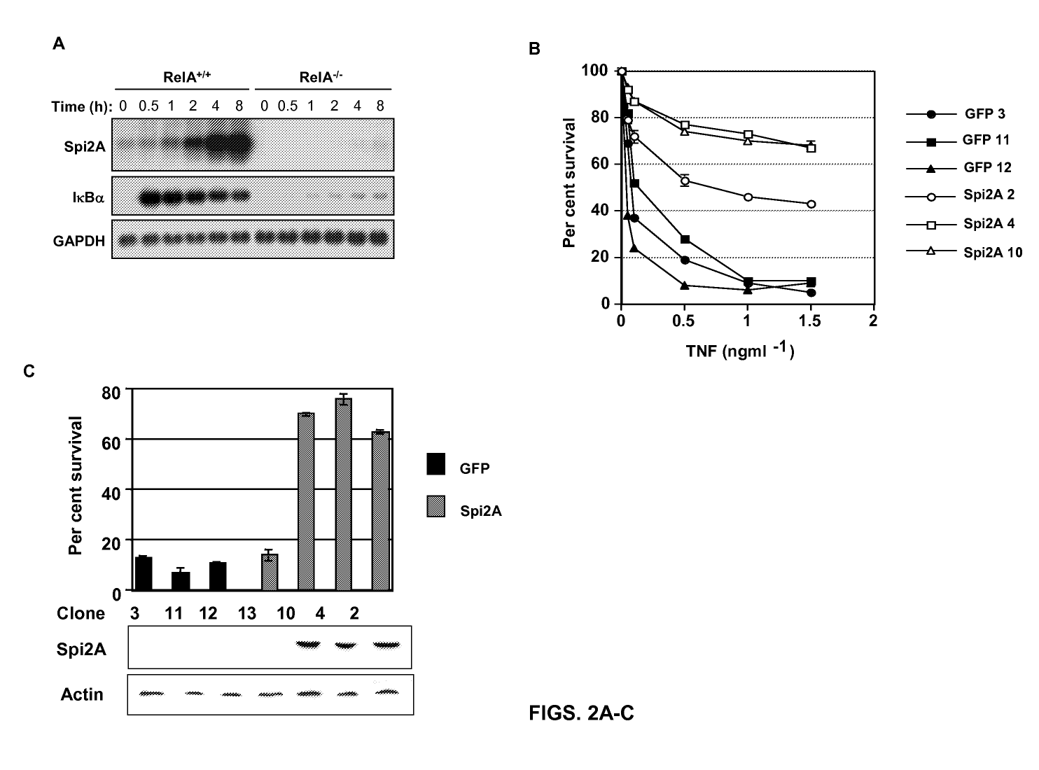

Induction of Spi2A by NF-κB protects from TNF-α-mediated cell death. The transcription of Spi2A is induced by inflammatory stimulation and depends on NF-κ-binding (Hampson et al., 1997; Hampson et al., 2001; Inglis et al., 1991). Initially, studies were conducted to determine whether Spi2A was a physiologic target of NF-κB. Spi2A mRNA (2.3 kb) was strongly induced by TNF-α in RelA^{+/+} MEFs, but this induction was completely abolished in NF-κB/RelA^{-/-} MEFs (Beg and Baltimore, 1996) (FIG. 2A). While dramatic, the induction of Spi2A expression occurred with slower kinetics compared to the expression of ikbα, a known target of NF-κB (De Smaele et al., 2001). These results indicate that Spi2A is a physiological target of NF-κB.

The control of cell survival is critically dependent on the induction of protective genes by NF-aB transcription factors (Karin and Lin, 2002). Studies were conducted to examine whether Spi2A can protect RelA^{-/-} MEFs from TNF-α-induced death. RelA^{-/-} MEFs were transduced with retrovirus encoding Spi2A on a polycistronic mRNA with the GFP gene (Zhang and Ren, 1998). Cells from stable clones transduced with Spi2A (Spi2A cells) exhibited markedly improved survival against TNF-α, Whereas cloned cells transduced with vector alone (GFP cells) did not (FIG. 2B). Protection of RelA^{-/-} MEFs from TNF-α correlated with the expression of Spi2A protein (FIG. 2C). At low concentrations of TNF-α protection by Spi2A was virtually complete (FIG. 2B, see 0.5 ng/ml TNF-α) and was dramatic even after 16 hours at high concentrations, indicating that Spi2A can temporarily substitute for NF-κB complexes in inhibiting TNF-α-induced apoptosis.

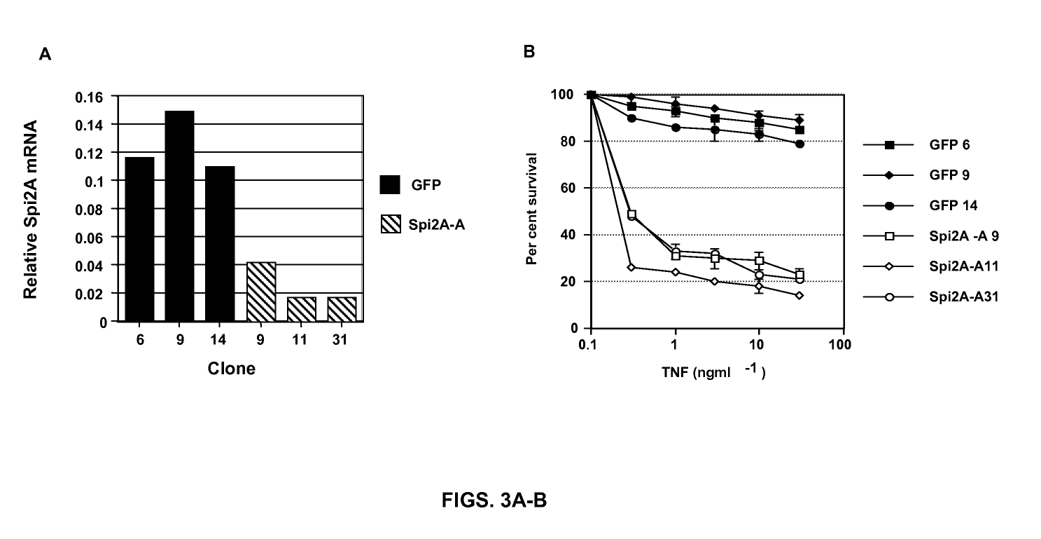

To verify that cyto-protection mediated by Spi2A was not due to over-expression, we generated wild-type (RelA^{+/+}) MEFs expressing Spi2A in an antisense orientation (Spi2A-A cells). After treatment with TNF-α, analysis by real-time PCR revealed that the up-regulation of endogenous Spi2A mRNA was abrogated in stable clones of Spi2A-A cells (Medhurst et al., 2000) (FIG. 3A). Despite their ability to activate NF-κB, Spi2A-A cells exhibited a marked susceptibility to TNF-α-induced cell death (FIG. 3B). The sensitivity of Spi2-A cells to TNF-α was also observed in the absence of cyclohexamide (CHX), indicating that TNF-α-cytotoxicity was not due to an inhibition of protein synthesis in Rel A^{+/+} MEFs (FIG. 4). Thus, Spi2A is required to antagonize TNF-α-induced apoptosis, and protection from death is a physiological function of Spi2A.

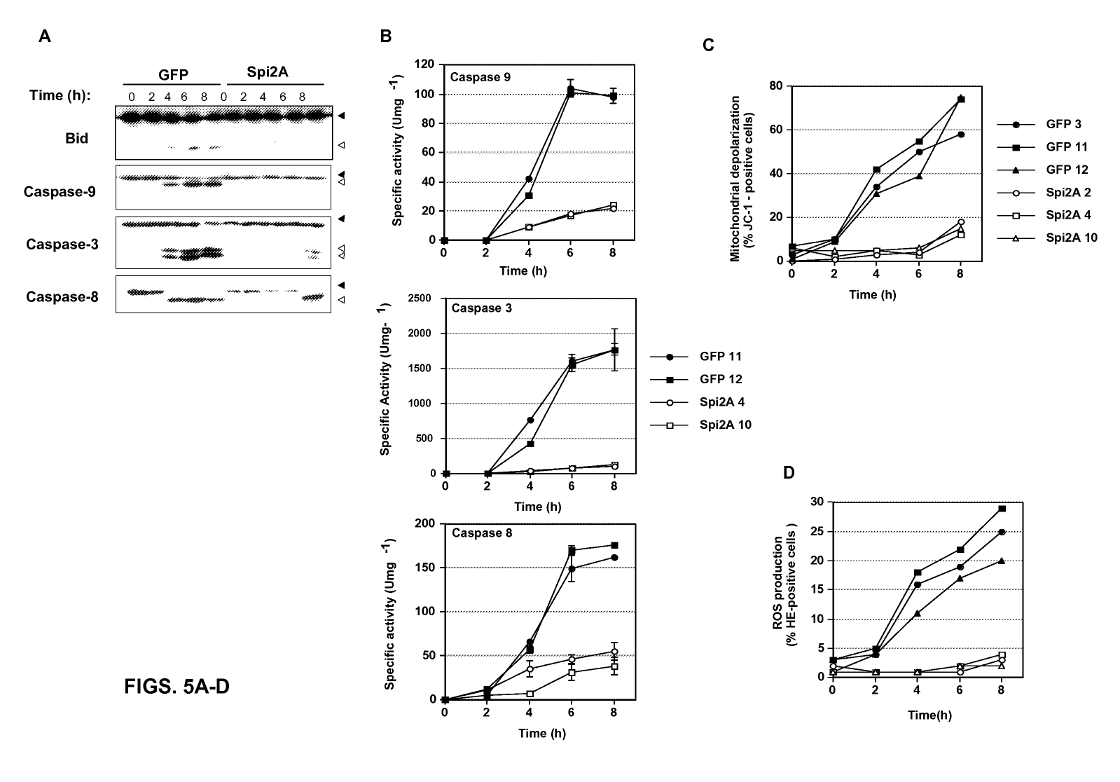

Spi2A protects from apoptosis. NF-κB protects cells from death induced by TNF-α by up-regulating the expression of genes which antagonize the mitochondrial pathway of apoptosis (Baldwin, 2001; Beg and Baltimore, 1996). Given the ability of Spi2A to substitute for NF-κB complexes in protecting from TNF-α, studies were conducted to determine whether Spi2A could inhibit the mitochondrial pathway of apoptosis. In RelA^{-/-} MEFs, TNF-α activation of caspase-8, 9 and 3, and the pro-apoptotic Bcl-2 family member Bid, was assessed by western blots (FIG. 5A), and in vitro enzyme assays Budihardjo et al., 1999; Stegh et al., 2000) (FIG. 5B). Remarkably, the activation of both apical and executioner caspases, as well as Bid, was suppressed in RelA⁴ MEFs that expressed high levels of Spi2A. In these cells mitochondrial depolarization—a key indicator of apoptosis—was virtually abrogated by Spi2A (Budihardjo et al., 1999) (FIG. 5C). Importantly, Spi2A also suppressed the production of reactive oxygen species (ROS), which mediate TNF-α-cytotoxicity (Goossens et al., 1995) (FIG. 5D). Thus, Spi2A abrogates TNF-α-induced caspase activation, mitochondrial depolarization and ROS production in NF-κB null cells, thereby recapitulating the effects of the transcription factor on apoptosis (Wang et al, 1998).

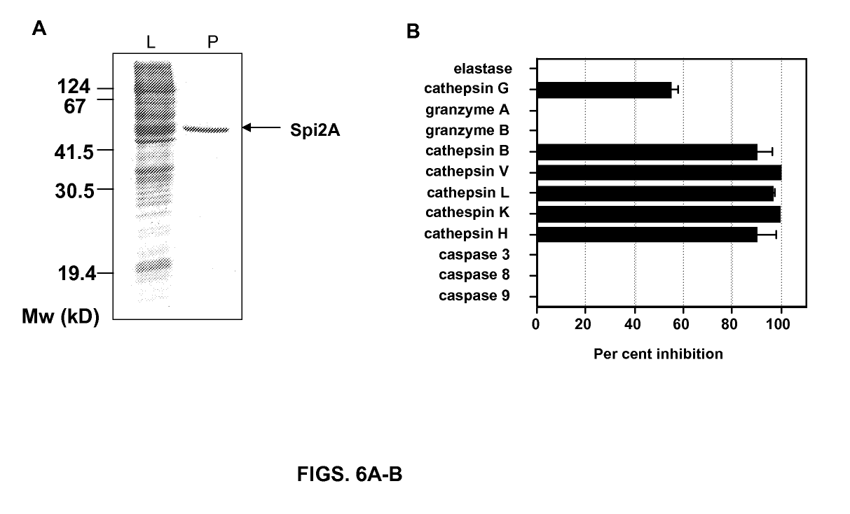

Spi2A inhibits lysosomal cysteine cathepsins. To determine the mechanism by which Spi2A antagonized apoptosis, studies were conducted to examine the protease specificity of Spi2A in vitro. Spi2A was purified from RelA^{-/-} MEFs transduced with retrovirus encoding epitope-tagged Spi2A (Cooley et al, 2001) (FIG. 6A). Spi2A inhibited both serine and cysteine proteases, similar to the serpin, SQN-5 (Al-Khunaizi et al, 2002). Spi2A inhibited the chymotrypsin-like, serine protease cathepsin G, but not elastase or either granzyme B or granzyme A (FIG. 6B). The specificity of Spi2A for cysteine proteases extended to all of the lysosomal, papain-like proteases that were examined—cathepsin B, V, L, K and H. Spi2A inhibited cathespin B with a rate constant k of >10⁶ M⁻¹ s⁻¹, and so is likely to be a physiologically relevant inhibitor in vivo (Silverman et al., 2001). However, the inhibitory effects of Spi2A did not extend to any of the caspases tested (3, 8 or 9) (FIG. 6B). Thus, Spi2A is a cross-class specific inhibitor of both serine proteases and lysosomal cysteine cathepsins.

Spi2A localizes to the cytoplasm and nucleus. Spi2A is an unusual member of the chymotrypsin-like family of serpins in that it lacks a secretory signal sequence and so is likely to be located intracellularly (Hampson et al, 1997). To further examine of Spi2A in protection from TNF-α-induced apoptosis we first determined the intracellular location of FLAG-tagged Spi2A in stably transduced Rel A^{-/-} MEFs (FIG. 2B). Immunofluorescence studies revealed staining with anti-FLAG antibodies in the cytoplasm and nucleus. Z-section analysis confirmed uniform distribution of anti-FLAG staining throughout the cytoplasm rather than in the plasma membrane. It was concluded that Spi2A resides in the cytoplasm and nucleus. The nucleo-cytoplasmic localization of Spi2A revealed by these studies is concordant with findings of others with macrophage cell lines and COS cells using Spi2A anti-sera in immuofluorescence studies (Morris et al, 2002). Localization in the cytoplasm raises the possibility that Spi2A may protect from apoptosis through the inhibition of cathepsin activity after release from the lysosome (FIG. 1C).

Spi2A antagonizes the lysosomal pathway of cell death. The up-regulation of Spi2A by NF-κB protects cells from apoptosis following ligation of TNF-R1 (FIG. 2). Spi2A can inhibit cathepsin B in vitro (FIG. 6B), and is located in the cytosol. Therefore, the induction of Spi2A and inhibition of cathepsin B after it is released into the cytoplasm may be a mechanism by which NF-κB antagonizes the lysosomal pathway of cell death (FIG. 1).

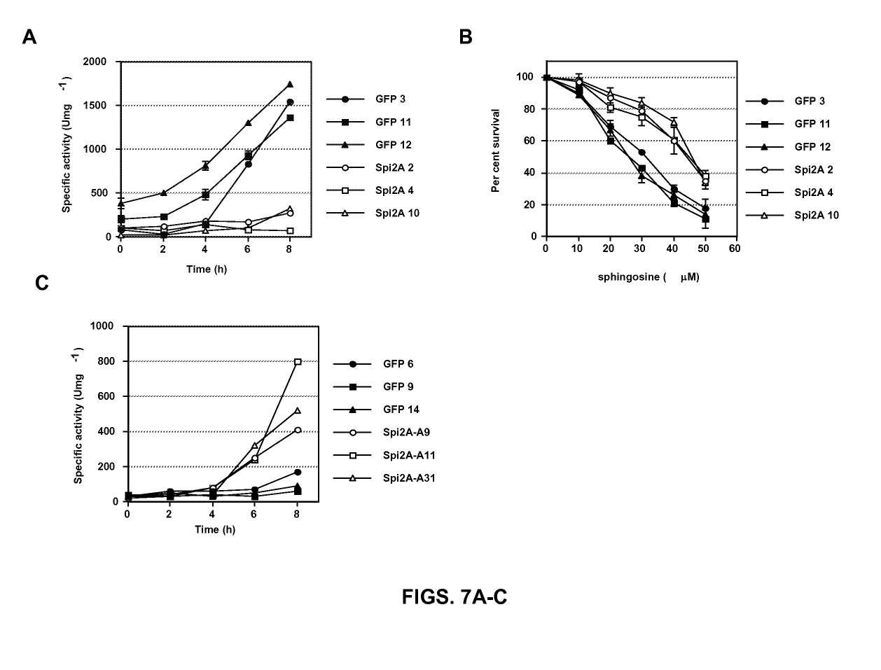

As was observed with Rel A complementation (FIG. 1B), Spi2A inhibited the induction of cytosolic cathepsin B activity, after treatment of Rel A^{-/-} MEFs with TNF-α (FIG. 7A). Direct treatment of cells with sphingosine causes the release of cathespin B from the lysosome and the induction of apoptosis (Foghsgaard et al., 2001; Kagedal et al., 2001; Werneburg et al., 2002). Consistent with a role in protecting from lysosome-mediated apoptosis, Spi2A could protect Rel A^{-/-} MEFs from death after treatment with sphingosine (FIG. 7B). Overall, these results indicate that Spi2A abrogates TNF-α-induced activation of cytoplasmic cathepsin B in NF-κB null cells, thereby recapitulating the effects of the transcription factor on the lysosomal pathway of apoptosis.

Importantly, the inhibition of endogenous Spi2A mRNA expression by antisense Spi2A resulted in the induction of cytoplasmic cathepsin B activity after treatment of RelA^{+/+} MEFs with TNF-α (FIG. 7C). These results indicate that the inhibition of cathepsin B activity in the cytosol by Spi2A is a physiologically relevant mechanism by which NF-κB protects cells from the lysosomal pathway of apoptosis.

### Example 2

### Spi2A Inhibits Caspase-Independent Cell Death

Materials and Methods

TNF-α death assays. NIH3T3 cells were transduced with MIGR1 retrovirus (Zhang and Ren, 1998) encoding either GFP alone, or Spi2A in the forward (sense) or reverse (antisense) orientation and stable clones generated, as described previously (Liu et al., 2003). Cells were treated with TNF-α (R&D) and after 16 h the number of live GFP-positive adherent cells were counted by flow cytometry (Liu et al., 2003). Live cells were defined as those that excluded propidium iodide (PI-negative) and had the appropriate size, as defined by forward and side light scatter characteristics. Caspase activity was inhibited by pre-treatment of cells or extracts for 1 h with Z-VAD.fmk (ICN Biomedicals Inc; 50 μM). Complete inhibition of caspase activity was verified by enzyme assay (Liu et al., 2003). In anti-sense experiments the level of Spi2A mRNA was quantitated by real-time PCR using primers and probes specific for Spi2A (Inglis et al., 1991) and cyclofilin A control mRNA (Medhurst et al., 2000), 4 h after treatment with Z-VAD.fmk (50 mM) and TNF-α (10 ng/ml), as described previously (Liu et al., 2003).

Death effector assays. Death effector pathways were induced in by treatment of NIH3T3 cells with TNF-α (10 μg/ml) and Z-VAD.fmk (50 μM). Colorimetric assays for cathepsin B were performed on crude cytoplasmic extracts (Stegh et al., 2000). Briefly, NIH3T3 cells (10⁶) were lysed in 10 mM Tris Cl, pH 7.5, 100 mM NaCl, 1 mM EDTA, 0.01% Triton X-100 (50 μl) for 30 min on ice then centrifuged at 15,000×g for 30 min at 4° C. and the supernatant recovered. Protein concentration was determined by Lowry assay (DC-protein assay kit, Biorad). Cathepsin B was assayed in reaction buffer using the p-Nitoaniline (pNA)-labeled substrate Z-RR-p NA (Calbiochem) (Barrett and Kirschke, 1981). Specific activity was determined by subtracting the apparent activity detected in the presence of the cathepsins B inhibitor CA074-Me (30 μM) inhibitor (Peptide Institute) (Liu et al., 2003). Mitochondrial membrane potential and ROS production was measured using the fluorescent dyes JC-1 (3 μg/ml) and dihydroethidium (HE) (5 μM) (Molecular Probes) respectively, and flow cytometry according to the manufacturer's instructions.

Results

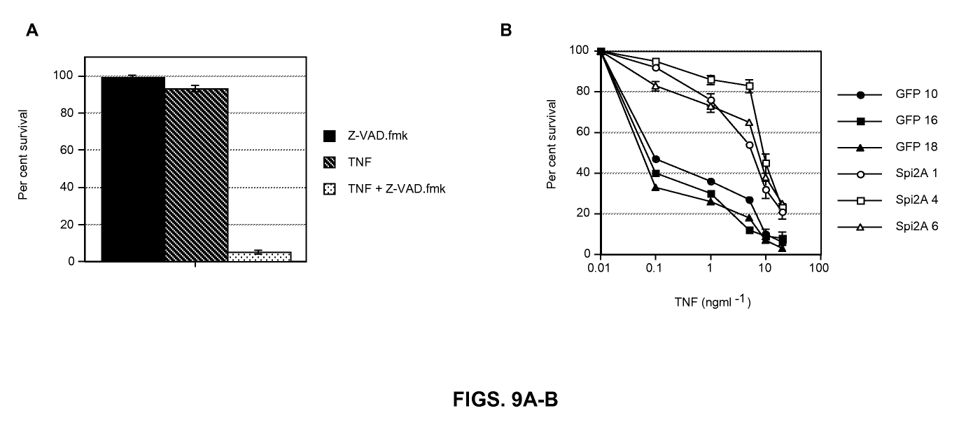

Spi2A protects from caspase-independent PCD. Complete inhibition of caspase activity by Z-VAD.fmk can sensitise normally resistant cells with wild-type levels of NF-κB to TNF-α-induced PCD (Vercammen et al., 1998; Khwaja and Tatton, 1999). In FIG. 9A, as expected (Khwaja and Tatton, 1999), complete inhibition of caspase activity by Z-VAD.fmk sensitized NIH 3T3 fibroblasts to PCD by TNF-α was shown.

Lysosomal cathepsins, such as cathepsin B, can induce PCD in the absence of caspase activity (Foghsgaard et al., 2001). This raises the possibility that because Spi2A is a potent inhibitor of cathepsin B, it may protect against a caspase-independent program of cell death (Bomer and Monney, 1999). To address this, NIH 3T3 cells were transduced with retrovirus encoding Spi2A (Spi2A cells) on a polycistronic mRNA with green fluorescent protein (GFP) and stable clones which express high levels of Spi2A generated (Liu et al., 2003; Zhang and Ren, 1998). In the absence of caspase activity, Spi2A cells exhibited markedly improved survival against TNF-α, compared to cloned cells transduced with GFP alone (GFP cells) (FIG. 9B). Therefore, Spi2A can protect against caspase-independent PCD.

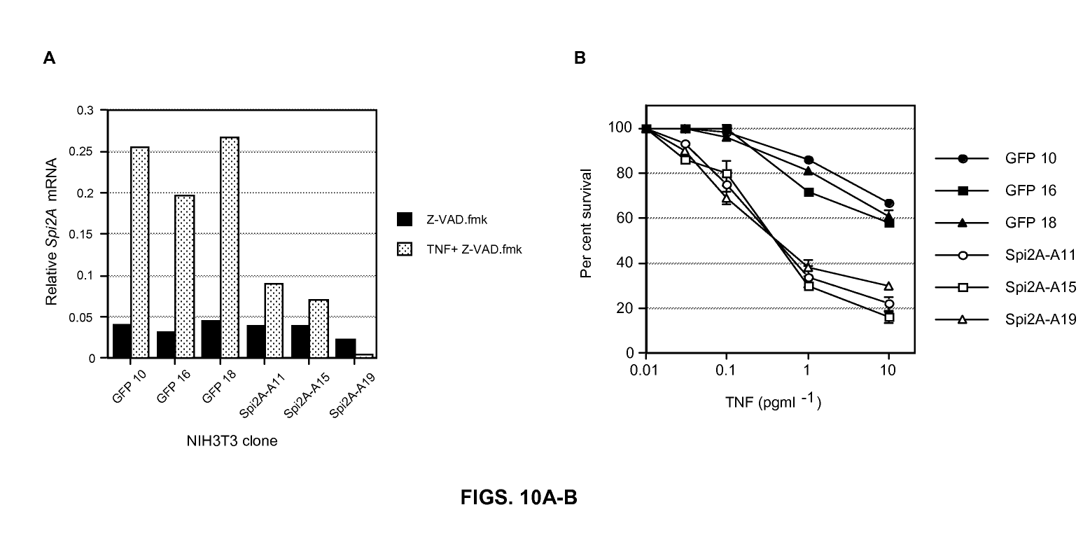

Spi2A is a physiological inhibitor of caspase-independent PCD. To verify that cyto-protection from caspase-independent PCD mediated by Spi2A was not due to over expression, clones of NIH3T3 cells expressing Spi2A in an anti-sense orientation (Spi2A-A cells) were generated (Liu et al., 2003). It has been shown that prior treatment with TNF-α induces the expression of Spi2A in an NF-κB-dependent manner (Liu et al., 2003). As expected, real-time PCR revealed that in the presence of Z-VAD.fmk, treatment with TNF-α resulted in the up-regulation of Spi2A mRNA in control NIH3T3 cells (Medhurst et al., 2000) (FIG. 10A). Importantly, after treatment with TNF-α the up-regulation of endogenous Spi2A mRNA was abrogated in stable clones of Spi2A-A cells (FIG. 10A).

In the absence of caspase activity, the inhibition of endogenous Spi2A mRNA expression by anti-sense message resulted in a marked increase in the susceptibility of cells to TNF-α-induced PCD (FIG. 10B). It has been previously shown that Spi2A has no direct effect on NF-κB activation, therefore is unlikely that the knock-down in Spi2A expression increased PCD by impairing NF-κB function (Liu et al., 2003). Thus, Spi2A is required to antagonize TNF-α-induced PCD in the absence of caspase activity.

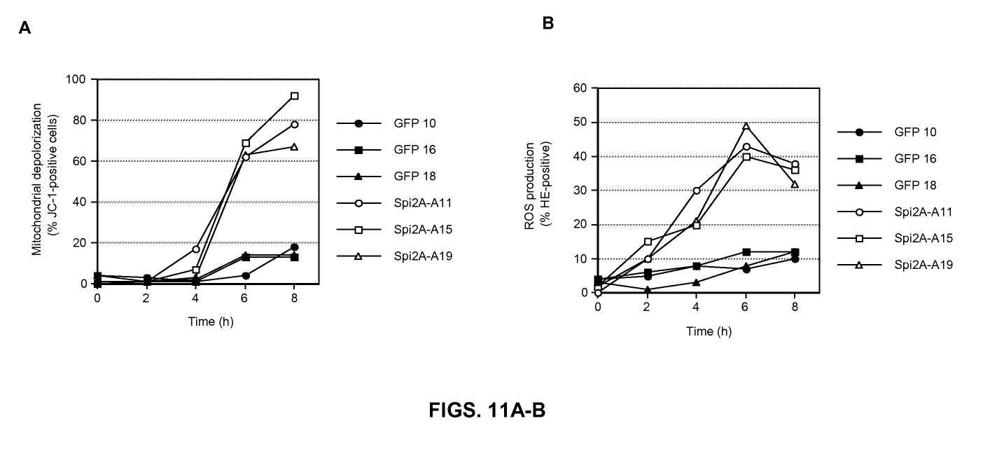

Spi2A suppresses mitochondrial pathways of PCD in the absence of caspase activity. The permeabilization of the outer membrane of the mitochondrion is central to most caspase-independent death programs (Jaattela and Tschopp, 2003). One important consequence of damaged mitochondria is the release of reactive oxygen species (ROS), which are thought to be particularly important in mediating TNF-α cytotoxicity (Goossens et al., 1995). Given the ability of Spi2A to protect from caspase-independent PCD, studies were conducted to determine whether Spi2A could protect cells from mitochondrial depolorization and ROS production. The knock-down in Spi2A expression resulted in the onset of mitochondrial depolorization (FIG. 11A) and ROS production (FIG. 11B) after treatment of Spi2A-A cells with Z-VAD.fmk and TNF-α. Therefore, these results indicate that Spi2A is a physiological inhibitor of caspase-independent mechanisms of PCD.

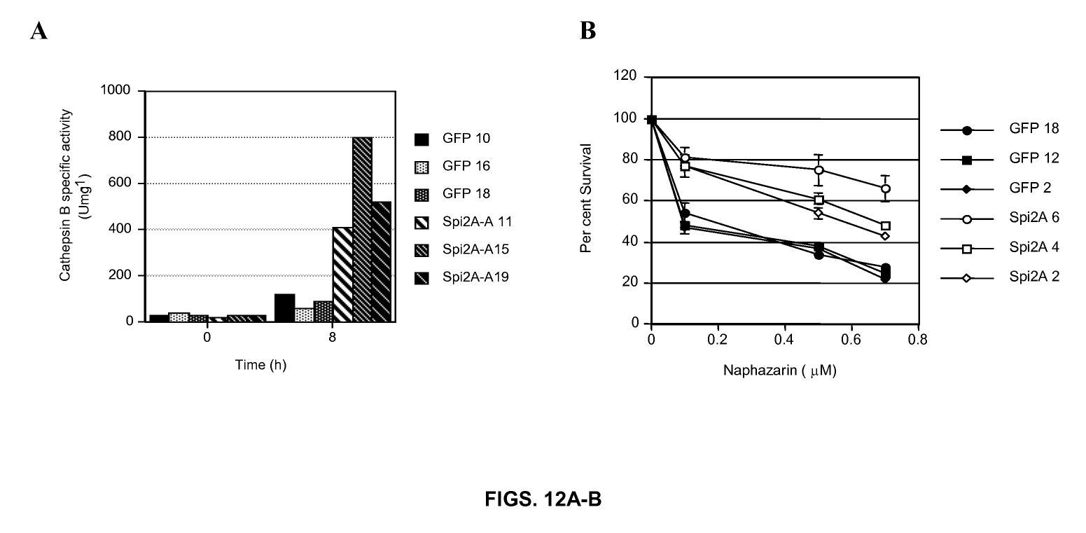

Spi2A is a physiological inhibitor of the lysosomal pathway of death in the absence of caspase activity. Cysteine cathepsins, notably cathepsin B, are potent inducers of both caspase-dependent and caspase-independent PCD (Guicciardi et al., 2000; Foghsgaard et al., 2001; Liu et al., 2003). Spi2A is located in the cytoplasm and so can protect from caspase-dependent apoptosis by suppressing cytoplasmic cathepsin B activity after it is released from the lysosome (Liu et al., 2003). Studies were conducted to determine if this mechanism of cyto-protection by Spi2A extends to the inhibition of caspase-independent PCD. In the absence of caspase activity, the inhibition of endogenous Spi2A mRNA expression by antisense Spi2A resulted in the induction of cytoplasmic cathepsin B activity after treatment of NIH3T3 cells with TNF-α (FIG. 12A). Thus, the inhibition of cytosolic cathepsin B by Spi2A is a physiologically relevant mechanism by which Spi2A blocks the lysosomal pathway of cell death in the absence of caspase activity.

Spi2A protects NIH3T3 cells from death due to reactive oxygen species. NIH3T3 fibroblasts from independent clones harboring control retrovirus (GFP clones #, 18, 12 and 2) or one expressing Spi2A (Spi2A clones# 6, 4 and 2) were incubated with Naphazarin—a known initiator of Reactive Oxygen Species (ROS). After 16 hours, the percentage of live cells was determined by flow cytometry as described in Liu et al., 2003. A significantly increased survival of cells from all three clones expressing Spi2A compared to GFP controls was observed (FIG. 12B).

### Example 3

### Identification of Spi2A as a Protective Gene that Facilitates the Differentiation of Memory T Lymphocytes

### Materials and Methods

Mice. Wild type C57BL/6, RAG1^{-/-} C57BL/6 (Nombaerts et al., 1992), CD8^{-/-} C57BL/6 mice (Fueng-Leung et al., 1991) (obtained from The Jackson Laboratory), RAG1^{+/+} B6.2.16 (Kisielow et al., 1988) and RAG1^{-/-} B6.2.16 transgenic mice (Opferman et al., 1999) (129/SvJ×C57BL/6) were maintained and bred under standard specific pathogen free (SPF) conditions.

Anti-HY memory CD8 cells. Naïve B6.2.16 CD8 cells (>85% pure) were isolated from the lymph nodes (LN) of female RAG1-deficient B6.2.16 mice (Opferman et al., 1999). Anti-HY effectors were generated by culturing splenocytes from female RAG1-deficient B6.2.16 mice with HY peptide for 4 d as previously described (Markiewicz et al., 1998). Anti-HY effectors (>95% pure) were adoptively transferred into female RAG1-deficient mice. After 200 d, memory B6.2.16 CD8 cells (>80% pure) were recovered from the spleens and LNs by magnetic bead sorting with anti-thy1.2 beads (Miltenyi Biotec).

LCMV Infections. LCMV Armstrong was stored as high titer stocks as described before (Lin and Welsh, 1998). For primary infections C57BL/6 mice were infected by intra-peritoneal (i.p) injection of 2×10⁵ plaque forming units (PFU) of LCMV and for secondary infections 10⁶ PFU i.p. To obtain primary CTL effectors from the spleen, mice were sacrificed after 8 d and to obtain memory CD8 cells, mice were sacrificed no sooner than 80 days after infection. Secondary effectors were obtained from the spleen 5 d after re-infection of C57BL/6 mice, which were previously with LCMV 60 d before.

Flow Cytometric Analyses. The following mAbs were used: anti-CD8α (allophycocyanin [APC]-labeled), anti-B220 (R-phycoerythrin [PE] labeled), anti-CD44-PE, anti-IFN-γ-PE (rat IgG₁) and rat IgG₁-PE isotype control (Pharmingen). H-2D^{b}-tetramers were refolded with the following LCMV peptides: NP 396 [FQPQNGQFI (SEQ ID NO:23)], GP 33 [KAVYNFATM (SEQ ID NO:24)] or GP 276 [SGVENPGGYCL (SEQ ID NO:25)] and labeled with streptavidin-PE, as described previously (Ober et al., 2000). Suspensions of splenocytes were prepared after red blood cell lysis and Ficoll-purification (Coligan et al., 1995) and stained with a cocktail including all three PE-labeled tetramers (each at 5 μg/ml) and anti-CD8α mAb or a combination of other mAbs for 30 min at 4° C. in staining buffer as described before (Murali-Krishna et al., 1998). T cells were enriched by magnetic sorting with anti-thy1.2 beads before purification by FACS (MoFlo; DakoCytomation). Naïve cells (CD44^{low}CD8⁺) were FACS-purified from the spleens of un-infected C57BL/6 mice by staining with anti-CD8α-APC and anti-CD44-PE antibodies.

To detect functional memory cells, splenocytes (5×10⁶/ml in 0.2 ml) were incubated with all three LCMV peptide antigens (each at 10⁻⁷ M) for 5 h in the presence of Golgi-block according to manufacturer's instructions (Pharmingen). Cells were fixed in 1% paraformaldehyde, permeabilized with 0.3% saponin and stained with anti-IFN-γ or isotype control (rat IgG₁) mAb according to manufacturer's instructions.

Analysis of gene expression. RNA from B6.2.16 CD8 cells was isolated using Trizol® Reagent (Invitrogen) and used to make cRNA for hybridization with Affymetrix Gene Arrays® (Mu11KA and Mu11KB) according to the company's instructions. RNA from anti-LCMV CD8 cells was purified using Trizol® Reagent (Invitrogen), and then cDNA was generated using Superscript™ First-Strand Synthesis System for RT-PCR (Invitrogen). The unique specificity of each set of primers and probes was verified by checking the sequences against the GenBank database (which may be found at the National Institutes of Health website on the internet). Probes contained the fluorescent reporter dye FAM and either TAMRA or QSY7 as the quencher (MegaBases, Inc.). Real-time PCR reactions were carried out using TaqMan® Universal PCR Master Mix (PE Applied Biosystems) and run on an ABI Prism 7700 Sequence Detection System. The slope of the standard curve describes the efficiency of the real-time PCR, which allowed us to ensure that the real-time PCR reactions consistently ran at >90% efficiency. The relative RNA concentrations were calculated by dividing the concentration of candidate gene RNA by the concentration of the cyclophilin A control gene (Medhurst et al., 2000).

Retrovirally transduced bone-marrow chimeras. Donor C57BL/6 mice (8-10 w) were injected i.p. with 5-fluorouracil (150 mg/Kg; Sigma) and after 5 d bone marrow was harvested and plated in 24-well plates (10⁶/well) for 48 h in conditioned medium [DMEM with 15% heat-inactivated fetal calf serum, penicillin (10 U/ml), streptomycin (10 μg/ml), L-glutamine (2 mM), and β-mercaptoethanol (5×10⁻⁵ M), recombinant (r)-mouse IL-3 (20 ng/ml, Biosource International), IL-6 (10 ng/ml, R&D), r-mouse stem cell factor (50 ng/ml, Biosource International) and r-human flt3 ligand (50-100 ng/ml, R&D)]. Stem cells were then harvested and transduced with MIGR1 MuMLV (Zang and Ren, 1998). The production of MIGR1 empty control virus (GFP) or those viruses encoding Spi2A in the sense (Spi2A) or anti-sense orientation (Spi2A-A) on polycistronic messages with GFP has been described before (Liu et al., 2003). For transduction, bone-marrow stem cells (1.5×10⁶) were re-suspended in 24-well plates with retrovirus supernatants (2 ml/well) in conditioned medium containing polybrene (8 μg/ml) and centrifuged (1000×g) for 3 h at 4° C. After two days, C57BL/6 CD8-deficient mice (6-8 w) were γ-irradiated (1200 rads) then injected intravenously (i.v.) with transduced bone marrow (1.5−2.0×10⁶ cells/mouse).

Results

Identification of genes up-regulated in memory CD8 cells. Gene-array technology was used to broadly survey differences in gene expression between memory and naïve CD8 cells. In this study, CD8 cells that express the B6.2.16 transgenic TCR (Kisielow et al., 1988) that recognizes the male-specific HY peptide presented by H-2D^{b} (Markiewicz et al., 1998) were used. Naïve anti-HY CD8 cells were obtained directly from the lymph nodes of female RAG1-deficient B6.2.16 mice (Opferman et al., 1999). Anti-HY CTLs were generated by in vitro culture of B6.2.16 CD8 cells with HY peptide, and then adoptively transferred to antigen-free, RAG1-deficient mice. After 200 days, memory B6.2.16 CD8 cells were purified from the spleens of these recipients, as described previously (Markiewicz et al., 1998; Opferman et al., 1999). Analysis of approximately 11,000 mouse genes using RNA isolated from naïve and memory B6.2.16 CD8 cells revealed that 241 genes were significantly up-regulated by at least 2-fold in memory CD8 cells (Table 2 and Table 3).

TABLE 2
DNA 11KA ARRAY ANALYSIS OF GENE EXPRESSION IN B6.2.16
CD8 CELLS

| Accession | | Fold | Sort |
|---|---|---|---|
| Number | Gene | Change | Score |
| AA165775_s_at | EST | 42.5 | 20.58 |
| L28117_s_at | Mouse NF-kappa-B (p105) mRNA | 36.8 | 16.82 |
| aa199380_s_at | EST | 32.4 | 13.03 |
| aa415898_s_at | EST | 26.9 | 13.21 |
| aa617493_s_at | EST | 23.8 | 11.87 |
| aa542220_s_at | EST | 21.2 | 10.37 |
| aa616578_s_at | EST | 20.3 | 8.97 |
| u29947_s_at | Mus musculus alpha-D-mannosidase (Man2b1) mRNA, | 19.3 | 9.46 |
| aa178252_s_at | EST | 16.8 | 3.1 |
| J05261_f_at | Mouse protective protein (Mo54) mRNA, complete cds | 16.4 | 9.43 |
| m19681_s_at | Platelet-derived growth factor-inducible protein (JE) gene | 15.4 | 6.16 |
| aa521734_g_at | EST | 14.7 | 6.28 |
| aa711915_at | EST | 14.4 | 6.79 |
| m74294_s_at | Mouse IL-1m antagonist protein mRNA, complete cds | 14.4 | 6.47 |
| aa103548_s_at | EST | 14.2 | 4.88 |
| aa050541_s_at | EST | 13.8 | 4.96 |
| aa474495_at | EST | 12.2 | 0.8 |
| U59807_f_at | Mus musculus cystatin B (Stfb) gene, complete cds | 11.4 | 4.53 |
| aa546670_s_at | EST | 11.4 | 4.52 |
| U43085_s_at | Glucocorticoid-attenuated response gene 39 (GARG-39) mRNA | 10.8 | 4.8 |
| aa616057_at | EST | 10.8 | 3.8 |
| aa575696_at | EST | 10.5 | 4.59 |
| U51014_s_at | Mus musculus prolidase (pep4) mRNA, complete cds | 9.7 | 0.82 |
| aa184455_at | EST | 9.6 | 3.15 |
| aa124813_s_at | EST | 9.5 | 3.39 |
| aa616705_s_at | EST | 9.3 | 5.3 |
| AA403550_s_at | EST | 9.2 | 3.72 |
| aa050066_s_at | EST | 9 | 3.58 |
| I02333_s_at | Bilirubin/phenol family UDP glucuronosyltransferase (ugtBr) | 8.6 | 3.19 |
| I32974_s_at | Mouse interferon-inducible protein homologue mRNA | 8.5 | 7.73 |
| D18303_rc_at | Mouse 3′-directed cDNA; MUSGS01123; clone md1345 | 8.3 | 3.14 |
| I75822_s_at | Mus musculus follistatin-like protein mRNA, complete cds | 8.3 | 0.55 |
| X00686_3_at | X00686 Mouse gene for 18S rRNA | 8.2 | 4.83 |
| D44464_s_at | Mouse mRNA for uridine phosphorylase, complete cds | 8.2 | 2.07 |
| aa198402_s_at | EST | 8.2 | 1.55 |
| aa122717_at | EST | 8.1 | 2.86 |
| AA124895_s_at | EST | 8 | 2.78 |
| AA288280_s_at | EST | 7.9 | 2.8 |
| aa472322_at | EST | 7.9 | 2.77 |
| L32973_s_at | Mouse thymidylate kinase homologue mRNA, complete cds | 7.8 | 5.89 |
| U72643_s_at | lymphocyte specific transcript (LST) mRNA, partial cds | 7.2 | 8.47 |
| U18424_s_at | Mus musculus bacteria binding macrophage receptor MARCO | 7.2 | 2.03 |
| AA208776_s_at | EST | 7 | 2.7 |
| aa260827_s_at | EST | 7 | 1.42 |
| aa277739_at | EST | 6.9 | 1.27 |
| aa175784_s_at | EST | 6.8 | 9.08 |
| AA617408_rc_s | EST | 6.6 | 1.72 |
| U86137_s_at | Mus musculus telomerase protein-1 mRNA, complete cds | 6.6 | 1.7 |
| aa606536_s_at | EST | 6.6 | 1.58 |
| aa268226_g_at | EST | 6.4 | 2.16 |
| aa673555_at | EST | 6.2 | 2.37 |
| aa217241_s_at | EST | 6.2 | 1.92 |
| C78062_rc_f_at | EST | 6.2 | 1.89 |
| c75942_rc_s_at | EST | 6.2 | 1.63 |
| M12481_3_st | M12481 Mouse cytoplasmic beta-actin mRNA | 6.1 | 3.08 |
| AF013114_s_at | Cytokine receptor-like molecule (EBI3) mRNA, complete cds | 6.1 | 1.91 |
| aa270965_s_at | EST | 6.1 | 0.55 |
| aa137620_s_at | EST | 6 | 0.84 |
| aa620113_s_at | EST | 6 | 0.38 |
| aa116735_s_at | EST | 5.9 | 2.59 |
| aa462486_s_at | EST | 5.7 | 0.88 |
| aa153748_at | EST | 5.7 | 0.47 |
| k02782_s_at | complement component C3 mRNA, alpha and beta subunits | 5.6 | 2.87 |
| aa103570_s_at | EST | 5.6 | 1.37 |
| AB008895_g_at | Mus musculus mRNA for mGpi1p, complete cds. | 5.5 | 0.79 |
| aa285831_s_at | EST | 5.4 | 2.51 |
| U44731_s_at | Mus musculus putative purine nucleotide binding protein mRNA | 5.4 | 2.13 |
| j05663_s_at | Mouse vas deferens androgen related protein (MVDP) mRNA | 5.4 | 1.5 |
| u19482_s_at | Mus musculus C10-like chemokine mRNA, complete cds | 5.4 | 1.43 |
| aa543970_at | EST | 5.4 | 0.99 |
| I13732_s_at | Mouse macrophage-specific integral membrane protein (Nramp) | 5.1 | 3.16 |
| M55154_s_at | Mouse transglutaminase (TGase) mRNA, complete cds | 5.1 | 2.47 |
| af018268_at | Mus musculus Sp-alpha mRNA, complete cds. | 5.1 | 1.23 |
| L19932_s_at | Mouse (beta ig-h3) mRNA, complete cds | 5 | 4.56 |
| aa185385_s_at | EST | 5 | 1.38 |
| aa611449_at | EST | 5 | 1.25 |
| aa182189_at | EST | 4.9 | 5.48 |
| m31419_f_at | Mouse 204 interferon-activatable protein mRNA, complete cds | 4.9 | 2.36 |
| L42293_s_at | Mus musculus acyl-coenzyme A: cholesterol acyltransferase mR | 4.9 | 1.91 |
| u73004_s_at | Mus musculus secretory leukocyte protease inhibitor mRNA | 4.8 | 4.36 |
| d14566_f_at | Lmp-2 gene for LMP-2 polypeptide and Tap-1 gene (exon 1-3) | 4.8 | 2.28 |
| aa608160_s_at | EST | 4.8 | 0.95 |
| u09138_s_at | peroxisome proliferator activated protein-gamma-2 | 4.8 | 0.42 |
| M60474_f_at | Mouse myristoylated alanine-rich C-kinase substrate (MARCKS) | 4.7 | 3.8 |
| M58288_s_at | Mus musculus granulocyte colony-stimulating factor receptor | 4.7 | 2.85 |
| aa422356_s_at | EST | 4.7 | 1.74 |
| u05265_s_at | Mus musculus BALB/c gp49B gene | 4.6 | 3.72 |
| L38281_rc_g_at | immune-responsive gene 1 (Irg1) mRNA, 3′ end of cds | 4.6 | 2.46 |
| c77476_rc_at | EST | 4.6 | 2.01 |
| aa189512_g_at | EST | 4.5 | 2.66 |
| m12279_s_at | Interferon-induced Mx protein resistance to influenza virus | 4.5 | 2.05 |
| X00686_5_at | Mouse gene for 18S rRNA | 4.5 | 0.96 |
| aa266888_s_at | EST | 4.5 | 0.76 |
| C76739_rc_at | EST | 4.4 | 1.46 |
| d83648_s_at | Musculus domesticus mRNA for C-C chemokine receptor 5 | 4.4 | 1.04 |
| aa002704_at | EST | 4.4 | 0.7 |
| U19119_s_at | Mus musculus G-protein-like LRG-47 mRNA, complete cds | 4.3 | 2.15 |
| u06948_s_at | Mus musculus strain Balb/c Fas ligand mRNA | 4.3 | 1.64 |
| U70538_s_at | UDP-GalNAc:polypeptide N-acetylgalactosaminyltransferase-T3 | 4.3 | 0.98 |
| AA189702_at | EST | 4.2 | 4.15 |
| aa273574_f_at | EST | 4.2 | 2.81 |
| u05809_s_at | Mus musculus LAF1 transketolase mRNA, complete cds | 4.2 | 2.78 |
| U28168_s_at | EST | 4.2 | 2.73 |
| aa277088_s_at | EST | 4.2 | 1.86 |
| aa617442_s_at | EST | 4.2 | 0.77 |
| U76832_s_at | Mus musculus plasma membrane protein syntaxin-4 mRNA | 4.2 | 0.52 |
| af018268_g_at | Mus musculus Sp-alpha mRNA, complete cds. | 4.1 | 4.12 |
| aa212898_s_at | EST | 4.1 | 1.28 |
| aa182980_s_at | EST | 4.1 | 0.23 |
| aa543783_s_at | EST | 3.9 | 1.1 |
| aa230831_s_at | EST | 3.9 | 0.76 |
| aa682037_rc_at | EST | 3.9 | 0.63 |
| aa555736_s_at | EST | 3.9 | 0.31 |
| aa212981_s_at | EST | 3.8 | 2.92 |
| c81475_rc_f_at | EST | 3.8 | 1.53 |
| aa059717_s_at | EST | 3.8 | 1.39 |
| aa273845_s_at | EST | 3.8 | 1.39 |
| aa254525_i_at | EST | 3.8 | 0.76 |
| c77389_rc_s_at | EST | 3.8 | 0.34 |
| m33863_s_at | Mouse 2′-5′ oligo A synthetase mRNA, complete cds. | 3.7 | 3.36 |
| M93275_s_at | Mouse adipose differentiation related protein (ADFP) mRNA | 3.7 | 2.39 |
| aa185911_s_at | EST | 3.7 | 1.93 |
| aa014427_s_at | EST | 3.7 | 1.84 |
| m85153_s_at | M. musculus alpha-1,3-galactosyltransferase mRNA | 3.7 | 0.66 |
| aa267296_s_at | EST | 3.7 | 0.55 |
| k02109_f_at | Mouse 3T3-L1 lipid binding protein mRNA, complete cds | 3.7 | 0.42 |
| aa261246_s_at | EST | 3.6 | 3.25 |
| j03023_s_at | Murine macrophage gene, encoding bmk (B cell/myeloid kinase) | 3.6 | 2.65 |
| d37873_s_at | Mouse fcrn gene for Fc receptor | 3.6 | 1.81 |
| aa289002_s_at | EST | 3.6 | 1.67 |
| L07924_s_at | Guanine nucleotide dissociation stimulator for a ras-related | 3.6 | 1.62 |
| | GTPase | | |
| aa277082_g_at | EST | 3.6 | 1.61 |
| aa420407_rc_s | EST | 3.6 | 1.13 |
| u54984_s_at | Mus musculus membrane-type matrix metalloproteinase 1 mRNA | 3.6 | 0.74 |
| d13003_s_at | Mus musculus reticulocalbin mRNA, complete cds | 3.6 | 0.68 |
| c76527_rc_g_at | EST | 3.5 | 2.72 |
| U39827_s_at | Mus musculus putative G protein-coupled receptor TDAG8 | 3.5 | 1.22 |
| | (TDAG8) | | |
| j04953_f_at | Mouse gelsolin gene, complete cds | 3.5 | 0.48 |
| aa562600_s_at | EST | 3.5 | 0.19 |
| U73037_s_at | Mus musculus interferon regulatory factor 7 (mirf7) mRNA | 3.4 | 2.64 |
| m86736_f_at | Mouse acrogranin mRNA, complete cds | 3.4 | 2.23 |
| U56773_s_at | Mus musculus pelle-like protein kinase mRNA, complete cds | 3.4 | 1.27 |
| m89641_s_at | Mus musculus interferon alpha/beta receptor (IFNAR) mRNA | 3.4 | 0.85 |
| af004666_s_at | Mus musculus sodium-calcium exchanger (NCX1) mRN | 3.4 | 0.7 |
| U72941_s_at | Mus musculus annexin IV mRNA, complete cds. | 3.4 | 0.66 |
| aa274431_s_at | EST | 3.3 | 2.01 |
| m15131_s_at | Mouse interleukin 1-beta (IL-1-beta) mRNA, complete cds | 3.3 | 1.84 |
| aa684476_s_at | EST | 3.3 | 1.73 |
| aa009160_s_at | EST | 3.3 | 1.18 |
| aa271265_s_at | EST | 3.2 | 2.56 |
| aa473331_s_at | EST | 3.2 | 1.67 |
| aa288442_s_at | EST | 3.2 | 1.27 |
| aa104485_s_at | EST | 3.2 | 0.84 |
| d49956_s_at | Mouse mRNA for 8-oxo-dGTPase | 3.2 | 0.18 |
| d87967_s_at | Mouse mRNA for SHPS-1, complete cds | 3.1 | 2.2 |
| m59470_f_at | Mouse cystatin C mRNA, complete cds | 3.1 | 1.92 |
| m27960_s_at | Mouse interleukin-4 receptor (secreted form) mRNA, complete cds | 3.1 | 1.58 |
| m32370_s_at | Mouse transcription factor PU.1 mRNA, complete cds | 3.1 | 1.28 |
| AA61113_at | EST | 3.1 | 0.72 |
| j05287_s_at | Mouse lysosomal membrane glycoprotein (LAMP-2) | 3.1 | 0.7 |
| aa529056_g_at | EST | 3.1 | 0.49 |
| I03799_s_at | Mouse interleukin-1 beta convertase (IL-1bc) mRNA, complete | 3.1 | 0.47 |
| | cds | | |
| aa154376_s_at | EST | 3.1 | 0.31 |
| aa409826_rc_s | EST | 3 | 2.69 |
| AF002718_s_at | Mus musculus ATPase inhibitor (IF1) mRNA, complete cds | 3 | 1.09 |
| aa682062_rc_s | EST | 3 | 0.82 |
| AA267281_i_at | EST | 3 | 0.55 |
| AA183138_f_at | EST | 3 | 0.37 |
| m18466_f_at | Mouse lymphocyte differentiation antigen Ly-6C.2 mRNA | 2.9 | 2.41 |
| aa109873_s_at | mm02f05.r1 Mus musculus cDNA, 5′ end | 2.9 | 1.79 |
| aa266897_at | EST | 2.9 | 1.21 |
| aa286391_s_at | EST | 2.9 | 1.04 |
| aa199273_s_at | EST | 2.9 | 0.89 |
| M29855_s_at | Mouse interleukin-3 receptor mRNA, complete cds | 2.9 | 0.47 |
| aa407794_rc_at | EST | 2.9 | 0.45 |
| aa245242_s_at | EST | 2.8 | 1.96 |
| U15635_s_at | Mus musculus IFN-gamma induced (Mg11) mRNA, complete cds | 2.8 | 1.82 |
| aa217659_s_at | EST | 2.8 | 1.36 |
| aa407697_rc_s | EST | 2.8 | 1.07 |
| aa239477_s_at | EST | 2.8 | 1.04 |
| M34141_s_at | Mouse prostaglandin endoperoxide (PGG/H) mRNA, complete | 2.8 | 0.91 |
| | cds | | |
| L16462_s_at | Mus musculus hemopoietic-specific early response protein (A1) | 2.8 | 0.89 |
| I26489_s_at | Mus musculus furin (FUR) mRNA, complete cds | 2.8 | 0.86 |
| AF012129_at | Mus musculus putative DNA methyltransferase (Dnmt2) mRNA | 2.8 | 0.57 |
| m12302_s_at | Mouse C11 mRNA encoding T-cell specific protein CCPI | 2.8 | 0.51 |
| M73329_s_at | Mouse phospholipase C-alpha (PLC-alpha) mRNA, complete cds | 2.7 | 1.9 |
| aa185574_s_at | EST | 2.7 | 1.69 |
| aa170668_s_at | EST | 2.7 | 1.42 |
| c76527_rc_at | EST | 2.7 | 1.14 |
| M25825_f_at | Mouse tctex-1 mRNA, complete cds | 2.7 | 1.13 |
| M11024_at | Endogenous mammary tumor virus RNA, env gene and right LTR | 2.7 | 0.86 |
| M31314_s_at | Mouse high affinity IgG receptor (Fc-gamma RI) mRNA | 2.7 | 0.52 |
| u70475_s_at | p45 NF-E2 related factor 2 (NRF2) gene, exon 2 to exon 5 | 2.7 | 0.52 |
| AA169001_at | EST | 2.7 | 0.44 |
| D50264_s_at | Mouse mRNA for phosphatidylinositol glycan class F, complete | 2.7 | 0.23 |
| | cds. | | |
| AA231005_f_at | EST | 2.7 | 0.15 |
| L02241_s_at | Mouse protein kinase inhibitor (testicular isoform) mRNA | 2.7 | 0.14 |
| ab004664_g_at | Mus musculus DNA for Rab33B, exon 2 and complete cds | 2.7 | 0.13 |
| m37761_s_at | Mouse calcyclin mRNA, complete cds | 2.6 | 1.9 |
| AA178227_at | EST | 2.6 | 1.32 |
| aa210359_s_at | EST | 2.6 | 1.18 |
| aa000380_s_at | EST | 2.6 | 1.08 |
| aa285530_s_at | EST | 2.6 | 1.06 |
| u05837_s_at | Mus musculus B6/CBA beta-hexosaminidase (Hexa) mRN | 2.6 | 0.96 |
| aa691772_at | EST | 2.6 | 0.92 |
| U20159_s_at | Mus musculus 76 kDa tyrosine phosphoprotein SLP-76 | 2.6 | 0.86 |
| aa733351_s_at | EST | 2.6 | 0.85 |
| U71205_s_at | Mus musculus rit mRNA, complete cds | 2.6 | 0.77 |
| C77861_rc_s_at | EST | 2.6 | 0.73 |
| U88908_s_at | Mus musculus inhibitor of apoptosis protein 1 mRNA, complete | 2.6 | 0.64 |
| | cds. | | |
| aa289572_s_at | EST | 2.6 | 0.59 |
| c81612_rc_at | EST | 2.6 | 0.32 |
| U44940_s_at | Mus musculus quaking type I (QKI) mRNA, complete cds | 2.6 | 0.25 |
| aa105104_at | EST | 2.6 | 0.16 |
| aa386453_at | EST | 2.6 | 0.11 |
| aa162557_s_at | EST | 2.5 | 1.5 |
| m17440_s_at | Mouse MHC (H-2) S region complement component C4 gene | 2.5 | 1.19 |
| m94584_s_at | Mus musculus secretory protein (YM-1) mRNA | 2.5 | 1.12 |
| aa174394_f_at | EST | 2.5 | 0.94 |
| C79010_rc_at | EST | 2.5 | 0.92 |
| U06119_s_at | Mus musculus cathepsin H prepropeptide (ctsH) mRNA | 2.5 | 0.91 |
| aa408789_rc_s | EST | 2.5 | 0.8 |
| aa185007_s_at | EST | 2.5 | 0.76 |
| aa546047_s_at | EST | 2.5 | 0.76 |
| aa273938_s_at | EST | 2.5 | 0.43 |
| aa615853_s_at | EST | 2.5 | 0.4 |
| aa066610_g_at | EST | 2.5 | 0.36 |
| aa692678_i_at | EST | 2.5 | 0.25 |
| M74495_s_at | Mouse adenylosuccinate synthetase mRNA, complete cds | 2.5 | 0.25 |
| u56920_s_at | Mus musculus steroid receptor coactivator 1a (Src1a) mRNA | 2.5 | 0.17 |
| m22531_f_at | Mouse complement C1q B chain mRNA, complete cds | 2.4 | 1.56 |
| u24700_s_at | Mus musculus protein tyrosine phosphatase (HA2) mR | 2.4 | 1.25 |
| C79895_rc_at | EST | 2.4 | 1.19 |
| aa253918_at | EST | 2.4 | 0.96 |
| u07617_s_at | Mus musculus BALB/C Grb2 adaptor protein (grb2) mRNA | 2.4 | 0.96 |
| aa242556_s_at | EST | 2.4 | 0.95 |
| aa216920_s_at | EST | 2.4 | 0.81 |
| M32010_s_at | Mouse MHC H-2K/t-w5-linked open reading frame mRNA | 2.4 | 0.8 |
| AA266395_s_at | EST | 2.4 | 0.74 |
| C78749_rc_g | EST | 2.4 | 0.7 |
| U53219_s_at | Mus musculus GTPase IGTP mRNA, complete cds. | 2.4 | 0.68 |
| AF013099_at | Mus musculus multiubiquitin-chain-binding protein (Mcb1) mRNA | 2.4 | 0.65 |
| aa543785_g_at | EST | 2.4 | 0.56 |
| aa538478_s_at | EST | 2.4 | 0.55 |
| aa638884_s_at | EST | 2.4 | 0.51 |
| AA615066_g_at | EST | 2.4 | 0.41 |
| aa242340_s_at | EST | 2.4 | 0.23 |
| m64291_s_at | Mus musculus prostaglandin synthase mRNA, complete cds. | 2.4 | 0.15 |
| aa638539_s_at | EST | 2.3 | 1.06 |
| aa172851_s_at | EST | 2.3 | 0.93 |
| M65027_s_at | Mouse cell surface antigen gp49 mRNA, complete cds | 2.3 | 0.9 |
| U44426_s_at | Mus musculus D52 (mD52) mRNA, complete cds | 2.3 | 0.77 |
| aa711028_s_at | EST | 2.3 | 0.66 |
| aa403731_f_at | EST | 2.3 | 0.58 |
| L36314_f_at | Mus musculus GDP dissociation inhibitor beta mRNA, | 2.3 | 0.52 |
| D16432_s_at | Mouse murine CD63 mRNA for murine homologue of | 2.3 | 0.46 |
| | CD63/ME491 | | |
| M12481_M_st | M12481 Mouse cytoplasmic beta-actin mRNA | 2.3 | 0.45 |
| AA184228_s_at | EST | 2.3 | 0.34 |
| aa268084_s_at | EST | 2.3 | 0.33 |
| aa529389_s_at | EST | 2.3 | 0.19 |
| I02210_s_at | Mus musculus tyrosine kinase-related protein mRNA, complete | 2.3 | 0.09 |
| | cds | | |
| K01925_f_at | Mouse MHC class II H2-IA-alpha gene (q haplotype), mRNA | 2.2 | 0.92 |
| m57696_f_at | Mouse lyn A protein tyrosine kinase (lynA) mRNA, complete cds | 2.2 | 0.77 |
| D49949_s_at | Mouse mRNA for IGIF precursor polypeptide, complete cds | 2.2 | 0.71 |
| aa271499_s_at | EST | 2.2 | 0.7 |
| L12120_s_at | Mouse interleukin-10 receptor (Il10r) mRNA, complete cds | 2.2 | 0.64 |
| aa672840_s_at | EST | 2.2 | 0.61 |
| d85561_s_at | Mus musculus mRNA for proteasome subunit MECL1, complete | 2.2 | 0.6 |
| | cds. | | |
| aa198790_s_at | EST | 2.2 | 0.56 |
| a711151_s_at | EST | 2.2 | 0.51 |
| u57325_s_at | Mus musculus PS-2short mRNA, partial cds | 2.2 | 0.48 |
| aa276368_s_at | EST | 2.2 | 0.47 |
| AA407689_rc_s | EST | 2.2 | 0.42 |
| U44389_s_at | EST | 2.2 | 0.42 |
| D78135_s_at | Mouse mRNA for glycine-rich RNA binding protein CIRP | 2.2 | 0.39 |
| L36435_s_at | Mus musculus basic domain/leucine zipper transcription factor | 2.2 | 0.34 |
| aa275260_s_at | EST | 2.2 | 0.33 |
| C80444_rc_at | EST | 2.2 | 0.18 |
| af015284_s_at | Mus musculus selenoprotein W (mSelW) mRNA, complete cds. | 2.1 | 1.02 |
| aa119603_at | EST | 2.1 | 0.75 |
| m14215_s_at | Mouse Fc gamma receptor (IgG 2.4G2 receptor mRNA | 2.1 | 0.69 |
| I31532_g_at | Mus musculus bcl-2 alpha gene, exon 2 | 2.1 | 0.66 |
| U43673_s_at | Mus musculus putative transmembrane receptor IL-IRrp mRNA | 2.1 | 0.63 |
| aa182340_s_at | EST | 2.1 | 0.56 |
| aa611413_at | EST | 2.1 | 0.54 |
| aa274091_at | EST | 2.1 | 0.53 |
| d82019_f_at | Mouse gene for basigin precursor, basigin signal precursor | 2.1 | 0.5 |
| aa544884_s_at | EST | 2.1 | 0.44 |
| k01496_f_at | Mouse MHC, class III antigen, factor B (H-2d haplotype) | 2.1 | 0.43 |
| I25069_s_at | Mouse catalase mRNA, complete cds | 2.1 | 0.42 |
| AA183642_at | EST | 2.1 | 0.34 |
| U30838_s_at | EST | 2.1 | 0.33 |
| M63695_s_at | Mouse CD1.1 mRNA, complete cds | 2.1 | 0.32 |
| D18928_rc_at | EST | 2.1 | 0.15 |
| aa407332_at | EST | 2.1 | 0.07 |
| m11284_s_at | Mouse MHC class I Qa-Tla mRNA, (H2-d haplotype), clone pH2- | 2 | 0.89 |
| | d-37 | | |
| m25244_f_at | Mouse pre-B cell P2B/LAMP-1 mRNA, complete cds | 2 | 0.83 |
| u89269_s_at | Mus musculus preprodipeptidyl peptidase I mRNA, complete cds. | 2 | 0.78 |
| aa538556_g_at | EST | 2 | 0.74 |
| aa177433_s_at | EST | 2 | 0.73 |
| AF017630_at | Vascular actin single-stranded DNA-binding factor 2 p44 | 2 | 0.64 |
| aa726578_at | EST | 2 | 0.57 |
| m35244-2_s_at | Mouse MHC class I H2-TL-T10-129 mRNA (b haplotype) | 2 | 0.55 |
| ab009287_s_at | Mus musculus gene for Macrosialin, complete cds. | 2 | 0.54 |
| aa028770_i_at | EST | 2 | 0.52 |
| aa219775_s_at | EST | 2 | 0.51 |
| aa116686_s_at | House mouse; Musculus domesticus male brain mRNA for ARF1 | 2 | 0.48 |
| U58888_s_at | EST | 2 | 0.47 |
| M29697_s_at | Mouse interleukin-7 receptor (IL-7) mRNA, complete cds | 2 | 0.4 |
| D50494_s_at | Mouse mRNA for murine RCK, complete cds | 2 | 0.39 |
| U80819_s_at | Mus musculus glutathione-S-transferase homolog mRNA | 2 | 0.33 |
| aa607833_at | EST | 2 | 0.33 |
| J03776_s_at | Mouse down regulatory protein (rpt-1r) of interleukin | 2 | 0.31 |
| aa606712_at | EST | 2 | 0.25 |
| u29396_s_at | Mus musculus annexin V (Anx5) mRNA, complete cds | 2 | 0.24 |
| aa475660_s_at | EST | 2 | 0.24 |
| aa543494_s_at | EST | 2 | 0.23 |
| k00020_s_at | mouse interferon-beta mrna | 2 | 0.19 |
| AA409042_rc | EST | 2 | 0.06 |
| aa467619_s_at | EST | −2 | −0.33 |
| C79965_rc_i_at | EST | −2 | −0.33 |
| D38613_s_at | Mouse 921-L mRNA for presynaptic protein, complete cds | −2 | −0.43 |
| aa172909_f_at | EST | −2 | −0.96 |
| aa388848_s_at | EST | −2 | −1.07 |
| U70210_s_at | TR2L mRNA PIR: JC5060 (tumor necrosis factor resistant protein | −2.1 | −0.28 |
| aa285607_s_at | EST | −2.1 | −0.31 |
| aa199023_at | EST | −2.1 | −0.44 |
| C78859_rc_at | EST | −2.1 | −0.48 |
| U69535_s_at | Mus musculus semaphorin M-sema G mRNA | −2.1 | −0.49 |
| u23462_s_at | Mus musculus CD7 antigen (Cd7) gene, complete cds. | −2.1 | −0.64 |
| L29441_f_at | Mus musculus mRNA, complete cds | −2.1 | −0.65 |
| c80656_rc_at | EST | −2.1 | −1.33 |
| aa543724_f_at | EST | −2.1 | −1.56 |
| M83219_s_at | Mus musculus intracellular calcium-binding protein (MRP14) | −2.1 | −1.57 |
| | mRNA | | |
| I37297_s_at | Mus musculus (clone B6) myeloid secondary granule protein | −2.1 | −1.61 |
| | mRNA | | |
| AA230776_f_at | EST | −2.1 | −1.87 |
| D17577_s_at | Mouse mRNA for kinesin-like protein (Kif1b), complete cds | −2.2 | −0.25 |
| AA617397_rc_s | EST | −2.2 | −0.78 |
| M21065_s_at | Mouse interferon regulatory factor 1 mRNA, complete cds | −2.2 | −1.4 |
| AA432883_f_at | EST | −2.2 | −2.17 |
| m83218_s_at | Mus musculus intracellular calcium-binding protein (MRP8) mRNA | −2.2 | −2.18 |
| L31609_s_at | S29 ribosomal protein mRNA, complete cds | −2.2 | −2.44 |
| U21855_s_at | Mus musculus mCAF1 protein mRNA, complete cds | −2.3 | −0.12 |
| M68944_s_at | Mouse alpha-interferon gene, complete cds | −2.3 | −0.17 |
| aa546953_at | EST | −2.3 | −0.36 |
| aa021959_s_at | EST | −2.3 | −0.38 |
| u28807_s_at | lymphoid-specific transcription factor NFATc3 mRNA, partial cds | −2.3 | −0.6 |
| aa409750_at | EST | −2.3 | −0.87 |
| m14343_s_at | Ly-5 (leucocyte-common antigen) pseudogene mRNA, clone R2 | −2.3 | −1.58 |
| AA409561_rc_f | EST | −2.3 | −2 |
| C80754_rc_f_at | EST | −2.3 | −2.21 |
| aa518802_s_at | EST | −2.3 | −2.29 |
| AA709861_f_at | EST | −2.3 | −2.35 |
| U93863_s_at | Mus musculus ribosomal protein L21 mRNA | −2.3 | −2.63 |
| u10551_s_at | Mus musculus Gem GTPase (gem) mRNA | −2.4 | −0.47 |
| aa521747_s_at | EST | −2.4 | −0.57 |
| u20735_s_at | Mus musculus transcription factor junB (junB) gene, 5′ region | −2.4 | −1.56 |
| aa529064_f_at | EST | −2.4 | −2.05 |
| c76068_rc_g_at | EST | −2.4 | −3 |
| C78891_rc_at | EST | −2.5 | −0.17 |
| C78676_rc_f_at | EST | −2.5 | −1.81 |
| C79877_rc_f_at | EST | −2.5 | −1.97 |
| M29475_s_at | Mouse recombination activating protein (RAG-1) mRN | −2.6 | −0.19 |
| AA124090_r_at | EST | −2.6 | −0.26 |
| m76763_f_at | Mus musculus ribosomal protein (Ke-3) mRNA, complete cds | −2.6 | −3.08 |
| U11274_s_at | Mus musculus clone pmuAUF1-3 RNA-binding protein AUF1 | −2.7 | −0.42 |
| | mRNA | | |
| u80036_s_at | Mus musculus orthodenticle-like homeobox 2 (Otlx2) | −2.7 | −0.55 |
| aa175340_s_at | EST | −2.7 | −0.64 |
| U11248_f_at | Mus musculus C57BL/6J ribosomal protein S28 mRNA | −2.7 | −3.45 |
| U49861_s_at | Mus musculus type 1 deiodinase (DIO1) mRNA | −2.8 | −0.28 |
| AF011424_s_at | Mus musculus putative pheromone receptor (VR14) mRNA | −2.8 | −0.56 |
| J00475_V0082 | Mouse germline IgH chain gene, DJC region: segment D-FL16.1 | −2.8 | −1.28 |
| d00208_s_at | pEL98 protein mRNA which is enhanced cells Balb/c373 | −2.8 | −1.74 |
| aa538407_s_at | EST | −2.8 | −3.21 |
| D18860_rc_at | EST | −2.9 | −0.61 |
| AA060336_at | EST | −2.9 | −0.99 |
| m12815_g_at | T-cell receptor active gamma-chain from cytotoxic T cell line | −2.9 | −1.3 |
| j04716_f_at | Mouse ferritin light chain, complete cds. | −2.9 | −3.92 |
| M12481_M_at | M12481 Mouse cytoplasmic beta-actin mRNA | −2.9 | −4.61 |
| c81467_rc_g_at | EST | −3 | −0.42 |
| AF031568_s_at | Mus musculus heterogeneous nuclear ribonucleoprotein G | −3 | −0.42 |
| | (Hnrnpg) | | |
| aa614984_f_at | EST | −3 | −2.74 |
| j04181_f_at | Mouse A-X actin mRNA, complete cds | −3 | −3.46 |
| D50527_f_at | Mouse mRNA for TI-225, complete cds. | −3 | −4.05 |
| M12481_5_at | M12481 Mouse cytoplasmic beta-actin mRNA | −3 | −4.41 |
| aa538404_g_at | EST | −3 | −5.06 |
| u52951_s_at | Mus musculus putative transcriptional regulator mEnx-1 mRNA | −3.1 | −0.68 |
| u58494_f_at | Intracisternal A-particle mRNA, gag gene, pol and env | −3.1 | −1.78 |
| | pseudogenes | | |
| aa036204_s_at | EST | −3.2 | −4.94 |
| U29402_f_at | Mus musculus acidic ribosomal phosphoprotein P1 mRNA | −3.2 | −5.11 |
| u47329_f_at | Mus musculus MHC class I heavy chain precursor (H-2K(d)) | −3.2 | −5.62 |
| | mRNA | | |
| aa170492_s_at | EST | −3.3 | −1.47 |
| M33330_f_at | Mouse insulinoma (rig) mRNA, complete cds | −3.3 | −4.6 |
| U93862_s_at | Mus musculus ribosomal protein L41 mRNA, complete cds | −3.4 | −6.34 |
| m22432_f_at | protein synthesis elongation factor Tu(eEF-Tu, eEf-1-alpha)mRNA | −3.4 | −6.47 |
| U25096_s_at | Mus musculus Kruppel-like factor LKLF mRNA, complete cds | −3.5 | −3.3 |
| AA691239_f_at | EST | −3.5 | −4.86 |
| C79965_rc_f_at | EST | −3.5 | −5.51 |
| AA673251_rc | EST | −3.8 | −1.06 |
| aa044510_s_at | EST | −3.9 | −1.82 |
| C79964_rc_f_at | EST | −3.9 | −5.49 |
| m16118_at | mRNA for T-cell receptor insulin (A-chain) reactive alpha chain | −4 | −7.9 |
| | VJC | | |
| AB000777_at | Mouse mRNA for photolyase/blue-light receptor homolog | −4.1 | −0.69 |
| c79329_rc_at | EST | −4.1 | −0.85 |
| U66575_rc_at | STAT6 (Stat6) gene, partial cds and NAB2 (Nab2) gene, 3′UTR | −4.1 | −1.46 |
| C76057_rc_f_at | EST | −4.1 | −7.06 |
| C77806_rc_f_at | EST | −4.2 | −1.31 |
| aa220788_s_at | EST | −4.3 | −0.25 |
| AA10605_f_at | EST | −4.3 | −7.36 |
| u04268_s_at | Mus musculus C57BL/6 Sca-2 precursor mRNA, complete cds | −4.3 | −7.69 |
| aa616325_g_at | EST | −4.4 | −1.1 |
| c76944_rc_f_at | EST | −4.5 | −10.6 |
| u78085_s_at | Mus musculus ribosomal protein S5 mRNA, complete cds | −4.6 | −10 |
| aa538062_at | EST | −4.7 | −1.17 |
| C79473_rc_f_at | EST | −4.8 | −11.8 |
| D84391_f_at | Mouse L1 repetitive element, complete sequence. | −4.9 | −6.65 |
| AA673251_rc_g | EST | −5.1 | −1.27 |
| aa118259_at | EST | −5.2 | −1.46 |
| aa190087_at | EST | −5.2 | −1.73 |
| M12481_3_at | M12481 Mouse cytoplasmic beta-actin mRNA | −5.4 | −13.6 |
| AA590859_f_at | EST | −5.5 | −13.2 |
| aa407468_rc_s | EST | −5.7 | −2.66 |
| AA675026_rc_f | EST | −5.7 | −4.28 |
| c75983_rc_f_at | EST | −5.7 | −9.52 |
| c76162_rc_f_at | EST | −6 | −8.17 |
| AA016609_at | EST | −6.1 | −1.9 |
| aa217487_s_at | EST | −6.2 | −6.19 |
| m25812_s_at | Mouse T lymphocyte antigen (A1) mRNA, complete cds | −6.3 | −2.12 |
| U12236_s_at | Mus musculus AKR alpha M290 integrin mRNA, complete cds | −6.3 | −3.76 |
| aa666971_f_at | EST | −7 | −19.7 |
| m54996_s_at | T-cell receptor gamma-chain mRNA, clone MNG8 | −7.7 | −3.92 |
| D19392_rc_f_at | EST | −8.7 | −7.6 |
| aa038322_s_at | EST | −10.6 | −5.18 |
| c79507_rc_f_at | EST | −11 | −19.9 |
| u28493_s_at | Mus musculus lymphotactin, exon 3 and complete cds | −11.8 | −5.13 |
| C79775_rc_f_at | EST | −12 | −27.7 |
| m12848_s_at | Mouse myb proto-oncogene mRNA encoding 71 kd myb protein | −13.7 | −7.74 |
| aa008853_s_at | EST | −22.1 | −10.7 |
| aa120608_s_at | EST | −23.5 | −11.7 |
| AA261028_f_at | EST | −32.4 | −32.4 |

TABLE 3
DNA 11KB ARRAY ANALYSIS OF GENE EXPRESSION IN B6.2.16
CD8 CELLS

| | | Fold | Sort |
|---|---|---|---|
| Accession Number | Gene | Change | Score |
| ET62206_r_at | Anti-digoxin immunoglobulin heavy chain variable region | 47.6 | 36.62 |
| Msa.4190.0_s_at | EST | 27.7 | 19.26 |
| Msa.717.0_s_at | Mouse glycerophosphate dehydrogenase gene, complete cds | 21.9 | 14.84 |
| Msa.3906.0_f_at | EST | 20 | 13.63 |
| Z31557_s_at | (129/Sv)Cctz mRNA for chaperonin containing TCP-1 | 19.2 | 14 |
| Msa.556.0_f_at | Mouse creatine kinase B gene, complete cds | 17.2 | 11.78 |
| x62742_s_at | M. musculus Ma mRNA. | 16 | 10.13 |
| Msa.1903.0_s_at | H2-M alpha chain (H2-Ma) gene, H2-M beta 2 chain | 10.8 | 6.13 |
| Msa.9251.0_s_at | EST | 10.7 | 6.17 |
| w58861_s_at | EST | 9.8 | 3.61 |
| x63027-2_at | M. musculus DNA for VSAG13 (viral superantigen) | 8 | 6.36 |
| Msa.6056.0_s_at | EST | 8 | 11.53 |
| Msa.12592.0_l_at | Homologous to sp P18085: ADP-RIBOSYLATION FACTOR 4 | 7.9 | 3.67 |
| x56602_s_at | Mus musculus mRNA Interferon-induced 15-KDa protein. | 7.9 | 12.27 |
| Msa.510.0_f_at | Mouse factor B mRNA, complete cds | 7.7 | 6.59 |
| X00686_5_at | Mouse gene for 18S rRNA | 7.5 | 3.89 |
| Msa.12676.0_at | Homologous to TRNA-PROCESSING PROTEIN SEN3 | 7.5 | 0.86 |
| Msa.3665.0_s_at | EST | 7.2 | 5.85 |
| Msa.16995.0_s_{—} | EST | 7.2 | 5.34 |
| Msa.853.0_f_at | EST | 6.7 | 5.24 |
| X61800_s_at | M. musculus mRNA for C/EBP delta | 6.6 | 2.33 |
| Z22661_f_at | M. musculus Apoc1 gene, exons 1 to 3 and complete CDS | 6.6 | 2.64 |
| X00686_3_at | Mouse gene for 18S rRNAns | 6.6 | 5.49 |
| Msa.4941.0_s_at | EST | 6.4 | 2.21 |
| Msa.2361.0_s_at | EST | 6.1 | 3.01 |
| w71676_at | EST | 6 | 5.18 |
| Msa.1700.0_s_at | Mus musculus PAF acetylhydrolase mRNA, complete cds | 5.7 | 7 |
| x01450_s_at | Mouse mRNA for interleukin-1. | 5.6 | 0.52 |
| x03479_s_at | Mouse mRNA fragment for serum amyloid A (SAA) 3 protein | 4.9 | 2.32 |
| Msa.1108.0_f_at | Mouse protective protein (Mo54) mRNA, complete cds | 4.8 | 4.14 |
| X54966_rc_at | M. musculus ctsB gene (3′ end) for cathepsin B. | 4.8 | 1.71 |
| Msa.5010.0_s_at | EST | 4.8 | 1.67 |
| w65767_s_at | EST | 4.8 | 1.59 |
| Msa.40135.0_s_{—} | EST | 4.6 | 1.87 |
| Msa.954.0_s_at | Mus musculus TAP2 (TAP2-d) mRNA, complete cds | 4.6 | 2.13 |
| ET62928_r_at | M. musculus antibody heavy chain variable region (366bp) | 4.6 | 1.35 |
| z12297_s_at | M. musculus mRNA for intercrine | 4.5 | 1.29 |
| Msa.23863.0_f_a | EST | 4.5 | 0.7 |
| Msa.16075.0_s_a | CREATINE KINASE, B CHAIN (EC 2.7.3.2) | 4.3 | 6.16 |
| Msa.1843.0_at | Macrophage inflammatory protein-1 alpha receptor | 4.2 | 1.44 |
| Msa.30006.0_s_a | EST | 4.2 | 0.89 |
| Msa.928.0_f_at | Myristoylated alanine-rich C-kinase substrate (MARCKS) | 4 | 4.12 |
| Msa.739.0_s_at | C57BL/6J ob/ob haptoglobin mRNA, complete cds | 3.9 | 3.86 |
| Msa.1867.0_s_at | Vacuolar adenosine triphosphatase subunit A gene | 3.8 | 0.31 |
| Msa.21652.0_f_a | AA027619 mi08d05.r1 Mus musculus cDNA, 5′ end | 3.8 | 3.9 |
| x06746_s_at | Mouse mRNA for Krox-20 protein containing zinc fingers | 3.8 | 0.82 |
| Msa.497.0_s_at | M. musculus mRNA for calcyclin | 3.8 | 1.7 |
| Msa.1600.0_s_at | Mus musculus MPS1 gene and mRNA, 3′end | 3.7 | 4.96 |
| w41301_s_at | EST | 3.7 | 0.61 |
| Msa.339.0_at | Mus musculus C57BL/6 platelet-type 12-lipoxygenase | 3.6 | 0.59 |
| Msa.22134.0_s_a | EST | 3.6 | 2.89 |
| Msa.10860.0_s_a | EST | 3.6 | 1.05 |
| Msa.918.0_f_at | Musculus domesticus mRNA for Ly-6C variant | 3.5 | 4.42 |
| Msa.5523.0_s_at | EST | 3.5 | 1.2 |
| X70100_f_at | M. musculus mal1 mRNA for keratinocyte lipid-binding protein | 3.4 | 2.29 |
| Msa.18459.0_f_a | EST | 3.4 | 3.36 |
| Msa.43194.0_f_a | M. musculus membrane glycoprotein gene | 3.3 | 1.57 |
| X16874_f_at | Mouse mRNA for complement protein C1q B-chain | 3.3 | 3.42 |
| Msa.1583.0_s_at | Mouse primary response gene B94 mRNA, 3′end | 3.3 | 2.65 |
| z49877_s_at | M. musculus syk mRNA for protein-tyrosine kinase. | 3.3 | 2.88 |
| ET61114_f_at | Musculus domesticus mRNA for Ly-6C variant | 3.2 | 3.82 |
| x04972_s_at | Mouse mRNA for manganese superoxide dismutase (MnSOD) | 3.2 | 2 |
| x67809_s_at | M. musculus mama mRNA. | 3.1 | 2.02 |
| Z11974_s_at | M. musculus mRNA for macrophage mannose receptor | 3.1 | 2.58 |
| Msa.8157.0_s_at | EST | 3.1 | 2.23 |
| X67644_s_at | M. musculus gly96 mRNA. | 3.1 | 2.13 |
| Msa.2433.0_f_at | EST | 3.1 | 0.91 |
| ET62839_at | Immunoglobulin-like receptor PIRA1 (6M21) mRNA | 3.1 | 1.2 |
| X61147_s_at | M. musculus mRNA for iron responsive element binding protein. | 3 | 1.06 |
| Msa.4559.0_s_at | EST | 3 | 1.02 |
| Msa.41726.0_f_a | AA163967 mr24g07.r1 Mus musculus cDNA, 5′ end | 3 | 0.5 |
| Msa.2623.0_g_at | M. musculus mRNA for GTP-binding protein | 2.9 | 0.89 |
| Msa.16148.0_s_a | EST | 2.8 | 1.45 |
| Msa.126.0_s_at | Mouse Bax alpha mRNA, complete cds | 2.8 | 0.93 |
| Msa.3356.0_s_at | EST | 2.8 | 0.76 |
| Msa.3138.0_s_at | EST | 2.7 | 0.98 |
| Msa.23293.0_f_a | AA048604 mj32g02.r1 Mus musculus cDNA, 5′ end | 2.7 | 1.54 |
| AFFX-b-ActinMur | Mouse cytoplasmic beta-actin mRNA | 2.7 | 1.15 |
| w91378_s_at | EST | 2.7 | 1.45 |
| Msa.30325.0_f_a | AA097051 mm51b03.r1 Mus musculus cDNA, 5′ end | 2.6 | 1.63 |
| w99005_s_at | EST | 2.6 | 0.69 |
| ET61664_s_at | Mus musculus FcgammaRIIB mRNA, complete cds. | 2.6 | 0.89 |
| Msa.43200.0_s_a | Mouse mRNA for transcription factor S-II-related protein | 2.6 | 0.17 |
| X58472_at | Mouse KIN17 mRNA for kin17 protein. | 2.6 | 0.31 |
| v01527_s_at | Major histocompatibility class II antigen, I-A-beta | 2.6 | 2.25 |
| w71236_s_at | Mus musculus FcgammaRIIB mRNA, complete cds. | 2.5 | 1.38 |
| Msa.41362.0_f_a | AA170251 ms87e11.r1 Mus musculus cDNA, 5′ end | 2.5 | 1.4 |
| Msa.17631.0_f_a | Mouse adenylosuccinate synthetase mRNA, complete cds | 2.5 | 0.91 |
| Msa.4956.0_f_at | EST | 2.5 | 0.81 |
| x56548_s_at | M. musculus Np-b mRNA for purine-nucleoside phosphorylase | 2.4 | 1.24 |
| Msa.3943.0_s_at | EST | 2.4 | 0.5 |
| Msa.1660.0_f_at | Mus musculus medium-chain acyl-CoA dehydrogenase | 2.4 | 0.63 |
| Msa.920.0_f_at | Mus musculus medium-chain acyl-CoA dehydrogenase | 2.4 | 1.38 |
| X06368_s_at | Murine mRNA for c-fms proto-oncogene. | 2.4 | 1.75 |
| w45964_s_at | EST | 2.4 | 1.03 |
| w12941_s_at | EST | 2.4 | 2.52 |
| X12616_s_at | Mouse c-fes proto-oncogene mRNA for c-fes protein | 2.4 | 1.07 |
| Msa.37791.0_s_a | EST | 2.4 | 0.55 |
| Msa.4469.0_at | EST | 2.4 | 0.62 |
| w08016_s_at | EST | 2.4 | 0.63 |
| Msa.31925.0_f_a | EST | 2.4 | 1.09 |
| Msa.477.0_f_at | Mouse mRNA for Mac-2 antigen | 2.4 | 1.77 |
| Msa.22422.0_s_a | EST | 2.3 | 1.09 |
| y00964_s_at | M musculus mRNA for beta-hexosaminidase | 2.3 | 1.01 |
| M12481_M_st | Mouse cytoplasmic beta-actin mRNA | 2.3 | 0.59 |
| x62743_s_at | M. musculus Mb mRNA | 2.3 | 1.29 |
| Msa.5619.0_s_at | EST | 2.3 | 1.04 |
| Msa.2614.0_at | Mouse mRNA for properdin (AA5-441) | 2.3 | 0.51 |
| Msa.2476.0_at | Murine MyD88 mRNA induced by interleukin-6 | 2.3 | 1 |
| Msa.38948.0_s_a | EST | 2.3 | 1.04 |
| Msa.5595.0_s_at | EST | 2.2 | 0.43 |
| x59379_s_at | mRNA for amyloid beta precursor (protease nexin II) | 2.2 | 1.09 |
| Msa.39606.0_s_a | mRNA for amyloid beta precursor (protease nexin II) | 2.2 | 0.6 |
| x04648_s_at | Mouse mRNA for IgG1/IgG2b Fc receptor (FcR) | 2.2 | 0.76 |
| x75129_s_at | (129/Sv) gene for xanthine dehydrogenase, exon 1 | 2.2 | 1.01 |
| y07711_s_at | M. musculus mRNA for zyxin | 2.2 | 1.04 |
| Msa.61.0_f_at | Mouse acrogranin mRNA | 2.2 | 1.35 |
| Msa.1178.0_s_at | Mouse spi2 proteinase inhibitor (spi2/eb1) mRNA, 3′ end | 2.2 | 1.61 |
| Msa.41995.0_s_a | EST | 2.2 | 0.82 |
| Msa.465.0_f_at | Mouse complement C1q B chain mRNA | 2.2 | 1.68 |
| Msa.25029.0_s_a | EST | 2.1 | 0.45 |
| Msa.547.0_f_at | Mouse mRNA for major excreted protein (MEP) | 2.1 | 0.32 |
| w82380_s_at | EST | 2.1 | 0.66 |
| x59769_s_at | Mouse II-1r2 mRNA for type II interleukin-1 receptor | 2.1 | 0.78 |
| Msa.1055.0_s_at | porphobilinogen deaminase (PBG deaminase) gene, exon 4 | 2.1 | 0.72 |
| z27231_s_at | M. musculus mRNA for type IV collagenase (gelatinase B) | 2.1 | 0.52 |
| y12650_s_at | Gene encoding hereditary haemochromatosis-like protein | 2.1 | 0.36 |
| Msa.5142.0_s_at | EST | 2.1 | 0.48 |
| w10325_s_at | EST | 2 | 0.78 |
| Msa.1023.0_s_at | EST | 2 | 0.27 |
| Msa.10058.0_s_a | EST | 2 | 1.22 |
| Msa.37429.0_s_a | EST | 2 | 0.53 |
| Z11911_s_at | mRNA for glucose-6-phosphate dehydrogenase | 2 | 1.11 |
| x60304_at | M. musculus mRNA for protein kinase C-delta | 2 | 0.9 |
| u96689_s_at | Immunoglobulin-like receptor PIRB1 (7M5) | 2 | 0.82 |
| Msa.463.0_at | Mus musculus bcl-2 alpha gene, exon 2 | 2 | 0.71 |
| X15591_s_at | Ctla-2-alpha homolog. to cysteine protease proregion | 2 | 1.18 |
| Msa.33501.0_f_a | EST | 2 | 0.65 |
| Z46757_s_at | M. musculus mRNA for high mobility group 2 protein | −2 | −0.58 |
| x03151_s_at | Mouse gene for Thy-1 antigen | −2 | −1.75 |
| Msa.17042.0_f_a | EST | −2 | −0.65 |
| X65627_f_at | M. musculus mRNA TNZ2 for p68 RNA helicase | −2 | −1.31 |
| L36135_i_at | Mouse mRNA for T-cell receptor delta-chain. | −2 | −0.58 |
| x14799_s_at | Autocrine thymic lymphoma cell mRNA for serine protease | −2 | −1.13 |
| Msa.26042.0_s_a | EST | −2 | −0.81 |
| x75313_f_at | M. musculus (C57BL/6) GB-like mRNA. | −2 | −1.75 |
| x03040_f_at | Mouse mRNA for initiation factor elF-4A long form | −2 | −1.26 |
| Msa.136.0_s_at | Common cytokine receptor gamma chain gene | −2.1 | −0.72 |
| Msa.1606.0_f_at | Mus musculus mRNA, complete cds | −2.1 | −1.2 |
| v00727_s_at | Mouse c-fos oncogene | −2.1 | −1.93 |
| x12592_s_at | Mouse mRNA of mkr3 gene encoding zinc finger protein | −2.1 | −0.46 |
| x81987_f_at | mRNA for TAX responsive element binding protein 107 | −2.2 | −2.19 |
| Msa.869.0_f_at | Mouse junD proto-oncogene mRNA, complete cds | −2.2 | −2.07 |
| X51829_s_at | Myeloid differentiation primary response mRNA | −2.2 | −1.35 |
| Msa.2707.0_f_at | EST | −2.2 | −2.37 |
| Msa.1734.0_f_at | EST | −2.2 | −2.37 |
| Msa.29510.0_f_a | EST | −2.2 | −0.73 |
| Msa.27798.0_s_a | EST | −2.2 | −0.16 |
| Z83368_f_at | M. musculus RPS3a gene | −2.3 | −2.87 |
| Msa.1076.0_at | Prim-1 proto-oncogene encoding pim-1 protein kinase | −2.3 | −2.59 |
| Msa.2195.0_f_at | Homology to yeast L29 ribosomal protein gene | −2.3 | −2.55 |
| x96606_s_at | M. musculus mRNA for Ott protein, clone ak27 | −2.3 | −0.13 |
| Msa.778.0_f_at | Mus musculus heat shock 70 protein (Hsc70) gene | −2.3 | −2.59 |
| X52803_f_at | Mouse mRNA for cyclophilin (EC 5.2.1.8) | −2.4 | −2.87 |
| Msa.6292.0_s_at | EST | −2.4 | −0.23 |
| Msa.10769.0_at | Homologous to RAS-RELATED PROTEIN RAB-7 | −2.4 | −0.32 |
| Msa.12956.0_s_a | EST | −2.4 | −0.7 |
| Msa.10932.0_s_a | Homologous to HEAT SHOCK PROTEIN HSP 90-ALPHA | −2.4 | −1.12 |
| Msa.8059.0_s_at | EST | −2.5 | −1.16 |
| Msa.28725.0_f_a | EST | −2.6 | −3.71 |
| Msa.43204.0_i_at | Mus musculus alpha-1 protease inhibitor 1 (alpha-1 PI-1) | −2.6 | −0.37 |
| Msa.1255.0_i_at | Thyrotropin beta-subunit (TSH-beta) gene, exons 3 and 4 | −2.6 | −0.39 |
| Msa.39913.0_f_{—} | EST | −2.7 | −0.82 |
| Msa.32149.0_f_a | EST | −2.7 | −3.76 |
| Msa.911.0_at | Mouse liver receptor homologous protein (LRH-1) mRNA | −2.7 | −0.61 |
| Msa.12361.0_s_a | W54839 ma28g09.r1 Mus musculus cDNA, 5′ end | −2.7 | −0.81 |
| Msa.1663.0_f_at | Mus musculus 5E6 (5E6/Ly-49C)) mRNA, complete cds | −2.7 | −0.76 |
| X05021_f_at | mRNA with homology to yeast 129 ribosomal protein gene | −2.7 | −3.87 |
| Msa.962.0_f_at | EST | −2.7 | −4.17 |
| Msa.1652.0_at | Mouse filaggrin mRNA, 3′ end | −2.7 | −0.6 |
| ET61037_f_at | Mouse mRNA for TI-225, complete cds | −2.8 | −4.97 |
| x60289_f_at | M. musculus mRNA for ribosomal protein S24. | −2.8 | −5.16 |
| Msa.2162.0_f_at | Mouse mRNA for acidic ribosomal phosophoprotein PO | −2.8 | −4.7 |
| Msa.24085.0_f_a | EST | −2.8 | −4.43 |
| Msa.17336.0_f_a | EST | −2.8 | −4.02 |
| Msa.22727.0_f_a | EST | −2.8 | −3.08 |
| W08025_at | EST | −2.8 | −0.26 |
| z32815_s_at | M. musculus net mRNA. | −2.9 | −1.16 |
| Msa.29918.0_f_a | EST | −2.9 | −4.35 |
| X15267_f_at | Mouse mRNA for acidic ribosomal phosophoprotein PO | −2.9 | −4.94 |
| X52634_f_at | Murine tlm oncogene for tlm protein | −3 | −5.05 |
| ET62689_at | Mus musculus T cell receptor V-beta 8 mRNA, partial cds | −3.1 | −0.87 |
| Msa.427.0_f_at | Amy-2.1 gene encoding amylase isozyme A-1 exon 5 | −3.1 | −0.59 |
| X15962_f_at | Mouse mRNA for ribosomal protein S12 | −3.1 | −6.71 |
| Msa.2412.0_f_at | CALCIUM-TRANSPORTING ATPASE PLASMA MEMBRANE | −3.1 | −7.02 |
| x14897_s_at | Mouse fosB mRNA | −3.1 | −4.49 |
| X00496_s_at | Mouse Ia-associated invariant chain (li) mRNA fragment | −3.1 | −5.75 |
| Msa.428.0_f_at | mouse alpha-amylase-like gene, partial | −3.2 | −0.57 |
| Msa.17018.0_f_a | W83919 mf33f11.r1 Mus musculus cDNA, 5′ end | −3.2 | −8.27 |
| X73331_f_at | M. musculus mRNA for ribosomal protein L37a | −3.2 | −7.21 |
| $$M12481_M_at | M12481 Mouse cytoplasmic beta-actin mRNA | −3.2 | −7.9 |
| Msa.409.0_f_at | Melanoma cell-derived intracisternal A-particle, gag gene | −3.4 | −3.87 |
| Msa.40489.0_f_a | EST | −3.4 | −6.96 |
| X06407_f_at | 21 kd polypeptide under translational control | −3.4 | −8.7 |
| ET63529_at | TcR alpha gene for T cell antigen receptor alpha-chain | −3.5 | −2.88 |
| Msa.2906.0_f_at | EST | −3.5 | −8.53 |
| x16670_f_at | Type IIB intracisternal A-particle element encoding integrase | −3.5 | −3.53 |
| X51528_f_at | Mouse gene for tum-transplantation antigen P198 | −3.6 | −8.04 |
| Msa.34708.0_s_a | EST | −3.6 | −3.11 |
| M12481_5_at | Mouse cytoplasmic beta-actin mRNA | −3.7 | −9.45 |
| X51703_s_at | Mouse mRNA for ubiquitin | −3.8 | −10.3 |
| Msa.24150.0_s_a | EST | −3.8 | −1.03 |
| x15358_f_at | Mouse mRNA for junD proto-oncogene | −3.9 | −7.66 |
| Msa.6119.0_s_at | EST | −3.9 | −1.12 |
| Msa.1406.0 at | Mus musculus zinc finger protein A20 (murine A20) mRNA | −3.9 | −3.64 |
| x04120_f_at | EST | −4 | −4.74 |
| X14061_f_at | EST | −4 | −10.3 |
| Msa.43183.0_f_a | Mouse L1Md-9 repetitive sequence | −4.1 | −6.5 |
| ET61537_g_at | B10.BR T-cell receptor alpha-chain precursor | −4.1 | −5.24 |
| M26053_g_at | T-cell receptor germline beta-chain gene constant region | −4.1 | −12 |
| Msa.7608.0_s_at | EST | −4.2 | −2.96 |
| Msa.1312.0_f_at | EST | −4.3 | −13.6 |
| Msa.1236.0_f_at | EST | −4.5 | −14 |
| Msa.2707.0_i_at | EST | −5.1 | −8.37 |
| MURINE_B2_at | EST | −5.1 | −16.5 |
| v00714_f_at | Mouse gene for alpha-globin | −5.5 | −16.4 |
| v00718_s_at | Messenger RNA for mouse pancreatic alpha-amylase | −5.6 | −2.41 |
| x13588_s_at | Murine crp gene for C-reactive protein | −5.8 | −2.19 |
| M12481_3_at | Cytoplasmic beta-actin mRNA | −5.8 | −19.9 |
| x61433_s_at | Sodium/potassium ATPase beta subunit | −9.4 | −6.58 |
| X68670_s_at | Terminal deoxynucleotidyltransferase (TdT) | −9.8 | −5.67 |
| X61385_s_at | T-cell specific transcription factor | −9.9 | −9.03 |
| Msa.19552.0_s_a | EST | −66.4 | −44.3 |

Naïve and memory B6.2.16 CD8 cells were purified as described in the Materials and Methods. cRNA was hybridized with Affymetrix Gene Chips (Mu11KA and Mu11KB). The difference in expression level of a gene between Memory and Naïve CD8 cells was evaluated by Fold Change and Sort Score, which are listed in descending order showed in Table 2 and Table 3. Genes with a Memory/Naïve ratio ≧2.0 and a sort score ≧1.0 were considered to be significantly up-regulated in memory cells.

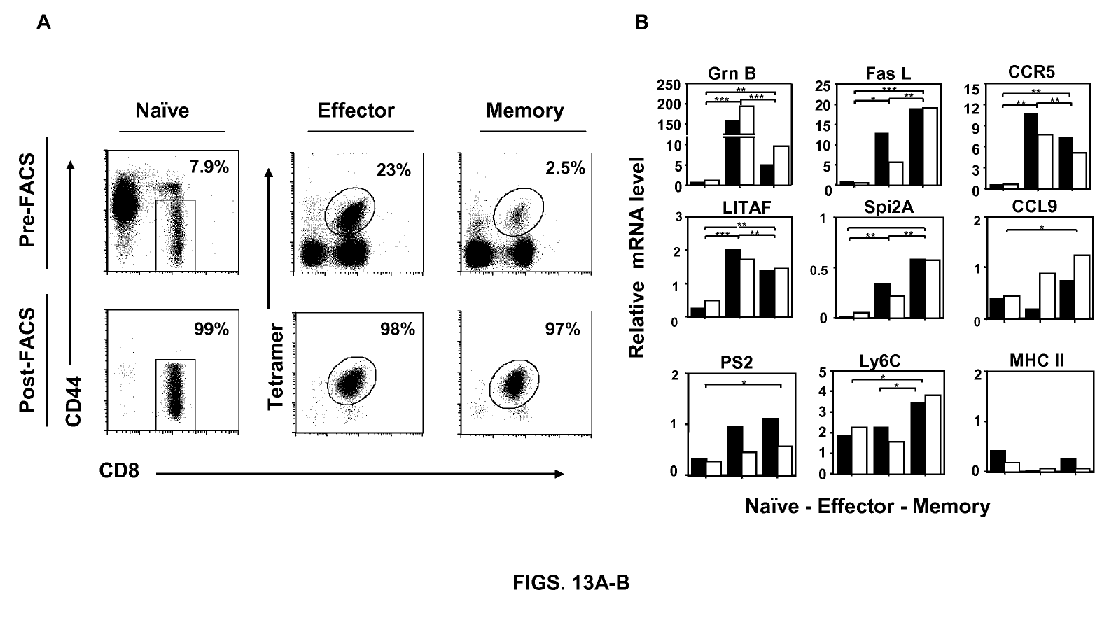

Infection of wild-type mice with LCMV Armstrong results in a vigorous CTL response, which clears the virus and gives rise to a stable pool of anti-LCMV memory CD8 cells (Lau et al., 1994; Murali-Krishna et al., 1998). To validate the physiological relevance of the candidate genes up-regulated in B6.2.16 memory CD8 cells, studies were conducted to determine whether they were also up-regulated in memory CD8 cells after infection with LCMV. Wild-type C57BL/6 mice were infected with LCMV Armstrong to generate effector CD8 cells (E) after 8 days or memory CD8 cells (M) after 80 days or more (Lau et al., 1994). Anti-LCMV CD8 cells were purified from the spleen by FACS using H-2D-tetramers loaded with three immunodominant LCMV antigen peptides and anti-CD8 antibody (FIG. 13A) (Murali-Krishna et al., 1998). Naïve CD8 cells (N) were purified directly from the spleens of un-infected C57BL/6 mice by FACS based on CD44^{low}CD8⁺ staining (FIG. 13A). Two separate isolates of RNA from effector and memory cells were purified from two independent LCMV infections, while RNA of naïve cells was from two different isolations. Real-time PCR was used to determine the relative difference in expression of mRNA for a given gene between CD8 populations (FIG. 13B) (Medhurst et al., 2000).

Eight of the forty three candidate genes that were up-regulated in B6.2.16 CD8 memory cells were also up-regulated in anti-LCMV memory cells (FIG. 13B, Table 4, and Table 5).

TABLE 4
Real-Time PCR Analysis of Differential Gene Expression in CD8 Cell
Populations.

| Accession | | Memory | Memory | Effector | |
|---|---|---|---|---|---|
| Number | Gene | Naive | Effector | Naive | Function |
| U06948 | Fas L | 22 (4) | 2 | 10 | Effector |
| | | | | | molecule |
| M64085 | Spi2A | 18 (2) | 2 | 9 | Anti-apoptosis |
| D83648 | CCR5 | 10 (4) | −2 | 15 | Cell migration |
| M12302 | Grn B | 9 (3) | −24 | 211 | Effector |
| | | | | | molecule |
| AA542220 | LITAF | 4 (2) | nsd | 5 | Transcription |
| | | | | | factor |
| U57325 | PS2 | 3 (2) | nsd | nsd | Anti-apoptosis |
| U19482 | CCL9 | 3 (5) | nsd | nsd | Cell migration |
| M18466 | Ly6C | 2 (3) | 2 | nsd | Cell adhesion |

Table 4 demonstrates the relative level of gene expression in FACS purified naïve, effectors (8 d post infection) and memory (≧80 d post infection) CD8 cells from FIG. 13A. The data is from two independent experiments from FIG. 13B. For the memory/naïve comparison, the relative level of expression from DNA array analysis of B6.2.16 CD8 cells is given in parenthesis. No statistical difference in mRNA levels between populations is indicated as nsd.

TABLE 5
Relative Expression of Candidate Genes in Memory Compared to Naive
CD8 cells

| | | Memory/Naive | |
|---|---|---|---|
| Accession Number | Gene | B6.2.16 CD8 | Anti-LCMV CD8 |
| AA165775 | ATP-binding cassette protein | 43 | b.r.d. |
| AA199380 | Similar to ATP-binding cassette protein | 32 | −1.3 |
| W29434 | Similar to interferon-inducible 1-8D protein | 28 | b.r.d. |
| AA415898 | Interferon-inducible GTPase | 27 | b.r.d. |
| AA617493 | Endoplasmic reticulum protein 29 | 24 | −1.8 |
| AA542220 | LPS-induced TNF activating factor (LITAF) | 21 | 3.0 |
| J05261 | Mouse protective protein | 16 | 1.1 |
| X62742 | H-2DM | 16 | −1.2 |
| AA711915 | Similar to jun dimerization protein | 14 | b.r.d. |
| AA474495 | Similar to NADH-cytochrome B5 reductase | 12 | −1.2 |
| AA575696 | Similar to sorting nexin 1 | 11 | 1.1 |
| U43085 | Glucocorticoid-attenuated response gene 39 | 11 | −1.5 |
| U59807 | Cystatin B | 11 | 1.1 |
| AA288280 | Cathespin C | 8.0 | 1.1 |
| AA472322 | Sorting nexin 5 | 7.9 | −1.6 |
| X56602 | Interferon-inducible 15-kDa protein | 7.9 | 1.0 |
| U72643 | Lymphocyte specific transcript | 7.2 | b.r.d. |
| AI892501 | Similar to cathepsin Z | 7.2 | 1.0 |
| AA260827 | Similar to hypothetical protein FLJ20378 | 7.0 | 1.0 |
| AA277739 | Ubiquitin specific protease 14 | 6.9 | −1.8 |
| W18601 | Similar to cytochrome c oxidase Vb subunit | 6.7 | −1.1 |
| U86137 | Telomerase protein-1 | 6.6 | −3.5 |
| X61800 | C/EBP delta | 6.6 | b.r.d. |
| U19482 | CCL9 | 5.4 | 1.7 |
| X03479 | Serum amyloid A3 | 4.9 | 1.0 |
| AA182189 | Spi-C | 4.9 | b.r.d. |
| X54966 | Cathepsin B | 4.8 | 1.0 |
| D14566 | TAP 1 | 4.8 | 1.2 |
| D83648 | CCR5 | 4.4 | 8.5 |
| U06948 | Fas L | 4.3 | 18 |
| X06746 | Krox-20 | 3.8 | −2.0 |
| U56773 | Pelle-like protein kinase | 3.4 | 1.0 |
| Z49877 | Syk | 3.3 | b.r.d. |
| X04972 | MnSOD | 3.2 | −1.3 |
| M59470 | Cystatin C | 3.1 | −1.4 |
| M18466 | Ly6C.2 | 2.9 | 1.7 |
| M12302 | Granzyme B | 2.8 | 9.4 |
| U88908 | Inhibitor of apoptosis 1 | 2.6 | −2.2 |
| X59379 | Amyloid Beta precursor | 2.2 | −2.0 |
| M64085 | Serine protease inhibitor 2A (Spi2A) | 2.2 | 22 |
| U57325 | Presenillin 2 short | 2.2 | 3.6 |
| L31532 | Bcl-2 | 2.1 | 1.3 |
| M63695 | CD1.1 | 2.1 | −1.3 |

In Table 5, naïve and memory CD8 cells expressing either anti-HY transgenic TCR B6.2.16 or endogenous TCRs specific for LCMV were purified as described in the Materials and Methods. For a given gene, the ratio of mRNA levels in memory/naïve cells is listed in descending order. For B6.2.16 CD8 cells, the expression level is from DNA array analysis and for anti-LCMV CD8 cells, the expression level is the mean ratio from real-time PCR analysis of CD8 cells from two independent experiments (FIG. 13B; Table 4). The expression of some candidate genes, which were apparently up-regulated in memory B6.2.16 CD8 cells was below the reliable detection level (b.r.d.) by real-time PCR in naïve and anti-LCMV memory CD8 cells indicating that amplification of these genes did not reach the exponential phase during the standard 40 cycle real-time PCR run. In addition, when analyzed by real-time PCR, the expression of many candidate genes, which were higher in memory B6.2.16 CD8 cells, were no higher in anti-LCMV memory cells. The discrepancies between the expression levels of these genes in B6.2.16 CD8 memory and anti-LCMV memory in this study, and with the findings of others (Grayson et al., 2001; Grayson et al., 2000; Kaech et al., 2002), may be due to a variety of factors. These may include differences in the type of antigenic stimulation, the purity of CD8 cell populations, and whether the analysis was performed on cells expressing transgenic or endogenous TCRs. Several candidate genes were up-regulated in anti-LCMV memory CD8 cells. Candidate genes that were not up-regulated in anti-LCMV memory CD8 cells (M/E mRNA ratio<1.7) were excluded from further consideration. Some candidate genes (8) had an expression level that was below the reliable detection limit by real-time PCR and so were also excluded to avoid misleading apparent, differences in expression between CD8 populations (Marrack et al., 2000).

Several genes identified by our two-stage screen have also been found by others to be up-regulated in memory CD8 cells (Grayson et al., 2001; Kaech et al., 2002). These include CTL-specific genes such as the effector molecules granzyme B and Fas L (Russell and Ley, 2002), the memory cell marker gene Ly6C (Walunas et al., 1995) and the chemokine receptor gene C—C chemokine receptor 5 (CCR5) (Bleul et al., 1997)(FIG. 13B, Table 4). To determine whether the apparent increase in expression level of candidate genes in anti-LCMV memory CD8 cells was due to contamination with non-T cells, real-time PCR was performed to detect the expression of the class II MHC gene I-A_{α}^{b} (MHC II). There was no difference in the expression of MHC II between populations of FACS-purified CD8 cells (FIG. 13B). Therefore, the two-stage assay allowed identification of genes that were up-regulated in physiologically relevant memory CD8 cells.

These analyses allowed for the identification of other genes not previously known to be up-regulated in memory CD8 cells (Table 5). Two criteria were used for selecting a candidate for a protective gene in memory CD8 cell differentiation-correlation of gene expression with memory cell development and anti-apoptotic function. Of the genes examined, FasL and Spi2A were up-regulated to the greatest extent during the development of naïve to memory cells. Importantly, the up-regulation of both these genes also correlated with the differentiation of CTLs into memory cells (Table 2). However, of the two, only Spi2A can protect against PCD, whereas FasL is a well known initiator of PCD (Kagi et al., 1994b; Liu et al., 2003). The possibility that the up-regulation of an anti-apoptotic, dominant-negative form of FasL in memory CD8 cells (Chinnaiyan et al., 1996) was detected cannot be discounted. However, this is unlikely given the previous finding that the Fas-FasL pathway of death plays no role in PCD during anti-LCMV memory CD8 cell development (Razvi et al., 1995). As with granzyme B, the expression of FasL renders memory CD8 cells capable of using effector pathways of PCD to directly kill infected cells (Russell and Ley, 2002). This suggests that the up-regulation of Spi2A in CTLs facilitates the escape of memory cell precursors from PCD.

A model to evaluate the role of Spi2A in memory CD8 cell development. To directly test whether the up-regulation of Spi2A facilitates the differentiation of memory CD8 cells, Spi2A expression in CD8 cells was modulated after infection with LCMV. Recombinant retroviruses allowed for both the elevatation and knock-down of the expression of Spi2A mRNA in CD8 cells after infection with LCMV.

Bone marrow progenitor cells from C57BL/6 mice were transduced with a retrovirus encoding both Spi2A and green fluorescent protein (GFP) on a polycistronic mRNA (Liu et al., 2003; Zang and Ren, 1998). Bone-marrow chimeras harboring empty vector (GFP mice) or retrovirus encoding Spi2A (Spi2A mice) were generated after the adoptive transfer of transduced bone-marrow into lethally irradiated C57BL/6 CD8-deficient mice (Fueng-Leung et al., 1991). It has been shown that the expression of anti-sense Spi2A mRNA abrogates the NF-κB-dependent, up-regulation of endogenous Spi2A mRNA (Liu et al., 2003). To generate CD8 cells in which Spi2A could be upregulated, bone-marrow chimeras that expressed anti-sense Spi2A mRNA encoded by retrovirus (Spi2A-A mice) were created.

The efficiency of bone-marrow transduction and subsequent engraftment was no different for any of the retroviruses (Table 6).

TABLE 6
Generation of Bone Marrow Chimeras Transduced with Retrovirus

| | Peripheral Blood | | | GFP-Positive | |
|---|---|---|---|---|---|
| Group | % CD8 | % B220 | % GFP | % CD8 | % B220 |
| GFP (n = 24) | 4.45 ± 0.26 | 47.99 ± 3.01 | 39.51 ± 4.01 | 4.46 ± 0.17 | 39.72 ± 2.49 |
| | (1.77-7.53) | (17.8-66.1) | (2.01-64.7) | (2.55-6.48) | (12.0-61.0) |
| Spi2A (n = 21) | 4.47 ± 0.19 | 45.48 ± 2.92 | 33.17 ± 3.96 | 4.76 ± 0.15 | 32.33 ± 3.6 |
| | (2.16-5.85) | (18.6-64.8) | (1.19-69.7) | (3.26-6.03) | (8.31-56.7) |
| Spi2A-A (n = 24) | 4.71 ± 0.37 | 39.39 ± 3.90 | 40.29 ± 4.24 | 4.75 ± 0.24 | 33.99 ± 3.72 |
| | (1.97-6.31) | (10.9-66.8) | (1.36-71.8) | (3.10-6.84) | (6.75-60.8) |

Wild-type C57BL/6 bone-marrow, which had been transduced with retrovirus, was adoptively transferred into lethally irradiated (1200 rads) C57BL/6 CD8-deficient mice (1.5-2.0×10⁶ cells/mouse). After 8 weeks, PBLs were analyzed for the engraftment of retrovirally-transduced (GFP-positive) B lymphocytes (B220-positive) and CD8 cells. The level of CD8 cells in C57BL/6 CD8-deficient mice was below the level of detection by FACS (<0.1% of PBLs). The mean percentage ±SEM of each lymphocyte population and the range in parenthesis are indicated. The mean level of CD8 cells in C57BL/6-deficient bone-marrow chimeras was about 50% of the level in age-matched wild-type C57BL/6 mice [8.36±0.41% (8.07−8.94%)], whereas the level of B cells was comparable [44.5±1.47% (40.4−47.7%)]. There was no significant difference in the level of CD8 cells and B cells in retrovirally-transduced PBLs (GFP-positive) between different experimental groups of chimeras.

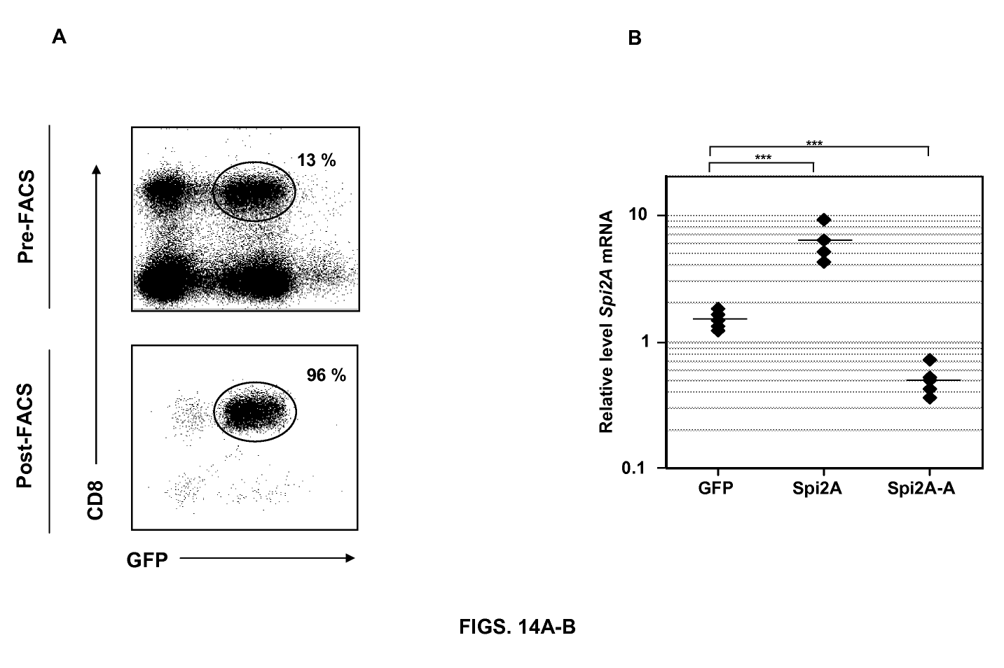

After 8 weeks, the level of CD8 cells in PBLs of C57BL/6 CD8-deficient chimeras was about 50% of the wild-type C57BL/6 control level. Of the population of transduced PBLs (GFP-positive), there was no difference in the percentage of CD8 cells between GFP, Spi2A or Spi2A-A mice (Table 6). Therefore, the expression of sense or anti-sense Spi2A in bone-marrow progenitors did not affect the development or homeostasis of naÏve CD8 cells. GFP⁺ CD8⁺ cells from the spleens of Spi2A and Spi2A-A mice were purified eight days after infection with LCMV (FIG. 14A). Real-time PCR analysis revealed a significant increase of Spi2A mRNA in CD8 cells from Spi2A mice (FIG. 14B). Importantly, the expression of anti-sense Spi2A knocked-down the expression of endogenous Spi2A mRNA in CD8 cells after infection with LCMV (FIG. 14B). These results indicate that expression of retroviral-encoded Spi2A sense and anti-sense mRNA modulates the expression of Spi2A mRNA in LCMV-specific CTLs.

Spi2A determines the level of antigen-specific CD8 cells after infection with LCMV. Programmed cell death is critical in determining the level of antigen-specific CD8 cells after infection and the resulting level of antigen-specific memory CD8 cells. Spi2A encodes an inhibitor of PCD and is up-regulated in memory CD8 cells. Therefore, Spi2A may facilitate the development of memory CD8 cells by protecting memory cell precursors from PCD after infection. Modulation of Spi2A expression in CD8 cells has a critical affect on the level of anti-LCMV CD8 cells before and during the memory phase.

Bone-marrow chimeras transduced to the same extent with each of the recombinant retroviruses (10-35% GFP positive of PBLs) and all reconstituted to 50% of wild-type C57BL/6 level of CD8 cells, were infected and the percentage of LCMV-specific CD8 cells determined by staining PBLs with H-2Db-tetramers loaded with LCMV antigen peptides.

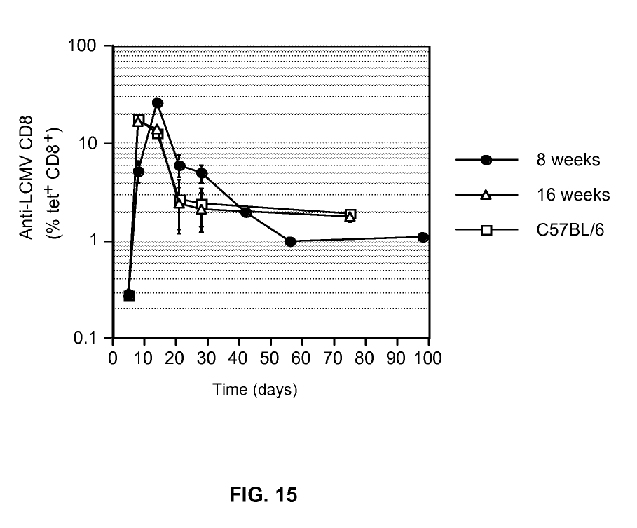

As has been previously observed, when wild-type C57BL/6 were infected with LCMV (2×10⁵ pfu/mouse), a peak number of anti-LCMV CD8 cells in PBLs was observed after 8 days (FIG. 15) (Murali-Krishna et al., 1998).

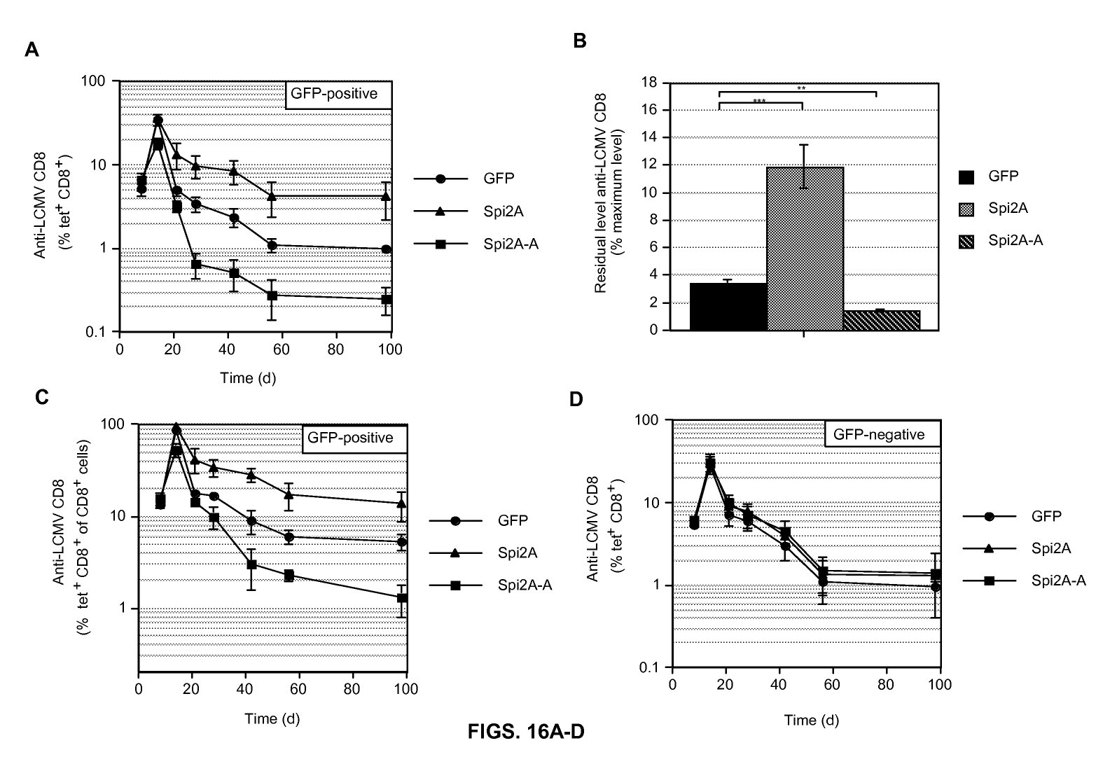

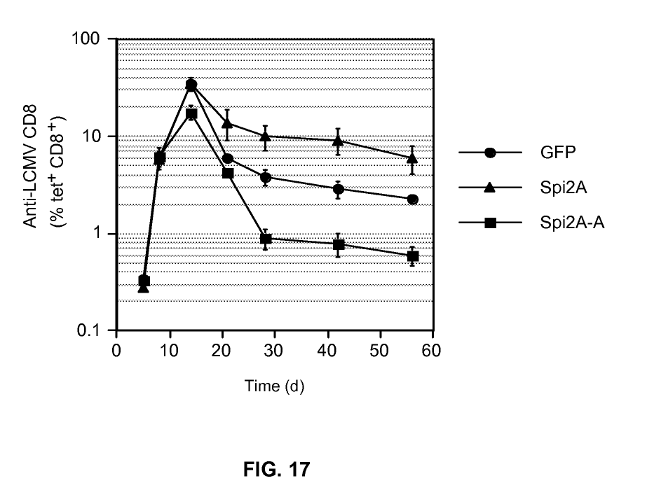

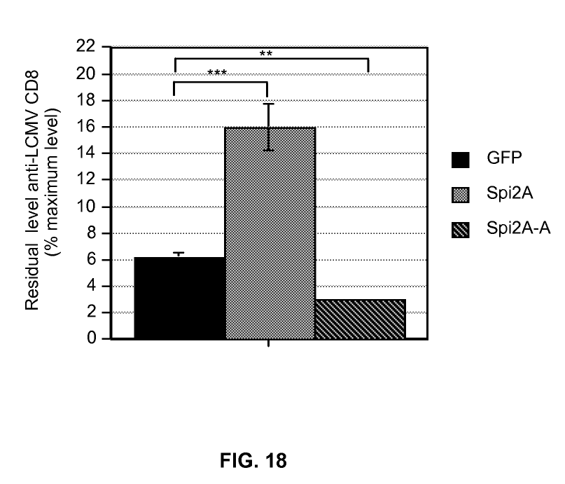

In the representative experiment in FIG. 16A, infection of GFP control chimeras, which harbored 50% of wild-type CD8 levels (FIG. 15), resulted in a rapid expansion of anti-LCMV CD8 cells, and a peak level after 14 days. The expression of recombinant Spi2A did not increase the level of anti-LCMV CD8 cells during the initial expansion phase, nor did it affect the magnitude of the peak response in Spi2A mice. However, during the ensuing contraction phase, a considerable increase in the level of anti-LCMV CD8 cells in Spi2A mice was observed, resulting in an elevated level of anti-LCMV CD8 cells during the memory phase, which was stable for at least 14 weeks (FIG. 16A; FIG. 17). In Spi2A mice, normalization of the residual level as a percentage of the maximum level revealed a significant reduction in the severity of contraction in anti-LCMV CD8 cell levels (FIG. 16B; FIG. 18). The level of anti-LCMV CD8 cells, measured as a percentage of the total GFP⁺ CD8⁺ cell population in PBLs, was also significantly elevated in Spi2A mice (FIG. 16C). Therefore, the expression of Spi2A specifically increased the levels of antigen-specific CD8 cells. In the bone-marrow chimeras, recombinant retrovirus is expressed in leucocytes other than CD8 cells (Table 6). However, no difference in the number of GFP-negative, anti-LCMV CD8 cells in Spi2A mice (FIG. 16D) was observed. Therefore, any modulation of Spi2A expression that occurred in GFP-positive leucocytes other than CD8 cells did not affect the levels of virus-specific CD8 cells. Overall, these results indicate that the expression of Spi2A in CD8 cells alleviates the severity of the contraction phase of anti-LCMV CD8 cells giving rise to an increase in the level of virus-specific cells in the memory phase.

To determine the physiological relevance of Spi2A to memory CD8 cell development, the effect of abrogating Spi2A up-regulation on the response of CD8 cells to LCMV infection was examined. Knockdown of Spi2A mRNA expression in CTLs by anti-sense message resulted in both a reduced peak level (FIG. 16A; FIG. 17) and a more severe contraction phase, as evidenced by a lower normalized residual level (FIG. 16B; FIG. 18). Thus, the level of anti-LCMV CD8 was diminished throughout the contraction and resulting memory phases (FIG. 17). It can be concluded that Spi2A determines both the peak level and the severity of the contraction phase of anti-LCMV CD8 cells. It has been long recognized that after an immune response PCD is a force limiting the number of antigen-specific CD8 cells (Ahmed and Gray, 1996). Therefore, consistent with a role as an inhibitor of PCD (Liu et al., 2003), these results indicate that the up-regulation of Spi2A is required to protect CTLs from PCD during memory CD8 cell development.

Spi2A determines the number of anti-LCMV memory CD8 cells. A characteristic of memory CD8 cells is their ability to immediately respond to re-stimulation with antigen, long after the primary exposure to antigen. To examine the role of Spi2A in the development of long-term memory cells, this phenotypic definition was used to quantitate the number of memory CD8 cells in the spleens of the recombinant retroviral bone-marrow chimeras after infection with LCMV. Modulation of Spi2A expression in CD8 cells had a significant effect on the numbers of anti-LCMV CD8 memory cells.

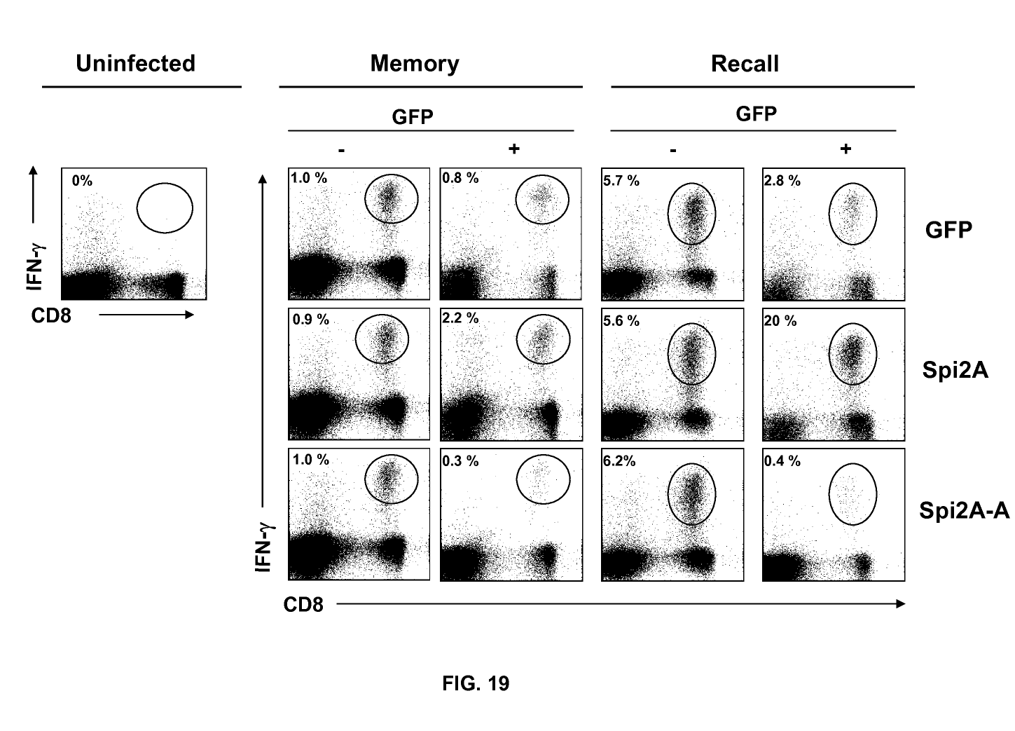

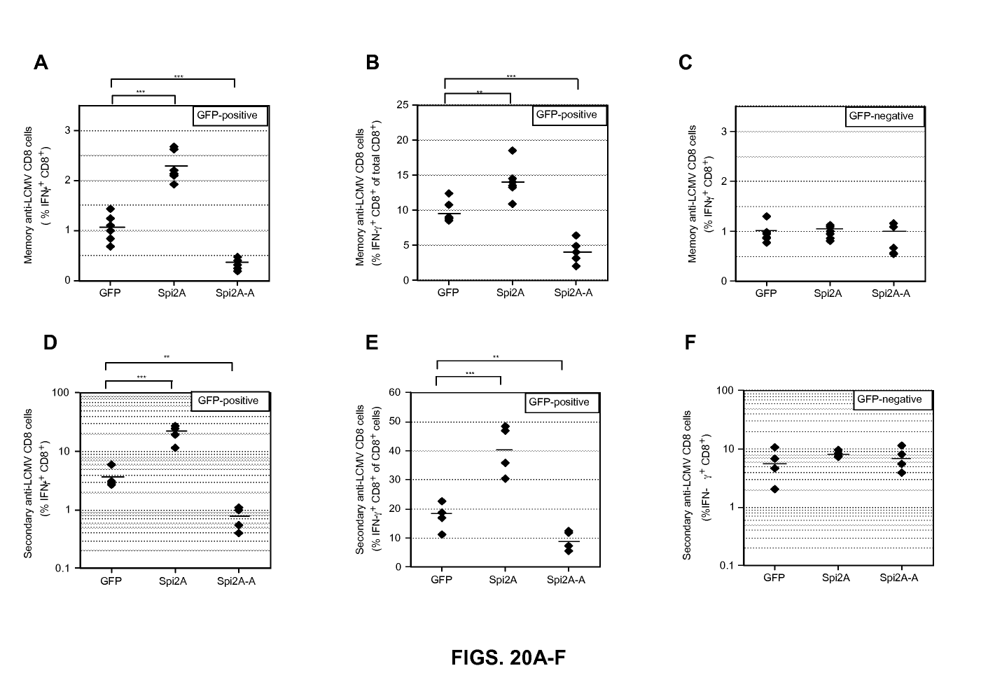

Typically, anti-LCMV-memory CD8 cells are capable of generating cytokines, such as interferon-γ (IFN-γ) within five hours of stimulation with antigen-peptides (Murali-Krishna et al., 1998). This assay was used to define and quantitate the memory CD8 cells (IFN-γ⁺ CD8⁺) that persisted in the spleen seventy-five or more days after infection with LCMV (FIG. 19). Expression of Spi2A increased the percentage (FIG. 19 and FIG. 20A) and absolute number (Table 7) of anti-LCMV CD8 cells in Spi2A mice in two independent experiments.

TABLE 7
Number of Anti-LCMV CD8 Cells in the Spleen After Infection of
Bone Marrow Chimeras

| | | Time of | | Number of anti-LCMV CD8 cells per | | |
|---|---|---|---|---|---|---|
| | | infection (d) | | spleen (×10⁻⁵) | | |
| Exp. | Chimera | Primary | Secondary | GFP⁺ | | GFP⁻ |
| 1 | GFP | 101 | n/a | 2.57 ± 0.23 | | 2.48 ± 0.38 (n = 6) |
| | Spi2A | 101 | n/a | 7.69 ± 0.94 | (199%) | 2.79 ± 0.12 (n = 6) |
| | Spi2A-A | 101 | n/a | 0.88 ± 0.12 | (−66%) | 2.56 ± 0.24 (n = 5) |
| 2 | GFP | 75 | n/a | 4.12 ± 0.55 | | 5.02 ± 0.67 (n = 3) |
| | Spi2A | 75 | n/a | 8.66 ± 1.03 | (110%) | 5.07 ± 0.68 (n = 3) |
| | Spi2A-A | 75 | n/a | 1.74 ± 0.14 | (−57%) | 5.35 ± 0.20 (n = 6) |
| 3 | GFP | 60 | 5 | 22.35 ± 4.40 | | 37.77 ± 5.67 (n = 4) |
| | Spi2A | 60 | 5 | 115.71 ± 26.13 | (417%) | 45.94 ± 7.55 (n = 4) |
| | Spi2A-A | 60 | 5 | 4.39 ± 1.28 | (−80%) | 39.26 ± 6.57 (n = 4) |

Exp. = Experiment number.

C57BL/6 CD8 deficient mice were reconstituted with bone marrow progenitors that had been transduced by retroviruses encoding GFP alone (GFP), Spi2A in the sense (Spi2A) or antisense (Spi2A-A) orientation in polycistronic messages with GFP, then infected with LCMV. Absolute cell numbers were determined for the splenocytes that were either transduced (GFP⁺) or not transduced (GFP⁻). The data in experiments 1 and 3 are further described in FIG. 20. The cell numbers are the mean values (±SEM) from the analysis of individual infected mice (n=number of mice analyzed). The numbers in parenthesis indicate the proportional change of anti-LCMV CD8 cells compared to the GFP control in each experiment. In experiments 1 and 2 mice were infected only once and so the time after the secondary infection is not applicable (n/a).

In addition, the effect of Spi2A was specific for antigen-specific CD8 cells (FIG. 20B). There was no difference in the number of GFP-negative, memory CD8 cells in Spi2A mice compared to GFP controls after infection with LCMV (FIG. 20C). Therefore, any modulation in Spi2A expression that may have occurred in non-CD8 cells in the bone-marrow chimeras was not responsible for the differences observed in memory CD8 cell recovery.

To verify that the development of memory CD8 cells facilitated by Spi2A was not due to over expression, the effect of suppressing the up-regulation of Spi2A by anti-sense message in Spi2A-A mice was examined. A significant decrease in both the percentage (FIG. 19 and FIG. 20A) and absolute number (Table 7) of anti-LCMV memory CD8 cells from Spi2A-A mice in two independent experiments was observed. Overall, it can be concluded that the up-regulation of Spi2A in CD8 cells is a physiological mechanism for facilitating the development of long-term memory CD8 cells.

Spi2A affects the potency of recall responses to LCMV. The robust secondary recall response to antigen is a characteristic of the memory phase of an immune response, and is determined by both the number and phenotype of memory T cells (Ahmed and Gray, 1996). To further examine the role of Spi2A in the development of memory CD8 cells, recall responses to LCMV in the retroviral bone-marrow chimeras were examined. As with the development of primary memory CD8 cells, Spi2A had a dramatic effect on the recall response of CD8 cells to re-infection with LCMV.

Mice were infected with LCMV to generate primary memory CD8 cells. After sixty days mice were re-challenged with LCMV and the recall response measured in the spleen five days later by determining the frequency of LCMV-reactive CD8 cells in ex vivo IFN-γ production assays (FIG. 19). As expected, re-challenge of control primary memory CD8 cells (GFP-negative) with LCMV, resulted in about a 10-fold increase in anti-LCMV CD8 cells (Blattman et al., 2000; Lin and Welsh, 1998) (Table 7). The percentages and absolute numbers of secondary anti-LCMV CD8 cells were significantly increased in Spi2A mice (FIG. 19, FIG. 20D and Table 7). In addition, the effect of Spi2A was specific for antigen-specific CD8 cells (FIG. 20E). There was no difference in the number of GFP-negative, secondary anti-LCMV CD8 cells in Spi2A mice (FIG. 20F). Therefore, any modulation in Spi2A expression in non-CD8 cells was not responsible for the effects of Spi2A on CD8 cell recall responses.

Importantly, abrogation of Spi2A up-regulation severely diminished recall responses to LCMV in Spi2A-A mice (FIG. 19, FIG. 20D and Table 7). Therefore Spi2A was a physiologically relevant determinant of CD8 cell recall responses to LCMV. Since the magnitude of recall responses is determined by both the number and phenotype of memory CD8 cells, these findings point to a critical role for Spi2A in determining CD8 cell memory to virus.

### Example 4

Assays to Identify Spi2A Polypeptide Equivalents

Using the teachings of the specification and the knowledge of those skilled in the art, one can conduct assays to identify Spi2A polypeptide equivalents. The term “Spi2A polypeptide equivalent” has been previously defined in this specification. A plurality of distinct proteins/polypeptides/peptides with different substitutions can be easily made and used in accordance with the invention. The Spi2A polypeptide equivalent can be a polypeptide from any species or organism, including a human polypeptide. In addition, the Spi2A polypeptide equivalent can be naturally occurring or synthetic polypeptide. One of ordinary skill in the art would understand that many Spi2A polypeptide equivalents would likely exist in the art and can be identified using commonly available experimental techniques. For instance, screening of libraries of known amino acid sequences can be analyzed for comparison to SEQ ID NO:2. Experimental techniques known to those of ordinary skill in the art can be used to synthesize polypeptides wherein certain, not most or all, of the amino acids of Spi2A have been substituted. One method will be to express, in E. coli, recombinant genes encoding a fusion polypeptide between a peptide of 6 histadines (His peptide) and Spi2A polypeptide or equivalent polypeptide using the pET™ (Novagen) expression cassette. These fusion polypeptides will be purified on nickel columns and the His peptide removed by protease cleavage. This method can be used to generate TAT-Spi2A polypeptides or equivalent polypeptides.

### Example 5

### Testing of Spi2A Polypeptide Equivalents

Using the teachings of the specification and the knowledge of those skilled in the art, one can conduct tests to determine whether Spi2A polypeptide equivalents can protect against both the caspase-dependent and caspase-independent pathways of cell death. For instance, it can be determined whether Spi2A polypeptide equivalents can inhibit cathepsin B activity after treatment of RelA^{+/+} MEFs with TNF-α. In addition Spi2A polypeptide equivalents can be screened for the ability to inhibit cysteine proteases in in vitro assays. For example, the Spi2A polypeptide equivalents that one can evaluate for cathepsin B inhibition may include polypeptides based on the amino acid sequence of the human serpins discussed above (i.e., SEQ ID NO:3, SEQ ID NO:4, SEQ ID NO:5, SEQ ID NO:6, SEQ ID NO:7, SEQ ID NO:8, and SEQ ID NO:9).

### Example 6

### In vivo Prevention of Tumor Development Using Spi2A Polypeptides and Spi2A Polypeptide Equivalents

Using the teachings of the specification and the knowledge of those skilled in the art, in vivo studies can be conducted to determine the ability of Spi2A polypeptides and Spi2A polypeptide equivalents to inhibit cancer in murine models of human cancer. In an initial round of in vivo trials, a mouse model of human cancer with histologic features and metastatic potential resembling those of tumors seen in humans (Katsumata et al., 1995) can be used. The animals may be treated with Spi2A polypeptides and/or Spi2A polypeptide equivalents of the present invention to determine the suppression of tumor development.

For example, Spi2A polypeptides and Spi2A polypeptide equivalents can be tested in vivo for antitumor activity against murine leukemia cell lines L1210, P388, or any other murine model of cancer known to those of skill in the art. In conjunction with these studies, the acute and sub-acute toxicities in mice may typically be studied (LD10, LD50, LD90). In a more advanced phase of testing, the antitumor activity of Spi2A polypeptides and Spi2A polypeptide equivalents against human xenografts can be assessed and cardiotoxicity studies can be done in a rat or rabbit model.

In brief, two groups of mice of a suitable cancer model can be treated with doses of Spi2A polypeptides and/or Spi2A polypeptide equivalents. Several combinations and concentrations of Spi2A polypeptides or Spi2A polypeptide equivalents can be tested. Control mice should be treated with buffer only.

The effect of Spi2A polypeptides and/or Spi2A polypeptide equivalents on the development of cancer in treated mice versus a control group can then be compared by examination of tumor size and histopathologic examination of hematoxylin and eosin stained tumor tissue.

### Example 7

### Treatment of Myocardial Infarction in Human Subjects Using Spi2A Polypeptides and Spi2A Polypeptide Equivalents

Using the teachings of the specification and the knowledge of those skilled in the art, one can design protocols that can be used to facilitate the treatment of acute myocardial infarction in human subjects using Spi2A polypeptides or Spi2A polypeptide equivalents. For example, a patient presenting with signs and symptoms clinically consistent with an acute myocardial infarction may be treated using the following protocol.

A composition of the present invention can be typically administered orally or parenterally in dosage unit formulations containing standard, well known non-toxic physiologically acceptable carriers, adjuvants, and vehicles as desired. The term parenteral as used herein includes subcutaneous injections, intravenous, intramuscular, intra-arterial injection, or infusion techniques. The Spi2A polypeptide or Spi2A polypeptide equivalent can be delivered to the patient alone or indeed in combination with other therapies for myocardial infarction. Where a combination therapy is contemplated, the Spi2A polypeptide or Spi2A polypeptide equivalent can be administered before, after or concurrently with the agents. Therapy can be administered before, after, or concurrently with cardiac catheterization or angioplasty.

For example, a treatment course can comprise about six doses delivered over a 1 to 6 day period. Upon election by the clinician the regimen may be continued at a more or less frequent basis. Of course, these are only exemplary times for treatment, and the skilled practitioner can readily recognize that many other time-courses are possible.

In one embodiment, administration may simply entail injection of the therapeutic composition intravenously. In another embodiment, a catheter can be inserted into the body and the heart is continuously perfused for a desired period of time.

Clinical responses can be defined by any acceptable measure known to those of skill in the art. For example, a complete response may be defined by improvement in cardiac function based on clinical studies well-known to those of ordinary skill in the art. Those of skill in the art can take the information disclosed in this specification and optimize the treatment regimes.

### Example 8

### Treatment of Septic Shock in Human Subjects Using Spi2A Polypeptides and Spi2A Polypeptide Equivalents

Using the teachings of the specification and the knowledge of those skilled in the art, one can design protocols to facilitate the treatment of septic shock in human subjects using Spi2A polypeptides or Spi2A polypeptide equivalents. For example, a patient presenting with signs and symptoms clinically consistent with septic shock may be treated using the following protocol.

A composition of the present invention can be administered orally or parenterally in dosage unit formulations containing standard, well known non-toxic physiologically acceptable carriers, adjuvants, and vehicles as desired. The term parenteral as used herein may include subcutaneous injections, intravenous, intramuscular, intra-arterial injection, or infusion techniques. The Spi2A polypeptide or Spi2A polypeptide equivalent can be delivered to the patient alone or indeed in combination other therapies for septic shock, such as parenteral antibiotics. Where a combination therapy is contemplated, the Spi2A polypeptide or Spi2A polypeptide equivalent can be administered before, after or concurrently with the other agents.

For example, a treatment course may comprise about six doses delivered over a 7 to 21 day period. Upon election by the clinician the regimen may be continued at a more or less frequent basis. Of course, these are only exemplary times for treatment, and the skilled practitioner can readily recognize that many other time-courses are possible.

Administration entails injection of the therapeutic composition intravenously or by other methods known to those of skill in the art. The criteria for selecting patients and protocol of administration would be as described the severe sepsis drug Xigris (Sollet and Garber, 2002; Laterre and Heiselman, 2002)

Clinical responses may be defined by improvement in clinical outcome well-known to those of ordinary skill in the art. These may include the restoration of normal blood pressure and a decrease in patient morbidity. Those of skill in the art can take the information disclosed in this specification and optimize the treatment regimes.

### Example 9

### Treatment of Cancer in Human Subjects Using Spi2A Polypeptides and Spi2A Polypeptide Equivalents

This example describes an example of a protocol to facilitate the treatment of human cancer patients using Spi2A polypeptides or Spi2A polypeptide equivalents. Patients may, but need not, have received previous chemo- radio- or gene therapeutic treatments. Optimally the patient may exhibit adequate bone marrow function (defined as peripheral absolute granulocyte count of >2,000/mm3 and platelet count of 100,000/mm3, adequate liver function (bilirubin 1.5 mg/dl) and adequate renal function (creatinine 1.5 mg/dl).

The compositions can include one or more Spi2A polypeptides or Spi2A polypeptide equivalents that may be administered parenterally in dosage unit formulations containing standard, well known non-toxic physiologically acceptable carriers, adjuvants, and vehicles as desired. The term parenteral as used herein can include subcutaneous injections, intravenous, intramuscular, intra-arterial injection, or infusion techniques. The composition may be administered directly into the tumor vasculature may be delivered to the patient alone or indeed in combination with other therapies. Where a combination therapy is contemplated, the composition may be administered before, after or concurrently with the other anti-cancer agents.

In one example, a treatment course can comprise about six doses delivered over a 7 to 21 day period. Upon election by the clinician the regimen may be continued six doses every three weeks or on a less frequent (monthly, bimonthly, quarterly etc.) basis. Of course, these are only exemplary times for treatment, and the skilled practitioner can readily recognize that many other time-courses are possible.

In one embodiment, administration may entail injection of the therapeutic composition into the tumor. In another embodiment, a catheter can be inserted into the site of the tumor and the cavity may be continuously perfused for a desired period of time.

Clinical responses can be defined by acceptable measures known to those of skill in the art. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites. Those of skill in the art can take the information disclosed in this specification and optimize the treatment regimen.

### Example 10

### Methods of Preparation of Donor Granulocytes for Delivery to a Subject in Need of a Granulocyte Donation

As discussed in the specification above, some embodiments of the present invention may pertain to use of Spi2A polypeptides and Spi2A polypeptide equivalents in the preparation of donor granulocytes. For example, one can prepare donor granulocytes by obtaining donor granulocytes from a suitable donor by means commonly known to those of skill in the art. The granulocytes can then be isolated using methods of granulocyte isolation well-known to those of skill in the art. Following isolation, the granulocytes can then be treated with a composition that includes one or more TAT-Spi2A polypeptides or TAT-Spi2A polypeptide equivalents.

In vitro studies can be conducted to compare survival of treated granulocytes to untreated controls. Blood can be collected from healthy donors which or may not have been treated with G-CSF to boost granulocyte numbers. Granulocytes can be purified by leukapheresis and TAT-Spi2A polypeptides or TAT-Spi2A polypeptide equivalents added during storage to alleviate apoptosis and neutrophil function (Hubel et al., 2001). Additional studies can be conducted in human subjects. The subject in need can be a subject with any disease or condition known to be treated with donor granulocytes. Examples of such diseases and conditions include neutropenia (due to chemotherapy, radiotherapy, myelosuppressive drugs leukemia, idiopathic neutropenia or aplastic anemia (Hubel et al., 2001), neonatal sepsis, and diseases associated with a qualitative abnormality of neutrophils such as chronic granulomatous disease. In particular the invention can be of particular usefulness in the treatment of neutropenia due to dose-intensive chemotherapy, which is amenable to transfusion therapy but not other therapies (Liles et al., 1995). Clinical trials may be performed as described in Hubel et al., 2001. Typically, neutropenic patients may receive 10-15 transfusions with 4−17×10⁹ granulocytes/m², which may have been preserved with Spi2A or equivalents. The efficacy of the agent will be measured by determining the number and ex vivo function of transferred neutrophils in patients as well as the reduction in infection with bacteria or fungi.

### Example 11

### Clinical Trials of the Use of Spi2A Polypeptides and Spi2A Polypeptide Equivalents in the Treatment of Diseases in General

This example is generally concerned with the development of human treatment protocols using the Spi2A polypeptides and Spi2A polypeptide equivalents in the treatment of diseases such as those previously discussed in this specification. In particular, such drug treatment can be of use in the clinical treatment of various diseases in which cell death and lysosomal instability play a role. Examples of these diseases include myocardial infarction, and septic shock. A more detailed example pertaining to cancer is discussed in the next example.

The various elements of conducting a clinical trial, including patient treatment and monitoring, will be known to those of skill in the art in light of the present disclosure.

The following information can be used as a general guideline for use in establishing Spi2A polypeptides and Spi2A polypeptide equivalents in clinical trials.

Patients with the targeted disease can be newly diagnosed patients or patients with existing disease. Patients with existing disease may include those who have failed to respond to at least one course of conventional therapy.

The Spi2A polypeptide or Spi2A polypeptide equivalent may be administered alone or in combination with the another therapeutic agent. The agents may be administered intravenously, orally, topically, or by another mechanism that is specific to the disease that is being treated. If intravascular, the agent may be administered during the course of intravascular procedures such as cardiac catheterization or coronary angioplasty. The agent may also be administered intraoperatively. For example, the agent may be administered directly to the heart or coronary vasculature during the course of coronary artery bypass grafting.

The starting dose may, for example, be 0.5 mg/kg body weight. Three patients may be treated at each dose level in the absence of a defined level of toxicity. Dose escalation may be done by 100% increments (e.g., 0.5 mg, 1 mg, 2 mg, 4 mg) until drug related toxicity of a specific level develops. Thereafter dose escalation may proceed by 25% increments. The administered dose may be fractionated.

The Spi2A polypeptide or Spi2A polypeptide equivalent may be administered over a short infusion time or at a steady rate of infusion over a period of days. The Spi2A infusion may be administered alone or in combination with other agents. The infusion given at any dose level will be dependent upon the toxicity achieved after each.

Physical examination, laboratory tests, and other clinical studies specific to the disease being treated may, of course, be performed before treatment and at intervals of about 3-4 weeks later. Laboratory studies can include CBC, differential and platelet count, urinalysis, SMA-12-100 (liver and renal function tests), coagulation profile, and any other appropriate chemistry studies to determine the extent of disease, or determine the cause of existing symptoms. If necessary, appropriate biological markers in serum can be monitored.

### Example 12

### Clinical Trials of the Use of Spi2A Polypeptides and Spi2A Polypeptide Equivalents in the Treatment of Cancer

This example is concerned with the development of human treatment protocols using the Spi2A polypeptides and Spi2A polypeptide equivalents in the treatment of cancer. The various elements of conducting a clinical trial, including patient treatment and monitoring, will be known to those of skill in the art in light of the present disclosure. The following information can be used as a general guideline for use in establishing Spi2A polypeptides and Spi2A polypeptide equivalents in clinical trials pertaining to cancer treatment.

Patients with cancer chosen for clinical study will typically have failed to respond to at least one course of conventional therapy. Measurable disease is not required.

The Spi2A polypeptide or Spi2A polypeptide equivalent may be administered alone or in combination with the another chemotherapeutic agent. The administration may be intravenously, directly into the tumor, topically, or in any other manner known to those of skill in the art. The starting dose may be 0.5 mg/kg body weight. Three patients may be treated at each dose level in the absence of grade >3 toxicity. Dose escalation may be done by 100% increments (0.5 mg, 1 mg, 2 mg, 4 mg) until toxicity is detected. Thereafter dose escalation may proceed by 25% increments.

The Spi2A polypeptide or Spi2A polypeptide equivalent and/or anti-cancer agent combination may be administered over a short infusion time or at a steady rate of infusion over a 7 to 21 day period. The Spi2A infusion may be administered alone or in combination with the anti-cancer drug. The infusion given at any dose level will be dependent upon the toxicity achieved after each. Increasing doses of Spi2A in combination with an anti-cancer drug will be administered to groups of patients until approximately 60% of patients show unacceptable toxicity. Doses that are ⅔ of this value could be defined as the safe dose.

Physical examination, tumor measurements, and laboratory tests can, of course, be performed before treatment and at intervals of about 3-4 weeks later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum can be monitored.

To monitor disease course and evaluate the anti-tumor responses, it is contemplated that the patients may be examined for appropriate tumor markers every 4 weeks, if initially abnormal. Laboratory studies such as a CBC, differential and platelet count, coagulation profile, and/dr SMA-12-100 shall be performed weekly. Appropriate clinical studies such as radiological studies should be performed and repeated every 8 weeks to evaluate tumor response.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

### Example 13

### Clinical Trials of the Use of Spi2A Polypeptides and Spi2A Polypeptide Equivalents in Treating Alzheimer Disease

This example is concerned with the development of human treatment protocols for the treatment and prevention of Alzheimer disease using the Spi2A polypeptides or Spi2A polypeptide equivalents developed in the present invention. For example, the Spi2A polypeptides or Spi2A polypeptide equivalents in this invention can be used to prevent amyloidosis, alone or in combination with other treatments for plaque related diseases.

The various elements of conducting a clinical trial, including patient treatment and monitoring, will be known to those of skill in the art in light of the present disclosure. The following information can be used as a general guideline for use in the treatment of amyloidosis, alone or in combination with other drugs in clinical trials.

Patients with an amyloidogenic disease or at risk of contracting such a disease can be chosen for clinical study and may have failed to respond to at least one course of conventional therapy. Measurable disease is not required. The only criterion is that these patients have or are suspected to have amyloidogenic plaques and are or have undergone fibrillogenesis.

In an exemplary clinical protocol, patients may undergo placement of a catheter, or other suitable delivery device, in a cavity will provide an effective means of delivering a therapeutic compounds of the present invention and for sampling the individual for the presence of plaque-forming amyloidogenic peptides. In the same procedure, the Spi2A polypeptides or Spi2A polypeptide equivalents may be administered alone or in combination with other therapeutic drugs that are commonly used in the treatment of Alzheimer's Disease and other amyloidogenic diseases. The administration may be regional, directly into the fibrillogenic plaque, or in a systemic manner.

The starting dose may be 0.5 mg/kg body weight. Three patients may be treated at each dose level in the absence of grade ≧3 toxicity. Dose escalation may be done by 100% increments (0.5 mg, 1 mg, 2 mg, 4 mg) until drug related grade 2 toxicity is detected. Thereafter dose escalation may proceed by 25% increments. The administered dose may be fractionated equally into two infusions, separated by six hour intervals if combined with a second drug for any given patient.

The Spi2A polypeptides or Spi2A polypeptide equivalents, and any other anti-amyloidogenic drug used in combination, may be administered over a short infusion time or at a steady rate of infusion over a 7 to 21 day period. The Spi2A polypeptides or Spi2A polypeptide equivalents may be administered by infusion, alone or in combination with the other anti-amyloidogenic drug. The infusion given at any dose level will be dependent upon the toxicity achieved after each administration. Hence, if Grade II toxicity was reached after any single infusion, or at a particular period of time for a steady rate infusion, further doses should be withheld or the steady rate infusion stopped unless toxicity improved. Increasing doses of the Spi2A polypeptides or Spi2A polypeptide equivalents alone or in combination with another anti-amyloidogenic drug will be administered to groups of patients until approximately 60% of patients show unacceptable Grade III or IV toxicity in any category. Doses that are ⅔ of this value could be defined as the safe dose.

Physical examination, plaque measurements, and laboratory tests may, of course, be performed before treatment and at intervals of about 3-4 wk later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum can be monitored.

To monitor disease course and evaluate the anti-plaque responses, it is contemplated that the patients can, for example, be examined for appropriate plaques and markers of disease every 4 wk, if initially abnormal. When measurable disease is present, plaque size measurements are to be recorded every 4 wk. Appropriate CAT scanning studies should be repeated every 8 wk to evaluate plaque response. A urinalysis may be performed every 4 wk.

Clinical responses may be defined by any acceptable measure known to those of skill in the art. For example, a complete response may be defined by the disappearance of all measurable disease for at least a month. Whereas a partial response may be defined by a 50% or greater reduction of the sum of the products of perpendicular diameters of all evaluable fibrillogenic plaques or at least 1 month with no plaque sites showing enlargement. Similarly, a mixed response may be defined by a reduction of the product of perpendicular diameters of all measurable lesions by 50% or greater with progression in one or more sites.

### Example 14

### Clinical Trials of the Use of Spi2A Polypeptides and Spi2A Polypeptide Equivalents in Treating Liver disease

Hepatic failure and cirrohsis can be treated by the administration of TAT-Spi2A polypeptides or TAT-Spi2A polypeptide equivalents by intravenous injection. It is anticipated that treatment may reduce hepatocyte necrosis and apoptosis and prevent hepatic failure and cirrohsis (Crawford, 1999). It is anticipated that trials can be under taken to treat acute liver failure and cirrohsis caused by fulminant viral hepatitis (with hepatitis A, B, C, D, E and G virus), drugs, chemicals and alcohol. In addition, TAT-Spi2A polypeptides and TAT-Spi2A polypeptide equivalents can be used to treat chronic liver disease and cirrohsis caused by viral hepatitis (with hepatitis A, B, C, D, E and G virus), drugs, chemicals and alcohol. The effect of the agent can be measured by the lowering of serum levels of hepatocyte proteins such as transaminases and a reduction in patient jaundice.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 4,797,368
U.S. Pat. No. 5,139,941
Ahmed and Gray, Science, 272:54-60, 1996.
Aksentijevich et al., Hum. Gene Ther., 7(9):1111-1122, 1996.
Al-khunaizi et al., Biochemistry, 41:3189-3199, 2002.
Baci et al., Ann. Rheum. Dis., 47:684-691, 1988.
Baci et al., Ann. Rheum. Dis., 54:281-288, 1995.
Baldwin, J. Clin. Inves., 107:241-246, 2001.
Barrett and Kirschke, Methods in Enzymol., 80:535-559, 1981.
Bash et al., Mol. Cell. Biol., 17(11):6526-6536, 1997.
Beg and Baltimore, Science, 274:782-787, 1996.
Bever and Garver, J. Neurol. Sci., 131:71-73, 1995.
Blattman et al., J. Immunol., 165:6081-6090, 2000.
Bleul et al., Proc. Natl. Acad. Sci. USA, 94:1925-1930, 1997.
Bochud and Calandra, BMJ., 326: 262-266, 2003.
Bomer and Monney, Cell Death Diff., 6:497-507, 1999.
Boussif et al., Proc. Natl. Acad. Sci. USA, 92(16):7297-7301, 1995.
Brach et al., Blood, 80: 2920-2924, 1992.
Brömme et al., Biochemistry, 38:2377-2385, 1999.
Budihardjo et al., Annu. Rev. Cell Dev. Biol., 15:269-290, 1999.
Burns et al., Proc. Natl. Acd. Sci. USA, 90:8033-8037, 1993.
Caley et al, J. Virology, 71(4):3031-3038, 1997.
Carbonelli et al., FEMS Microbiol. Lett., 177(1):75-82, 1999.
Cataldo and Nixon, Proc. Natl. Acad. Sci. USA 4, 87; 3861-3865, 1990.
Chen and Okayama, Mol. Cell. Biol., 7(8):2745-2752, 1987.
Chinnaiyan et al., J. Biol. Chem., 271(9):4961-4965, 1996.
Cocea, Biotechniques, 23(5):814-816, 1997.
Coffin, In: Virology, Fields et al., eds., Raven Press, NY, 1437-1500, 1990.
Coligan et al., In: Current Protocols in Immunology, John Wiley and Sons, New York, 1995.
Cooley et al., Biochemistry, 40:15762-15770, 2001.
Couch et al., Am. Rev. Resp. Dis., 88:394-403, 1963.
Crawford, In: Pathologic basis of disease, Cotran et al. (Eds.), 6^{th} Edition, W. B. Saunders Company, 845-901, 1999.
Davis et al., Curr. Biol., 6:146-148, 1996.
De Smaele et al., Nature, 414:308-313, 2001.
Derossi et al., J. Biol. Chem., 269:10444-10450, 1994.
Elliott and O'Hare, Cell, 88:223-233, 1997.
Fechheimer et al., Proc. Natl. Acad. Sci. USA, 84:8463-8467, 1987.
Felgner et al., Proc. Natl. Acad. Sci. USA, 84(21):7413-7417, 1987.
Ferri and Kroemer, Nat. Cell Biol., 3:E255-E263, 2001.
Foghsgaard et al., J. Cell Biol., 153:999-1009, 2001.
Foghsgaard et al., J. Cell. Biol., 153:999-1009.2001.
Fraley et al., Proc. Natl. Acad. Sci. USA, 76:3348-3352, 1979.
Franzoso et al., EMBO J., 12(10):3893-3901, 1993.
Franzoso et al., EMBO J., 15(13):3403-3412, 1996.
Franzoso et al., Nature, 359(6393):339-342, 1992.
Fueng-Leung et al., Cell, 65L443-449, 1991.
Gabizon et al., Cancer Res, 50(19):6371-8, 1996.
Ghosh and Bachhawat, In: Liver Diseases, Targeted Diagnosis and Therapy Using Specific Receptors and Ligands. Wu et al., eds., Marcel Dekker, NY, 87-104, 1991.
Glorioso et al., Mol. Biotechnol, 4(1):87-99, 1995.
Goossens et al., Proc. Natl. Acad. Sci. USA, 92:8115-8119, 1995.
Gopal, Mol. Cell. Biol., 5:1188-1190, 1985.
Graham and Van Der Eb, Virology, 52:456-467, 1973.
Grayson et al., J. Immunol., 164:3950-3954, 2000.
Grayson et al., J. Immunol., 166:795-799, 2001.
Grunhaus et al., Seminar in Virology, 200(2):535-546, 1992.
Guicciardi et al., J. Clin. Inves., 106:1127-1137, 2000.
Guicciardi et al., J. Clin. Inves., 106:1127-1137, 2000.
Hampson et al., Blood, 89:108-118, 1997.
Hampson et al., Hematol. J, 150-160, 2001.
Hanna et al., Protein Expr. Purif., 4:398-404, 1993.
Harland and Weintraub, J. Cell Biol., 101:1094-1099, 1985.
Hasel and Sutcliffe, Nucl. Acid. Res., 18:4019, 1990.
Hubel et al., J. Inf. Dis., 183: 321-328, 2001.
Hubel et al., J. Inf. Dis., 183: 321-328, 2001.
Inglis and Hill, EMBO J., 10(2):255-261, 1991.
Inglis et al., Gene, 106:213-220, 1991.
Itoh et al., Am. J. Path., 146: 1325, 1995.
Jaattela and Tschopp, Nat. Immunol., 4:416-423, 2003.
Kaech et al., Cell, 111:837-851.
Kagedal et al., Biochem. J., 359:335-343, 2001.
Kagi et al., Science, 265:528-530, 1994b.
Kajstura et al., Lab. Invest. 74: 86, 1996.
Karin and Lin, Nat. Immunol., 3:221-227, 2002.
Katsumata et al., Nat. Med., 1(7):644-648, 1995.
Kerr, et al., B. J. Cancer, 26: 239-244, 1972.
Khwaja and Tatton, J. Biol. Chem., 274:3617-36823, 1999.
Kisielow et al., Nature, 333:742-746, 1988.
Kloner et al., Circulation, 97: 1848-1853, 1998.
Kominami et al., Am. J. Pathol., 127:461-466, 1987.
Kotin et al., Proc. Natl. Acad. Sci. USA, 87(6):2211-2215, 1990.
Kyte and Doolittle, J. Mol. Biol., 157(1):105-132, 1982.
Laterre and Heiselman, Am. J. Surg., 184(6A):S39-46, 2002.
Laterre and Heiselman, Am. J. Surg., 184: 39S-46S, 2002.
Lau et al., Nature, 369:648-652, 1994.
Laughlin et al., J. Virol., 60(2):515-524, 1986.
Leavy et al., Transfusion, 40: 414-419, 2000.
Lebkowski et al., Mol. Cell. Biol., 8(10):3988-3996, 1988.
Levenson et al., Hum Gene Ther., 20; 9(8):1233-1236, 1998.
Liles et al., J. Intensive Care Med., 10: 283-293, 1995.
Lin and Welsh, J. Exp. Med., 188:1993-2001, 1998.
Linnevers et al., Protein: Sci., 6:919-921, 1997.
Liu et al., EMBO J., 22:5313-5322, 2003.
Markiewicz et al., Proc. Natl. Acad. Sci. USA, 95:3065-3070, 1998.
Marrack, Immunity, 10:735-744, 1999.
McCarty et al., J. Virol., 65(6):2936-2945, 1991.
McLaughlin et al., J. Virol, 62(6):1963-1973, 1988.
Medhurst et al., J. Neurosci. Meth., 98:9-20, 2000.
Mombaerts et al., Cell, 68:869-877, 1992.
Morris et al., Biochem. J, 371(Pt 1):165-173, 2003.
Mort et al., Arthritis Rheum., 27(5):509-515, 1984.
Mort et al., Biochem. J, 335:491-494, 1998.
Murali-Krishna et al., Adv. Exp. Med. Biol., 452:123-142, 1998.
Murali-Krishna et al., Science, 286(5443):1377-1381, 1999.
Muzyczka, Curr. Topics Microbiol. Immunol., 158: 97-129, 1992.
Nagahara et al., Nat. Med., 4(12):1449-1452, 1998.
Nicolau and Sene, Biochim. Biophys. Acta, 721:185-190, 1982.
Nixon and Cataldo, Ann NY Acad. Sci., 679:87-109, 1993.
Ober et al., Int. Immunol., 12:1353-1363, 2000.
Opferman et al., Science, 283:1745-1748, 1999.
Pelletier and Sonenberg, Nature, 334:320-325, 1988.
Poole et al., Nature, 273:545-547, 1980.
Potter et al., Proc. Natl. Acad. Sci. USA, 81:7161-7165, 1984.
Razvi et al., Am. J. Path., 147:79-91, 1995.
Rippe et al., Mol. Cell. Biol., 10:689-695, 1990.
Roux et al., Proc. Natl. Acad. Sci. USA, 86:9079-9083, 1989.
Russell and Ley, Annu. Rev. Immunol., 20:323-370, 2002.
Sambrook et al., In: Molecular cloning, Cold Spring Harbor Laboratory Press, Cold Spring Harbor, N.Y., 2001.
Samulski et al., EMBO J., 10:3941-3950, 1991.
Samulski et al., J. Virol, 63:3822-3828, 1989.
Schoen, In: Pathologic basis of disease, Cotran et al. (eds), 6^{th} Edition, W. B. Saunders Company, 543-601, 1999.
Shelling and Smith, Gene Therapy, 1:165-169, 1994.
Silverman et al., J. Biol. Chem., 276:33293-33296, 2001.
Sloane et al., Science, 212:1151-1153, 1981.
Sollet and Garber, Am. J. Surg., 184: 11S-18S, 2002.
Sollet and Graber, Am. J. Surg., 184(6A):S11-8, 2002.
Solodin et al., Biochemistry, 34(41):13537-13544, 1995.
Stegh et al., Mol. Cell. Biol., 20:5665-5679, 2000.
Stegh et al., Mol. Cell. Biol., 20:5665-5679, 2000.
Steller, Science, 267:1445-1449, 1995.
Takeda et al., Biochem. J., 288:643-648, 1992.
Thierry et al., Proc Natl Acad Sci USA. 92(21):9742-9746, 1995.
Top et al., J. Infect. Dis., 124:155-160, 1971.
Tratschin et al., Mol. Cell. Biol., 4:2072-2081, 1984.
Tsukamoto et al., Nat. Genet., 9(3):243-248, 1995.
Turk et al., Adv. Enzyme Regul., 42:285-303, 2002.
Tur-Kaspa et al., Mol. Cell. Biol., 6:716-718, 1986.
Vercammen et al., J. Exp. Med., 187:1477-1485, 1998.
Wagner et al., Science, 260:1510-1513, 1993.
Walunas et al., J. Immunol., 155:1873-1883, 1995.
Wang et al., Science, 281:1680-1683, 1998.
Wemeburg et al., Am. J. Physiol., 293:G947-G956, 2002.
White and Van der Werf, Circulation, 97: 632-638, 1998.
Wu and Wu, Biochemistry, 27:887-892, 1988.
Wu and Wu, J. Biol. Chem., 262:4429-4432, 1987.
Wyllie et al., Int. Rev. Cytol., 68:251-305, 1981.
Yang and Huang, Gene Therapy, 4 (9):950-960, 1997.
Yang et al., Proc Natl. Acad. Sci. USA, 87:9568-9572, 1990.
Zang and Ren, Blood, 92:3829-3840, 1998.
Zhang and Ren, Blood, 92:3829-3840, 1998.
Zhao et al., Genes Dev., 14:981-993, 2000.
Zhao et al., Genes Dev., 14:981-993, 2000.
Zhu et al., Science, 261(5118):209-211, 1993.

1-204. (canceled)

205. A method of promoting the development of an immune response against a target cell in a subject, comprising administering to said subject a Spi2A polypeptide or an Spi2A polypeptide equivalent.

206. The method of claim 205, wherein the target cell is a tumor cell or a cell that is infected by a pathogen.

207. The method of claim 206, wherein the target cell is a tumor cell.

208. The method of claim 207, wherein the tumor cell is a cell from a breast cancer, lung cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, or leukemia.

209. The method of claim 206, wherein the target cell is a cell that is infected by a pathogen.

210. The method of claim 209, wherein the pathogen is a virus.

211. The method of claim 210, wherein the virus is HIV, HSV, or ADV.

212. The method of claim 205, further defined as a method of inhibiting a cathepsin in a subject.

213. The method of claim 212, wherein the cathepsin is cathepsin B, cathepsin H, cathepsin L, cathepsin S, cathepsin C, cathepsin K, cathepsin O, cathepsin F, cathepsin V, cathepsin X, or cathepsin W.

214. The method of claim 205, wherein the subject has an immune disorder.

215. The method of claim 214, wherein the immune disorder is an autoimmune disorder or a disorder associated with abnormal antigen presentation.

216. The method of claim 205, wherein the cell is contacted with an Spi2A polypeptide.

217. The method of claim 205, wherein the cell is contacted with an Spi2A polypeptide equivalent.

218. The method of claim 217, wherein the Spi2A polypeptide equivalent is a polypeptide from Serpin B1, Serpin B2, Serpin B3, Serpin B4, Serpin B6, Serpin B8, or Serpin B9.

219. The method of claim 205, wherein the Spi2A polypeptide or Spi2A polypeptide equivalent is a polypeptide comprising 4 to 8 consecutive amino acid residues of the amino acid sequences MAGVGCCA or FVVAECCM.

220. The method of claim 205, wherein said Spi2A polypeptide or said Spi2A polypeptide equivalent further comprises an expression cassette comprising a promoter active in said cell, operably linked to a polynucleotide encoding an Spi2A polypeptide or an Spi2A polypeptide equivalent.

221. The method of claim 220, wherein said expression cassette is carried in a viral vector.

222. The method of claim 221, wherein said viral vector is an adenoviral vector, a retroviral vector, an adeno-associated viral vector, a vaccinia viral vector, or a pox viral vector.

223. The method of claim 220, wherein said expression cassette is carried in a nonviral vector.

224. The method of claim 223, wherein said nonviral vector is a liposome.

225. The method of claim 220, wherein the promoter is a constitutive promoter, an inducible promoter or a tissue-specific promoter.

226. The method of claim 205, wherein said Spi2A polypeptide or said Spi2A polypeptide equivalent is obtained from media of cultured cells and applied to the surface of said cell.

227. The method of claim 205, wherein said Spi2A polypeptide or said Spi2A polypeptide equivalent induces a humoral or cell-mediated immune response in the subject.

228. The method of claim 227, wherein the humoral or cell-mediated immune response is to a tumor in the subject.

229. The method of claim 227, wherein the humoral or cell-mediated immune response is to a virus.

230. The method of claim 205, further comprising administering an antigen to the subject.

231. A method for facilitating the differentiation of a T lymphocyte into a memory T lymphocyte, comprising contacting said T lymphocyte with a Spi2A polypeptide or a Spi2A polypeptide equivalent.

232. The method of claim 231, wherein the cell is contacted with an Spi2A polypeptide.

233. The method of claim 231, wherein the cell is contacted with an Spi2A polypeptide equivalent.

234. The method of claim 233, wherein the Spi2A polypeptide equivalent is a polypeptide from Serpin B1, Serpin B2, Serpin B3, Serpin B4, Serpin B6, Serpin B8, or Serpin B9.

235. The method of claim 231, wherein the Spi2A polypeptide or Spi2A polypeptide equivalent is a polypeptide comprising 4 to 8 consecutive amino acid residues of the amino acid sequences MAGVGCCA or FVVAECCM.

236. A method of inhibiting a cysteine cathepsin in a subject, comprising administering to said subject a Spi2A polypeptide or an Spi2A polypeptide equivalent.

237. The method of claim 236, wherein the cell is contacted with an Spi2A polypeptide.

238. The method of claim 236, wherein the cell is contacted with an Spi2A polypeptide equivalent.

239. The method of claim 238, wherein the Spi2A polypeptide equivalent is a polypeptide from Serpin B1, Serpin B2, Serpin B3, Serpin B4, Serpin B6, Serpin B8, or Serpin B9.

240. The method of claim 236, wherein the Spi2A polypeptide or Spi2A polypeptide equivalent is a polypeptide comprising 4 to 8 consecutive amino acid residues of the amino acid sequences MAGVGCCA or FVVAECCM.

241. A pharmaceutical composition, comprising:
(a) a Spi2A polypeptide or an Spi2A polypeptide equivalent; and
(b) an antigen, a nucleic acid encoding an antigen, or a cell expressing or presenting an antigen;
wherein the composition induces a humoral or cell-mediated immune response when administered to a subject.

242. The pharmaceutical composition of claim 241, wherein the antigen is comprised in an antigen-presenting cell.

243. The pharmaceutical composition of claim 241, further comprising an adjuvant.

244. The pharmaceutical composition of claim 241, wherein the humoral or cell-mediated immune response is against a tumor in a subject.

245. The pharmaceutical composition of claim 244, wherein the tumor is further defined as a breast cancer, lung cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, or leukemia.

246. The pharmaceutical composition of claim 241, wherein the humoral or cell-medicated immune response is against a virus in the subject.

247. The pharmaceutical composition of claim 246, wherein the virus is HIV, HSV, or ADV.
